(19) United States
(12) Patent Application Publication
(10) Pub. No.: US 2023/0302116 A1
(43) Pub. Date: Sep. 28, 2023

(54) VARIANT SARS-CoV-2 PROTEINS AND USES THEREOF

(71) Applicant: Ginkgo Bioworks, Inc., Boston, MA (US)

(72) Inventors: Ariel H. HECHT, Boston, MA (US); Seth C. Ritter, Revere, MA (US); Emily E. Wrenbeck, Milton, MA (US)

(21) Appl. No.: 17/923,327

(22) PCT Filed: May 5, 2021
(86) PCT No.: PCT/US21/30875

§ 371 Date: Nov. 4, 2022

Related U.S. Application Data
(60) Provisional application No. 63/057,654, filed on Jul. 28, 2020.
(60) Provisional application No. 63/020,978, filed on May 6, 2020.

(51) Int. Cl. A61K 39/215; C07K 14/005; A61P 31/14; C12N 15/86; G01N 33/569

(52) U.S. Cl. CPC A61K 39/215; C07K 14/005; A61P 31/14; C12N 15/86; G01N 33/56983; C12N 2770/20022; C12N 2770/20034; C12N 2770/20071; G01N 2469/20; G01N 2333/165

(57) ABSTRACT

The present disclosure relates to variants of SARS-CoV-2 nucleocapsid protein, spike protein, and spike protein receptor binding domain (RBD), as well as nucleic acids encoding such variants and methods of use thereof. Such variants are useful in diagnostic test kits and vaccines for SARS-CoV-2.

### RELATED APPLICATIONS

This application claims priority to U.S. Provisional Application 63/020,978, filed May 6, 2020, and U.S. Provisional Application 63/057,654, filed Jul. 28, 2020, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 5, 2021, is named GBB-00125_SL.txt and is 34,065,359 bytes in size.

### BACKGROUND

Coronaviruses are characterized by club-like spike proteins that project from their surface, a large RNA genome, and a unique replication strategy. Outbreaks of highly pathogenic Severe Acute Respiratory Syndrome coronavirus (SARS-CoV), Middle East Respiratory Syndrome coronavirus (MERS-CoV), and most recently SARS-CoV-2 have occurred.

COVID-19, caused by SARS-CoV-2, emerged in Wuhan, China in late 2019 and quickly spread worldwide, resulting in millions of confirmed cases and hundreds of thousands of deaths. While lockdowns, social distancing and the use of masks, gloves and personal protective equipment have slowed the spread of the disease, the successful emergence of a region or population from lockdown is dependent on the wide availability of vaccines or, at a minimum, accurate and specific diagnostic test kits for SARS-CoV-2 infection. However, vaccines are still at an early stage in development, and many of the currently available diagnostic test kits for SARS-CoV-2 lack accuracy and specificity. In addition, there are currently no vaccines to SARS-CoV-2 which have been proven to be efficacious and which are widely available.

Hence, there is an urgent need for vaccines against and accurate and specific diagnostic test kits for SARS-CoV-2.

### SUMMARY

The present disclosure relates to variants of the nucleocapsid protein, spike protein, and spike protein receptor binding domain (RBD) of SARS-CoV-2, the causative agent of COVID-19. In certain embodiments, the protein variants provided herein are optimized for immunogenicity, stability and/or other desirable qualities. In some embodiments, the variants provided herein are useful in vaccine formulations. In some embodiments, the variants provided herein are useful for preparation of diagnostic test kits for determining the presence of a SARS-CoV-2 antibody in human subjects.

In certain aspects, provided herein are SARS-CoV-2 nucleocapsid protein variants (e.g., non-naturally occurring SARS-CoV-2 nucleocapsid protein variants). In some embodiments, the amino acid sequence of the variant comprises an amino acid sequence with at least 80% at least 85% at least 90%, at least 91%, at least 92%, at least 93%, at least 94%, at least 95%, at least 96%, at least 97%, at least 98%, or at least 99% identity to amino acids 254-671 of any of SEQ ID NOs: 598 to 1194. In some embodiments, the variant comprises an amino acid sequence of amino acids 254-671 of any of SEQ ID NOs: 598 to 1194.

In certain aspects, provided herein are SARS-CoV-2 nucleocapsid protein variants (e.g., non-naturally occurring SARS-CoV-2 nucleocapsid protein variants). In some embodiments, the amino acid sequence of the variant comprises an amino acid sequence with at least 80% at least 85%, at least 90%, at least 91%, at least 92%, at least 93%, at least 94%, at least 95%, at least 96%, at least 97%, at least 98%, or at least 99% identity to amino acids 379-796 of any of SEQ ID NOs: 3202-3799. In some embodiments, the variant comprises an amino acid sequence of amino acids 379-796 of any of SEQ ID NOs: 3202-3799.

In some aspects, provided herein are SARS-CoV-2 spike protein variants (e.g., non-naturally occurring SARS-CoV-2 spike protein variants). In some embodiments, the amino acid sequence of the variant comprises an amino acid sequence with at least 80%, at least 85%, at least 90%, at least 91%, at least 92%, at least 93%, at least 94%, at least 95%, at least 96%, at least 97%, at least 98%, or at least 99% identity to amino acids 33-1230 of any of SEQ ID NOs: 1795 to 2394. In some embodiments, the variant comprises an amino acid sequence of amino acids 33-1230 of any of SEQ ID NOs: 1795 to 2394.

In some aspects, provided herein are SARS-CoV-2 spike protein variants (e.g., non-naturally occurring SARS-CoV-2 spike protein variants). In some embodiments, the amino acid sequence of the variant comprises an amino acid sequence with at least 80%, at least 85%, at least 90%, at least 91%, at least 92%, at least 93%, at least 94%, at least 95%, at least 96%, at least 97%, at least 98%, or at least 99% identity to amino acids 33-705 of any of SEQ ID NOs: 42994791. In some embodiments, the variant comprises an amino acid sequence of amino acids 33-705 of any of SEQ ID NOs: 42994791.

In some aspects, provided herein are SARS-CoV-2 spike protein receptor binding domain (RBD) variants (e.g., non-naturally occurring SARS-CoV-2 spike protein receptor binding domain (RBD) variants). In some embodiments, the amino acid sequence of the variant comprises an amino acid sequence with at least 80%, at least 85%, at least 90%, at least 91%, at least 92%, at least 93%, at least 94%, at least 95%, at least 96%, at least 97%, at least 98%, or at least 99% identity to amino acids 33-236 of any of SEQ ID NOs: 2494 to 2592. In some embodiments, the variant comprises an amino acid sequence of amino acids 33-236 of any of SEQ ID NOs: 2494 to 2592.

In some embodiments, the SARS-CoV-2 nucleocapsid protein variant (e.g., non-naturally occurring SARS-CoV-2 nucleocapsid protein variant) comprises an amino acid sequence comprises any of SEQ ID NOs: 598 to 1194 without the N-terminal MHHHHHH sequence (SEQ ID NO: 2597). In some embodiments, the SARS-CoV-2 nucleocapsid protein variant (e.g., non-naturally occurring SARS-CoV-2 nucleocapsid protein variant) comprises an amino acid sequence comprises any of SEQ ID NOs: 3202 to 3799 either with or without the N-terminal M amino acid or the C-terminal HHHHHH sequence (SEQ ID NO: 2599). In some embodiments, the SARS-CoV-2 spike protein variant (e.g., non-naturally occurring SARS-CoV-2 spike protein variant) comprises an amino acid sequence of any of SEQ ID NOs: 1795 to 2394 without the N-terminal M amino acid and/or the C-terminal HHHHHHHH sequence (SEQ ID NO: 2598). In some embodiments, the SARS-CoV-2 spike protein variant (e.g., non-naturally occurring SARS-CoV-2 spike protein variant) comprises an amino acid sequence of any of SEQ ID NOs: 42994791 without the N-terminal M amino acid and/or the C-terminal HHHHHHHHHH sequence (SEQ ID NO: 3802). In some embodiments, the SARS-CoV-2 spike protein receptor binding domain (RBD) variant (e.g., non-naturally occurring SARS-CoV-2 spike protein receptor binding domain (RBD) variant) comprises the amino acid sequence of any of SEQ ID NOs: 2494 to 2592 without the N-terminal M amino acid and/or the C-terminal HHHHHHHH sequence (SEQ ID NO: 2598).

In some aspects, provided herein are SARS-CoV-2 nucleocapsid protein variants (e.g., non-naturally occurring SARS-CoV-2 nucleocapsid protein variants), wherein the amino acid sequence of the variant is amino acids 254-671 of any of SEQ ID NOs: 598 to 1194 or amino acids 379-796 of any of SEQ ID NOs: 3203-3799. In other aspects, provided herein are SARS-CoV-2 spike protein variants (e.g., non-naturally occurring SARS-CoV-2 spike protein variants), wherein the amino acid sequence of the variant is amino acids 33-1230 of any of SEQ ID NOs: 1795 to 2394 or amino acids 33-705 of any of SEQ ID NOs: 4299-4791. Also provided herein are SARS-CoV-2 spike protein receptor binding domain (RBD) variants (e.g., non-naturally occurring SARS-CoV-2 spike protein receptor binding domain (RBD) variants), wherein the amino acid sequence of the variant is amino acids 33-236 of any of SEQ ID NOs: 2494 to 2592.

In some embodiments, a SARS-CoV-2 nucleocapsid protein variant provided herein (e.g., a non-naturally occurring SARS-CoV-2 nucleocapsid protein variant) may have the amino acid sequence of any of SEQ ID NOs: 598 to 1194, either with or without the N-terminal MHHHHHH sequence (SEQ ID NO: 2597).

In some embodiments, a SARS-CoV-2 nucleocapsid protein variant provided herein (e.g., a non-naturally occurring SARS-CoV-2 nucleocapsid protein variant) may have the amino acid sequence of any of SEQ ID NOs: 3203-3799, either with or without the N-terminal M amino acid and/or the C-terminal HHHHHH sequence (SEQ ID NO: 2599).

In some embodiments, a SARS-CoV-2 spike protein variant provided herein may have the amino acid sequence of any of SEQ ID NOs: 1795 to 2394, either with or without the N-terminal M amino acid and/or the C-terminal HHHHHHHH sequence (SEQ ID NO: 2598).

In some embodiments, a SARS-CoV-2 spike protein variant provided herein may have the amino acid sequence of any of SEQ ID NOs: 4299 to 4791, either with or without the N-terminal M amino acid and/or the C-terminal HHHHHHHHH sequence (SEQ ID NO: 3802).

In certain embodiments, a SARS-CoV-2 spike protein receptor binding domain (RBD) variant disclosed herein may have the amino acid sequence of any of SEQ ID NOs: 2494 to 2592, either with or without the N-terminal M amino acid and/or the C-terminal HHHHHHHH sequence (SEQ ID NO: 2598).

In some embodiments, a SARS-CoV-2 nucleocapsid protein variant disclosed herein may have an amino acid sequence comprising a span of at least 15 contiguous amino acids of amino acids 254-671 of any of SEQ ID NOs: 598 to 1194, wherein the span comprises at least one variation from the corresponding span of a wild-type SARS-CoV-2 nucleocapsid protein.

In some embodiments, a SARS-CoV-2 nucleocapsid protein variant disclosed herein may have an amino acid sequence comprising a span of at least 15 contiguous amino acids of amino acids 379-796 of any of SEQ ID NOs: 3202 to 3799, wherein the span comprises at least one variation from the corresponding span of a wild-type SARS-CoV-2 nucleocapsid protein.

In certain embodiments, a SARS-CoV-2 spike protein variant disclosed herein may have an amino acid sequence comprising a span of at least 15 contiguous amino acids of amino acids 33-1230 of any of SEQ ID NOs: 1795 to 2394, wherein the span comprises at least one variation from the corresponding span of a wild-type SARS-CoV-2 spike protein.

In certain embodiments, a SARS-CoV-2 spike protein variant disclosed herein may have an amino acid sequence comprising a span of at least 15 contiguous amino acids of amino acids 33-705 of any of SEQ ID NOs: 4299 to 4791, wherein the span comprises at least one variation from the corresponding span of a wild-type SARS-CoV-2 spike protein.

In some embodiments, a SARS-CoV-2 spike protein receptor binding domain (RBD) variant may have an amino acid sequence comprising amino acids 33-236 of any of SEQ ID NOs: 2494 to 2592, wherein the span comprises at least one variation from the corresponding span of a wild-type SARS-CoV-2 spike protein RBD domain.

In some embodiments, a SARS-CoV-2 nucleocapsid protein variant disclosed herein may be any variant that comprises an amino acid sequence that has a substitution, insertion or deletion (e.g., a variation from the wild-type SARS-CoV-2 nucleocapsid protein) at one or more of: amino acids at positions 50, 53, 54, 55, 56, 57, 59, 62, 65, 68, 72, 74, 76, 79, 81, 86, 87, 90.91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and/or a deletion of amino acid position 1, relative to the wild-type SARS-CoV-2 nucleocapsid protein amino acid sequence (SEQ ID NO: 2594).

In certain embodiments, a SARS-CoV-2 nucleocapsid protein variant disclosed herein may be any variant wherein the amino acid sequence of the variant has a substitution (e.g., a variation from the wild-type SARS-CoV-2 nucleocapsid protein) at one or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and/or a deletion of amino acid position 1, relative to the wild-type SARS-CoV-2 nucleocapsid protein amino acid sequence (SEQ ID NO: 2594).

In some embodiments, a SARS-CoV-2 nucleocapsid protein variant disclosed herein may be any variant wherein the amino acid sequence of the variant has a substitution, insertion or deletion (e.g., a variation from the wild-type SARS-CoV-2 nucleocapsid protein) at two or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and/or a deletion of amino acid position 1, relative to the wild-type SARS-CoV-2 nucleocapsid protein amino acid sequence (SEQ ID NO: 2594).

In certain embodiments, a SARS-CoV-2 nucleocapsid protein variant disclosed herein may be any variant wherein the amino acid sequence of the variant has a substitution (e.g., a variation from the wild-type SARS-CoV-2 nucleocapsid protein) at two or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and/or a deletion of amino acid position 1, relative to the wild-type SARS-CoV-2 nucleocapsid protein amino acid sequence (SEQ ID NO: 2594).

In some embodiments, a SARS-CoV-2 nucleocapsid protein variant disclosed herein may be any variant wherein the amino acid sequence of the variant has a substitution, insertion or deletion (e.g., a variation from the wild-type SARS-CoV-2 nucleocapsid protein) at three or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and/or a deletion of amino acid position 1, relative to the wild-type SARS-CoV-2 nucleocapsid protein amino acid sequence (SEQ ID NO: 2594).

In some embodiments, a SARS-CoV-2 nucleocapsid protein variant disclosed herein may be any variant wherein the amino acid sequence of the variant has a substitution (e.g., a variation from the wild-type SARS-CoV-2 nucleocapsid protein) at three or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and/or a deletion of amino acid position 1, relative to the wild-type SARS-CoV-2 nucleocapsid protein amino acid sequence (SEQ ID NO: 2594).

In certain embodiments, a SARS-CoV-2 spike protein variant may be any variant wherein the amino acid sequence of the variant has a substitution, insertion or deletion (e.g., a variation from the wild-type SARS-CoV-2 spike protein) at one or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and/or a deletion of amino acid positions 1 to 13, and a deletion of amino acids 1212-1273, relative to the wild-type SARS-CoV-2 spike protein amino acid sequence (SEQ ID NO: 2596).

In some embodiments, a SARS-CoV-2 spike protein variant disclosed herein may be any variant wherein the amino acid sequence of the variant has a substitution (e.g., a variation from the wild-type SARS-CoV-2 spike protein) at one or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141,142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and/or a deletion of amino acid positions 1 to 13, and a deletion of amino acids 1212-1273, relative to the wild-type SARS-CoV-2 spike protein amino acid sequence (SEQ ID NO: 2596).

In some embodiments, a SARS-CoV-2 spike protein variant disclosed herein may be any variant wherein the amino acid sequence of the variant has a substitution, insertion or deletion (e.g., a variation from the wild-type SARS-CoV-2 spike protein) at two or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and/or a deletion of amino acid positions 1 to 13, and a deletion of amino acids 1212-1273, relative to the wild-type SARS-CoV-2 spike protein amino acid sequence (SEQ ID NO: 2596).

In certain embodiments, a SARS-CoV-2 spike protein variant disclosed herein may be any variant wherein the amino acid sequence of the variant has a substitution (e.g., a variation from the wild-type SARS-CoV-2 spike protein) at two or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and/or a deletion of amino acid positions 1 to 13, and a deletion of amino acids 1212-1273, relative to the wild-type SARS-CoV-2 spike protein amino acid sequence (SEQ ID NO: 2596).

In some embodiments, a SARS-CoV-2 spike protein variant disclosed herein may be any variant wherein the amino acid sequence of the variant has a substitution, insertion or deletion (e.g., a variation from the wild-type SARS-CoV-2 spike protein) at three or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155,157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417,444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and/or a deletion of amino acid positions 1 to 13, and a deletion of amino acids 1212-1273, relative to the wild-type SARS-CoV-2 spike protein amino acid sequence (SEQ ID NO: 2596).

In certain embodiments, a SARS-CoV-2 spike protein variant disclosed herein may be any variant wherein the amino acid sequence of the variant has a substitution (e.g., a variation from the wild-type SARS-CoV-2 spike protein) at three or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and/or a deletion of amino acid positions 1 to 13, and a deletion of amino acids 1212-1273, relative to the wild-type SARS-CoV-2 spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at one or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at one or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624,625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at two or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420,424, 432, 439, 443, 445, 459, 460, 461, 464, 467, 470, 472, 475, 477, 480, 482, 484, 487, 488, 489, 490, 496, 501, 503, 504,505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at two or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at three or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at three or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, a SARS-CoV-2 spike protein receptor binding domain (RBD) variant disclosed herein may be any variant wherein the amino acid sequence of the variant has a substitution, insertion or deletion (e.g., a variation from the wild-type SARS-CoV-2 spike protein receptor binding domain (RBD) protein) at one or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acid positions 1 to 327 and a deletion of amino acid positions 532 to 1273, relative to the wild-type SARS-CoV-2 spike protein amino acid sequence (SEQ ID NO: 2596).

In certain embodiments, a SARS-CoV-2 spike protein receptor binding domain (RBD) variant disclosed herein may be any variant wherein the amino acid sequence of the variant has a substitution at (e.g., a variation from the wild-type SARS-CoV-2 spike protein receptor binding domain (RBD) protein) one or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acid positions 1 to 327 and a deletion of amino acid positions 532 to 1273, relative to the wild-type SARS-CoV-2 spike protein amino acid sequence (SEQ ID NO: 2596).

In certain embodiments, a SARS-CoV-2 spike protein receptor binding domain (RBD) variant may be any variant wherein the amino acid sequence of the variant has a substitution, insertion or deletion (e.g., a variation from the wild-type SARS-CoV-2 spike protein receptor binding domain (RBD) protein) at two or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acid positions 1 to 327 and a deletion of amino acid positions 532 to 1273, relative to the wild-type SARS-CoV-2 spike protein amino acid sequence (SEQ ID NO: 2596).

A SARS-CoV-2 spike protein receptor binding domain (RBD) variant may be any variant wherein the amino acid sequence of the variant has a substitution at two or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acid positions 1 to 327 and a deletion of amino acid positions 532 to 1273, relative to the wild-type SARS-CoV-2 spike protein amino acid sequence (SEQ ID NO: 25%).

In some embodiments, a SARS-CoV-2 spike protein receptor binding domain (RBD) variant may be any variant wherein the amino acid sequence of the variant has a substitution, insertion or deletion (e.g., a variation from the wild-type SARS-CoV-2 spike protein receptor binding domain (RBD) protein) at three or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acid positions 1 to 327 and a deletion of amino acid positions 532 to 1273, relative to the wild-type SARS-CoV-2 spike protein amino acid sequence (SEQ ID NO: 2596).

In certain embodiments, a SARS-CoV-2 spike protein receptor binding domain (RBD) variant may be any variant wherein the amino acid sequence of the variant has a substitution (e.g., a variation from the wild-type SARS-CoV-2 spike protein receptor binding domain (RBD) protein) at three or more of: amino acids at positions 335, 339, 340, 356, 357, 362, 366, 368, 372, 393, 394, 399, 402, 403, 404,405, 406, 410, 414, 417, 438, 439, 450, 477, 478, 483, 484, 486, 489, 490, 501, 502, or 504, and a deletion of amino acid positions 1 to 327 and a deletion of amino acid positions 532 to 1273, relative to the wild-type SARS-CoV-2 spike protein amino acid sequence (SEQ ID NO: 2596).

In some aspects, provided herein are compositions comprising a protein variant provided herein (e.g., a SARS-CoV-2 nucleocapsid protein variant, a SARS-CoV-2 spike protein variant, a SARS-CoV-2 spike protein RBD variant). In some embodiments, the variant is immobilized on a solid support (e.g., as a component of a diagnostic kit). In certain embodiments, the composition may be a vaccine composition. In some embodiments, the composition may further comprise a pharmaceutically acceptable carrier and/or an adjuvant.

In certain aspects, provided herein are nucleic acids encoding a variant disclosed herein and compositions comprising nucleic acids encoding a variant disclosed herein. In some embodiments, the nucleic acid may have a sequence comprising any of SEQ ID NOs: 1 to 597 (and/or a complement thereof). The nucleic acid may have a sequence comprising any of SEQ ID NOs: 1195 to 1794 (and/or a complement thereof). The nucleic acid may have a sequence comprising any of SEQ ID NOs: 2395 to 2493 (and/or a complement thereof). The nucleic acid may comprise DNA or RNA (e.g., an mRNA molecule, such as an mRNA encoding a protein variant disclosed herein).

Disclosed herein are protein variants with any mutation disclosed herein. The protein variants disclosed herein may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty of substitution mutations disclosed herein.

In certain aspects, provided herein is a composition comprising a nucleic acid disclosed herein. The composition comprising a nucleic acid encoding a variant disclosed herein may be a vaccine composition. The composition may further comprise a pharmaceutically acceptable carrier and/or an adjuvant.

In some embodiments, provided herein are vectors comprising the nucleic acids disclosed herein. Also provided herein are compositions comprising vectors comprising the nucleic acids disclosed herein. The vector may be an expression vector. In some embodiments, provided herein is a composition comprising such a vector (e.g., a vaccine composition). In certain embodiments, the composition may further comprise a pharmaceutically acceptable carrier and/or an adjuvant.

In certain aspects, provided herein are cells (e.g., host cells) comprising the nucleic acids disclosed herein and/or expressing a variant protein provided herein. The cell may express the variant encoded by the nucleic acid. The cell may be a prokaryotic cell (e.g., a bacterial cell), or a eukaryotic cell (e.g., a yeast cell, mammalian cell, or a human cell). In certain embodiments, provided are methods of generating a variant protein provided herein comprising culturing cells under conditions such that they express a variant protein disclosed herein. In certain embodiments, the methods further comprise purifying the variant protein.

In some embodiments, also provided herein are diagnostic test kits. The kits may comprise a protein variant disclosed herein. The variant may be a SARS-CoV-2 nucleocapsid protein variant, a SARS-CoV-2 spike protein variant, or a SARS-CoV-2 spike protein receptor binding domain (RBD) variant. In some embodiments, the kit further comprises a reagent capable of detecting an immunological complex which comprises an antibody bound to the variant. The diagnostic test kit may comprise at least two of the variants disclosed herein (e.g., two variants selected from a SARS-CoV-2 nucleocapsid protein variant; a SARS-CoV-2 spike protein variant; and a SARS-CoV-2 spike protein receptor binding domain (RBD) variant). The kit may comprise one or more control standards and/or a specimen diluent and/or washing buffer. The variant may be immobilized on a solid support (e.g., a microplate well). The immunological complex may be detected by ELISA or lateral flow.

Provided herein are methods of determining whether a subject is infected with and/or has been infected with SARS-CoV-2. In some embodiments, the methods may comprise contacting a sample from the subject (e.g., a serum sample from the subject) with any one of the protein variants disclosed herein (e.g., a protein variant described herein immobilized on a solid support). The method may further comprise determining whether a SARS-CoV-2 antibody present in the sample binds the protein variant disclosed herein (e.g., detecting a complex comprising the variant protein and an antibody from the sample), wherein if the protein variant disclosed herein forms an immunological complex with the antibody, the subject is infected and/or has been infected with SARS-CoV-2. The sample may be a biological sample that comprises antibodies that are capable of binding to any one of the variants provided herein (e.g., a serum sample). In some embodiments, the variant is immobilized on a solid support (e.g., a microplate well, a flow cell). In some embodiments, the immunological complex may be detected by ELISA or lateral flow.

Also provided herein are methods of treating and/or preventing a SARS-CoV-2 infection comprising administering a composition disclosed herein (e.g., a vaccine composition disclosed herein). The method may comprise administering one more (e.g. two or more, three or more, four or more, five or more, or six or more) doses of the composition.

Provided herein are methods of making a variant disclosed herein, the method comprising transfecting a cell with a nucleic acid disclosed herein. The method may further comprise expanding the cell into a population of cells, and isolating the peptides translated from the nucleic acids disclosed herein from the population of cells. The cell may be a prokaryotic cell (e.g., a bacterial cell), or a eukaryotic cell (e.g., a yeast cell, mammalian cell, or human cell).

### BRIEF DESCRIPTION OF THE FIGURES

FIG. 1 is a schematic showing a plasmid used to express ECT spike antigen proteins in Expi293F cells. The coding sequence for the ECT spike antigens (labeled “Library Gene”) was driven by the CMV promoter. The plasmid contains markers for both HEK293 (NTP II marker) and bacteria (AMP Marker), as well as an origin of replication for bacteria (pBR322).

FIG. 2 depicts a graph showing protein purification yield data of ECT spike antigens expressed from Expi293F cells and purified with Ni-NTA affinity chromatography. Antigen ID 4155326, representing the template (starting) antigen sequence, was used as a positive control and for determining hit ranking of the library members. The data show the plotting of three technical replicates of protein concentration measured via absorbance at 280 nm on a Nanodrop instrument. Antigen IDs and their corresponding purification yields from these graphs are shown in Table 8. SEQ ID NOs for each variant tested can be found in Table 8.

FIG. 3A depicts a graph showing Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for ACE2.

FIG. 3B depicts a graph showing Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for nAb1.

FIG. 3C depicts a graph showing Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for nAb2.

FIG. 3D depicts a graph showing Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for nAb3.

FIG. 4A shows repeat Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for ACE2.

FIG. 4B shows repeat Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for nAb1.

FIG. 4C shows repeat Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for nAb2.

FIG. 4D shows repeat Octet association constant (kₒₙ) binding characterization data of purified ECT spike antigen variants. SPN-C52H8 (SARS-CoV 2 Spike protein with His Tag) was purchased from Acro Biosystems and used as a positive control in the experiment. Results are for measured kₒₙ for nAb3.

FIG. 5 depicts graphs showing correlation analysis of Octet association constant (kₒₙ) binding characterization data of purified ECT spike antigen variants. R² numbers indicate the calculated Pearson correlation coefficient.

### DETAILED DESCRIPTION

In certain aspects, the present disclosure pertains to variants of a SARS-CoV-2 nucleocapsid protein, variants of a SARS-CoV-2 spike protein, and variants of a SARS-CoV-2 spike protein receptor binding domain (RBD). In some embodiments, one or more variants of one, two or all three SARS-CoV-2 proteins can be used in a diagnostic test kit for the virus. In some embodiments, one or more variants of one, two or all three SARS-CoV-2 proteins can be used in a vaccine for the treatment and/or prevention of SARS-CoV-2 infection.

To support the COVID-19 pandemic response, computational protein engineering was used to design variants with enhanced stability while retaining key immunogenic sites. Variants are expressed in bacterial and mammalian cells, purified using a validated high-throughput (HT) purification platform, and screened for sensitivity, specificity, and stability. Such a large-scale approach to antigen optimization has yet to be applied to diagnostics development, and represents a significant advancement in the field while addressing a critical gap in the public health response to COVID-19.

The variants provided herein are optimized antigens that facilitate sensitive and specific SARS-CoV-2 vaccines and point of care (PoC) serological diagnostics.

In some embodiments, the serological diagnostic assays can be used to diagnose infections as early as 3 days after onset of symptoms, as well as immunity persistent for months to years after exposure to a virus or vaccine. In certain embodiments, the serological tests are used to distinguish those who are already immune to a virus, and can resume normal activities, from those who are not and must be protected from infection.

In some embodiments, the PoC tests provided herein can be performed outside a clinical laboratory (e.g., a doctor's office, school, or home) and can yield results within minutes without needing specialized equipment. Such serological PoC tests will be critical in efforts to mitigate SARS-CoV-2 transmission, given the naivete of the global population to the virus, its apparently high contagiousness, and the fact that a substantial proportion of those infected may be asymptomatic. In some embodiments, the serological PoC tests provided herein are economical and can scale to meet global demand.

In certain embodiments, the protein variants provided herein serve as antigens in serological PoC tests. In some embodiments, such antigens can capture anti-SARS-CoV-2 antibodies present in patient samples; binding of a secondary antibody converts the primary binding event into a detectable signal (e.g., a color change) that is used to determine a positive or negative test result.

Serology tests have two fundamental performance metrics that depend indirectly on antigen choice: sensitivity, the true positive rate; and specificity, the true negative rate. Sensitivity and specificity need to be high (typically greater than 95%) for a test to be diagnostically useful. High specificity, in particular, is essential for discriminating SARS-CoV-2-seropositive individuals from those who are seroconverted due to exposure to distinct but closely related pathogens. To enable a viable PoC diagnostic, the antigen must satisfy an additional criterion, stability, for robustness to storage conditions and immobilization on the device matrix.

Conventional antigen development involves generating and testing relatively few (<5) antigenic viral proteins against clinical serum samples with respect to sensitivity and specificity. A major shortcoming of this approach is its variable success rate. Because so few antigens are typically tested, success can come in a few weeks, if the few antigens were chosen “wisely,” or can take as long as a year or more, if the “right” antigen is akin to the needle in the haystack (e.g., if the antigen is inherently unstable or exists in multiple conformations) (Avouba 2016, Lyashchenko 2000). This problem is amenable to a highly parallelized solution: testing many antigen variants from the start.

Several SARS-CoV-2 serological tests—lateral flow assays (LFA) and enzyme-linked immunosorbent assays (ELISA)—have been reported (Amanat 2020, Li 2020, Xiang 2020) or offered for sale (Healgen, Native Antigen Company, Raybiotech, Sheridan 2020). Many claim adequate sensitivity and specificity, but the number of patient-derived sera samples used to validate the assays were much smaller than recommended (Buderer 1996), and thus these assays may not hold up to more rigorous scrutiny.

Design and test of large antigen libraries will accelerate and de-risk PoC serological diagnostics development.

To accelerate and de-risk PoC diagnostic development, thousands of candidate antigenic proteins and variants derived therefrom are tested. This approach is applicable to the COVID-19 pandemic as well as to future infectious disease outbreaks.

In certain embodiments, variants are designed to have enhanced stability. Notably, the unstable immunogenic pre-fusion conformation of the SARS-CoV-2 S protein was stabilized for cryo-electron microscopy (cryo-EM) by inclusion of two proline mutations in the S2 fusion domain (Wrapp 2020). Despite these modifications, expression titers were low, suggesting room for further improvement in stability, which correlates with expression level (Wrenbeck 2019). Amanat et al. expressed a similar S mutant with furin cleavage site removed, and separately expressed only the receptor binding domain (RBD) (Amanat 2020). Although both constructs were used to create ELISA assays, it remains unclear how well these specific antigens will perform in the context of an LFA device, wherein antigens must be retained on a solid surface and be shelf-stable. Additional stabilizing mutations can be introduced to further improve stability. In certain embodiments, variants are designed to have enhanced specificity.

Sequence- and structure-based computational engineering pipeline is used to predict, and then implement, mutations that enhance thermodynamic (robustness to high temperatures) and colloidal (reduced aggregation propensity) stability and specificity, all while preserving binding functionality. A comparable technique increased the thermostability of the malaria invasion protein RH5 by 10 to 15° C. for use as an antigen in vaccine development (Compoetto 2017). The structural data available for SARS-CoV-2 S protein (Walls 2020; Wrapp 2020) suggests similar strategies can be applied here.

The HT platform enables performing up to 8,000 arrayed bacterial or yeast transformations and 1,000 mammalian transfections weekly. N antigen libraries will be transformed into E. coli. S libraries will be transfected into mammalian cells. Protein expression cultures will be prepared in 96-well format, facilitating downstream assays. 8,000 arrayed samples per week will be purified and quantified.

The library is screened in vitro for binding to human anti-SARS-CoV-2 antibodies and stability by the four approaches mentioned above. Controls will include antibodies specific for other coronaviruses, e.g., SARS-CoV-1, MERS. NL63, 229E, and HKU1 spiked into human serum.

### Definitions

For convenience, certain terms employed in the specification, examples, and appended claims are collected here.

As used herein, the term “administering” means providing a pharmaceutical agent (e.g., a variant provided herein) and/or a composition to a subject (e.g., a vaccine composition provided herein), and includes, but is not limited to, administering by a medical professional and self-administering.

The term “amino acid” is intended to embrace all molecules, whether natural or synthetic, which include both an amino functionality and an acid functionality and capable of being included in a polymer of naturally occurring amino acids. Example amino acids include naturally occurring amino acids, analogs, derivatives and congeners thereof: amino acid analogs having variant side chains; and all stereoisomers of any of any of the foregoing.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement. In some embodiments, the nucleic acid molecule is a messenger RNA (mRNA). As used herein, the term “messenger RNA” (mRNA) refers to any polynucleotide which encodes a polypeptide of interest (e.g., a protein variant disclosed herein) and which is capable of being translated to produce the encoded polypeptide of interest in vitro, in vivo, in situ or ex vivo.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of an antibody to bind to a predetermined antigen or the ability of a polypeptide to bind to its predetermined binding partner. By way of non-limiting example, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, cascin).

As used herein, the term “subject” means a human or non-human animal selected for treatment or therapy.

The phrases “therapeutically effective amount” and “effective amount” as used herein mean the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Variants of SARS-CoV-2 Nucleocapsid Protein, Variants of S ARS-CoV-2 Spike Protein, and Variants of SARS-CoV-2 Spike Protein Receptor Binding Domain (RBD)

In some embodiments, the present disclosure pertains to novel variants of SARS-CoV-2 nucleocapsid protein, variants of SARS-CoV-2 spike protein, and variants of SARS-CoV-2 spike protein RBD. In some embodiments, a variant has a high immunogenicity, high specificity, high stability, and/or other qualities which make it useful as a component in a diagnostic test kit.

In some embodiments the variants provided herein comprise a different sequence from a corresponding SARS-CoV-2 protein. Wild-type SARS-CoV-2 nucleocapsid and spike amino acid and nucleic acid sequences can be found below:

SARS-COV-2 nucleocapsid wild-type nucleic
acid sequence (SEQ ID NO: 2593):
ATGTCTGATAATGGACCCCAAAATCAGCGAAATGCACCCCGCATTACGTT
TGGTGGACCCTCAGATTCAACTGGCAGTAACCAGAATGGAGAACGCAGTG
GGGCGCGATCAAAACAACGTCGGCCCCAAGGTTTACCCAATAATACTGCG
TCTTGGTTCACCGCTCTCACTCAACATGGCAAGGAAGACCTTAAATTCCC
TCGAGGACAAGGCGTTCCAATTAACACCAATAGCAGTCCAGATGACCAAA
TTGGCTACTACCGAAGAGCTACCAGACGAATTCGTGGTGGTGACGGTAAA
ATGAAAGATCTCAGTCCAAGATGGTATTTCTACTACCTAGGAACTGGGCC
AGAAGCTGGACTTCCCTATGGTGCTAACAAAGACGGCATCATATGGGTTG
CAACTGAGGGAGCCTTGAATACACCAAAAGATCACATTGGCACCCGCAAT
CCTGCTAACAATGCTGCAATCGTGCTACAACTTCCTCAAGGAACAACATT
GCCAAAAGGCTTCTACGCAGAAGGGAGCAGAGGCGGCAGTCAAGCCTCTT
CTCGTTCCTCATCACGTAGTCGCAACAGTTCAAGAAATTCAACTCCAGGC
AGCAGTAGGGGAACTTCTCCTGCTAGAATGGCTGGCAATGGCGGTGATGC
TGCTCTTGCTTTGCTGCTGCTTGACAGATTGAACCAGCTTGAGAGCAAAA
TGTCTGGTAAAGGCCAACAACAACAAGGCCAAACTGTCACTAAGAAATCT
GCTGCTGAGGCTTCTAAGAAGCCTCGGCAAAAACGTACTGCCACTAAAGC
ATACAATGTAACACAAGCTTTTGGCAGACGTGGTCCAGAACAAACCCAAG
GAAATTTTGGGGACCAGGAACTAATCAGACAAGGAACTGATTACAAACAT
TGGCCGCAAATTGCACAATTTGCCCCCAGCGCTTCAGCGTTCTTCGGAAT
GTCGCGCATTGGCATGGAAGTCACACCTTCGGGAACGTGGTTGACCTACA
CAGGTGCCATCAAATTGGATGACAAAGATCCAAATTTCAAAGATCAAGTC
ATTTTGCTGAATAAGCATATTGACGCATACAAAACATTCCCACCAACAGA
GCCTAAAAAGGACAAAAAGAAGAAGGCTGATGAAACTCAAGCCTTACCGC
AGAGACAGAAGAAACAGCAAACTGTGACTCTTCTTCCTGCTGCAGATTTG
GATGATTTCTCCAAACAATTGCAACAATCCATGAGCAGTGCTGACTCAAC
TCAGGCCTAA
SARS-COV-2 nucleocapsid wild-type amino
acid sequence (SEQ ID NO: 2594):
TQHGKEDLKFPRGQGVPINTNSSPDDQIGYYRRATRRIRGGDGKMKDLSP
RWYFYYLGTGPEAGLPYGANKDGIIWVATEGALNTPKDHIGTRNPANNAA
IVLQLPQGTTLPKGFYAEGSRGGSQASSRSSSRSRNSSRNSTPGSSRGTS
PARMAGNGGDAALALLLLDRLNQLESKMSGKGQQQQGQTVTKKSAAEASK
KPRQKRTATKAYNVTQAFGRRGPEQTQGNFGDQELIRQGTDYKHWPQIAQ
FAPSASAFFGMSRIGMEVTPSGTWLTYTGAIKLDDKDPNFKDQVILLNKH
IDAYKTFPPTEPKKDKKKKADETQALPQRQKKQQTVTLLPAADLDDFSKQ
LQQSMSSADSTQA
SARS-COV-2 spike protein wild-type nucleic
acid sequence (SEQ ID NO: 2595):
ATGTTTGTTTTTCTTGTTTTATTGCCACTAGTCTCTAGTCAGTGTGTTAA
TCTTACAACCAGAACTCAATTACCCCCTGCATACACTAATTCTTTCACAC
GTGGTGTTTATTACCCTGACAAAGTTTTCAGATCCTCAGTTTTACATTCA
ACTCAGGACTTGTTCTTACCTTTCTTTTCCAATGTTACTTGGTTCCATGC
TATACATGTCTCTGGGACCAATGGTACTAAGAGGTTTGATAACCCTGTCC
TACCATTTAATGATGGTGTTTATTTTGCTTCCACTGAGAAGTCTAACATA
ATAAGAGGCTGGATTTTTGGTACTACTTTAGATTCGAAGACCCAGTCCCT
ACTTATTGTTAATAACGCTACTAATGTTGTTATTAAAGTCTGTGAATTTC
AATTTTGTAATGATCCATTTTTGGGTGTTTATTACCACAAAAACAACAAA
AGTTGGATGGAAAGTGAGTTCAGAGTTTATTCTAGTGCGAATAATTGCAC
TTTTGAATATGTCTCTCAGCCTTTTCTTATGGACCTTGAAGGAAAACAGG
GTAATTTCAAAAATCTTAGGGAATTTGTGTTTAAGAATATTGATGGTTAT
TTTAAAATATATTCTAAGCACACGCCTATTAATTTAGTGCGTGATCTCCC
TCAGGGTTTTTCGGCTTTAGAACCATTGGTAGATTTGCCAATAGGTATTA
ACATCACTAGGTTTCAAACTTTACGGTGCTGCAGCTTATTATGTGGGTTA
TCTTCAACCTAGGACTTTTCTATTAAAATATAATGAAAATGGAACCATTA
CAGATGCTGTAGACTGTGCACTTGACCCTCTCTCAGAAACAAAGTGTACG
TTGAAATCCTTCACTGTAGAAAAAGGAATCTATCAAACTTCTAACTTTAG
AGTCCAACCAACAGAATCTATTGTTAGATTTCCTAATATTACAAACTTGT
GCCCTTTTGGTGAAGTTTTTAACGCCACCAGATTTGCATCTGTTTATGCT
TGGAACAGGAAGAGAATCAGCAACTGTGTTGCTGATTATTCTGTCCTATA
TAATTCCGCATCATTTTCCACTTTTAAGTGTTATGGAGTGTCTCCTACTA
AATTAAATGATCTCTGCTTTACTAATGTCTATGCAGATTCATTTGTAATT
AGAGGTGATGAAGTCAGACAAATCGCTCCAGGGCAAACTGGAAAGATTGC
TGATTATAATTATAAATTACCAGATGATTTTACAGGCTGCGTTATAGCTT
GGAATTCTAACAATCTTGATTCTAAGGTTGGTGGTAATTATAATTACCTG
TATAGATTGTTTAGGAAGTCTAATCTCAAACCTTTTGAGAGAGATATTTC
AACTGAAATCTATCAGGCCGGTAGCACACCTTGTAATGGTGTTGAAGGTT
TTAATTGTTACTTTCCTTTACAATCATATGGTTTCCAACCCACTAATGGT
GTTGGTTACCAACCATACAGAGTAGTAGTACTTTCTTTTGAACTTCTACA
TGCACCAGCAACTGTTTGTGGACCTAAAAAGTCTACTAATTTGGTTAAAA
ACAAATGTGTCAATTTCAACTTCAATGGTTTAACAGGCACAGGTGTTCTT
ACTGAGTCTAACAAAAAGTTTCTGCCTTTCCAACAATTTGGCAGAGACAT
TGCTGACACTACTGATGCTGTCCGTGATCCACAGACACTTGAGATTCTTG
ACATTACACCATGTTCTTTTGGTGGTGTCAGTGTTATAACACCAGGAACA
AATACTTCTAACCAGGTTGCTGTTCTTTATCAGGATGTTAACTGCACAGA
AGTCCCTGTTGCTATTCATGCAGATCAACTTACTCCTACTTGGCGTGTTT
ATTCTACAGGTTCTAATGTTTTTCAAACACGTGCAGGCTGTTTAATAGGG
GCTGAACATGTCAACAACTCATATGAGTGTGACATACCCATTGGTGCAGG
TATATGCGCTAGTTATCAGACTCAGACTAATTCTCCTCGGCGGGCACGTA
GTGTAGCTAGTCAATCCATCATTGCCTACACTATGTCACTTGGTGCAGAA
AATTCAGTTGCTTACTCTAATAACTCTATTGCCATACCCACAAATTTTAC
TATTAGTGTTACCACAGAAATTCTACCAGTGTCTATGACCAAGACATCAG
TAGATTGTACAATGTACATTTGTGGTGATTCAACTGAATGCAGCAATCTT
TTGTTGCAATATGGCAGTTTTTGTACACAATTAAACCGTGCTTTAACTGG
AATAGCTGTTGAACAAGACAAAAACACCCAAGAAGTTTTTGCACAAGTCA
AACAAATTTACAAAACACCACCAATTAAAGATTTTGGTGGTTTTAATTTT
TCACAAATATTACCAGATCCATCAAAACCAAGCAAGAGGTCATTTATTGA
AGATCTACTTTTCAACAAAGTGACACTTGCAGATGCTGGCTTCATCAAAC
AATATGGTGATTGCCTTGGTGATATTGCTGCTAGAGACCTCATTTGTGCA
CAAAAGTTTAACGGCCTTACTGTTTTGCCACCTTTGCTCACAGATGAAAT
GATTGCTCAATACACTTCTGCACTGTTAGCGGGTACAATCACTTCTGGTT
GGACCTTTGGTGCAGGTGCTGCATTACAAATACCATTTGCTATGCAAATG
GCTTATAGGTTTAATGGTATTGGAGTTACACAGAATGTTCTCTATGAGAA
CCAAAAATTGATTGCCAACCAATTTAATAGTGCTATTGGCAAAATTCAAG
ACTCACTTTCTTCCACAGCAAGTGCACTTGGAAAACTTCAAGATGTGGTC
AACCAAAATGCACAAGCTTTAAACACGCTTGTTAAACAACTTAGCTCCAA
TTTTGGTGCAATTTCAAGTGTTTTAAATGATATCCTTTCACGTCTTGACA
AAGTTGAGGCTGAAGTGCAAATTGATAGGTTGATCACAGGCAGACTTCAA
AGTTTGCAGACATATGTGACTCAACAATTAATTAGAGCTGCAGAAATCAG
AGCTTCTGCTAATCTTGCTGCTACTAAAATGTCAGAGTGTGTACTTGGAC
AATCAAAAAGAGTTGATTTTTGTGGAAAGGGCTATCATCTTATGTCCTTC
CCTCAGTCAGCACCTCATGGTGTAGTCTTCTTGCATGTGACTTATGTCCC
TGCACAAGAAAAGAACTTCACAACTGCTCCTGCCATTTGTCATGATGGAA
AAGCACACTTTCCTCGTGAAGGTGTCTTTGTTTCAAATGGCACACACTGG
TTTGTAACACAAAGGAATTTTTATGAACCACAAATCATTACTACAGACAA
CACATTTGTGTCTGGTAACTGTGATGTTGTAATAGGAATTGTCAACAACA
CAGTTTATGATCCTTTGCAACCTGAATTAGACTCATTCAAGGAGGAGTTA
GATAAATATTTTAAGAATCATACATCACCAGATGTTGATTTAGGTGACAT
CTCTGGCATTAATGCTTCAGTTGTAAACATTCAAAAAGAAATTGACCGCC
TCAATGAGGTTGCCAAGAATTTAAATGAATCTCTCATCGATCTCCAAGAA
CTTGGAAAGTATGAGCAGTATATAAAATGGCCATGGTACATTTGGCTAGG
TTTTATAGCTGGCTTGATTGCCATAGTAATGGTGACAATTATGCTTTGCT
GTATGACCAGTTGCTGTAGTTGTCTCAAGGGCTGTTGTTCTTGTGGATCC
TGCTGCAAATTTGATGAAGACGACTCTGAGCCAGTGCTCAAAGGAGTCAA
ATTACATTACACATAA
SARS-CoV-2 spike protein wild-type nucleic
acid sequence (SEQ ID NO: 2596):
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNERVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQUITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

### Variants of SARS-CoV-2 Nucleocapsid Protein

In some aspects, various novel variants of SARS-CoV-2 nucleocapsid protein are described herein. In some embodiments, various novel variants of SARS-CoV-2 nucleocapsid protein are described herein and annotated in Table 1. In some embodiments, a variant of SARS-CoV-2 nucleocapsid protein comprises at least one substitution, deletion and/or insertion of any variant of SARS-CoV-2 nucleocapsid protein described herein.

TABLE 1
Annotation of example nucleocapsid amino acid sequences
provided in the sequence listing (SEQ ID NOs: 598-1194).

| Nucleocapsid aa ORF splits | start | end |
|---|---|---|
| Start Codon | 1 | 1 |
| Molecule/1106289 [dsDNA] (Tag \| 6xHis) (SEQ ID NO: 2599)) | 2 | 7 |
| Molecule/180976 [dsDNA] (Linker \| 4aa GGSG) (SEQ ID NO: 2600) | 8 | 11 |
| Molecule/1106083 [dsDNA] (GST - encoded for Escherichia coli) | 12 | 242 |
| Molecule/180976 [dsDNA] (Linker \| 4aa GGSG) (SEQ ID NO: 2600)) | 243 | 246 |
| Molecule/1106295 [dsDNA] (TEV - encoded for Escherichia coli) | 247 | 253 |
| Nucleocapsid variant | 254 | 671 |

In some embodiments, the present disclosure pertains to: A non-naturally occurring variant of the SARS-CoV-2 nucleocapsid protein, wherein the amino acid sequence of the variant is amino acids 254-671 of any of SEQ ID NOs: 598 to 1194.

In some embodiments, the present disclosure pertains to: A non-naturally occurring variant of the SARS-CoV-2 nucleocapsid protein, wherein the amino acid sequence of the variant comprises a span of at least 15 contiguous amino acids of amino acids 254-671 of any of SEQ ID NOs: 598 to 1194, wherein the span comprises at least one variation from the corresponding span of the wild-type SARS-CoV-2 nucleocapsid protein.

In some embodiments, the present disclosure pertains to: A non-naturally occurring variant of the SARS-CoV-2 nucleocapsid protein, wherein the amino acid sequence of the variant has a substitution, insertion or deletion at one or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of amino acid position 1, relative to the wild-type nucleocapsid protein amino acid sequence (SEQ ID NO: 2594).

In some embodiments, a deletion of amino acid position 1 is described as d1 or D1 or A1.

In some embodiments, the present disclosure pertains to: A non-naturally occurring variant of the SARS-CoV-2 nucleocapsid protein, wherein the amino acid sequence of the variant has a substitution (e.g., a variation from the wild-type SARS-CoV-2 nucleocapsid protein) at one or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of amino acid position 1, relative to the wild-type nucleocapsid protein amino acid sequence (SEQ ID NO: 2594).

In some embodiments, the present disclosure pertains to: A non-naturally occurring variant of the SARS-CoV-2 nucleocapsid protein, wherein the amino acid sequence of the variant has a substitution, insertion or deletion (e.g., a variation from the wild-type SARS-CoV-2 nucleocapsid protein) at two or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115,118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of amino acid position 1, relative to the wild-type nucleocapsid protein amino acid sequence (SEQ ID NO: 2594).

In some embodiments, the present disclosure pertains to: A non-naturally occurring variant of the SARS-CoV-2 nucleocapsid protein, wherein the amino acid sequence of the variant has a substitution (e.g., a variation from the wild-type SARS-CoV-2 nucleocapsid protein) at two or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of amino acid position 1, relative to the wild-type nucleocapsid protein amino acid sequence (SEQ ID NO: 2594).

In some embodiments, the present disclosure pertains to: A non-naturally occurring variant of the SARS-CoV-2 nucleocapsid protein, wherein the amino acid sequence of the variant has a substitution, insertion or deletion (e.g., a variation from the wild-type SARS-CoV-2 nucleocapsid protein) at three or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of amino acid position 1, relative to the wild-type nucleocapsid protein amino acid sequence (SEQ ID NO: 2594).

In some embodiments, the present disclosure pertains to: A non-naturally occurring variant of the SARS-CoV-2 nucleocapsid protein, wherein the amino acid sequence of the variant has a substitution (e.g., a variation from the wild-type SARS-CoV-2 nucleocapsid protein) at three or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114,115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of amino acid position 1, relative to the wild-type nucleocapsid protein amino acid sequence (SEQ ID NO: 2594).

In some embodiments, the present disclosure pertains to: A non-naturally occurring variant of the SARS-CoV-2 nucleocapsid protein, wherein the amino acid sequence of the variant comprises amino acids 254-671 of any of SEQ ID NOs: 598 to 1194.

In some embodiments, the present disclosure pertains to: A non-naturally occurring variant of the SARS-CoV-2 nucleocapsid protein, wherein the amino acid sequence of the variant comprises any of SEQ ID NOs: 598 to 1194.

In some embodiments, the present disclosure pertains to: A non-naturally occurring variant of the SARS-CoV-2 nucleocapsid protein, wherein the amino acid sequence of the variant is amino acids 254-671 any of SEQ ID NOs: 598 to 1194.

An isolated recombinant nucleic acid encoding a non-naturally occurring variant of the SARS-CoV-2 nucleocapsid protein, wherein the nucleic acid has a sequence comprising any of SEQ ID NOs: 1 to 597, or the complementary sequence thereof.

In some embodiments, the present disclosure pertains to: A host cell comprising an isolated recombinant nucleic acid encoding a non-naturally occurring variant of the SARS-CoV-2 nucleocapsid protein, wherein the nucleic acid has a sequence comprising any of SEQ ID NOs: 1 to 597, or the complementary sequence thereof.

### Additional Variants of SARS-CoV-2 Nucleocapsid Protein

In some aspects, various novel variants of SARS-CoV-2 nucleocapsid protein are described herein. In some embodiments, various novel variants of SARS-CoV-2 nucleocapsid protein are described herein in Table 2. In some embodiments, a variant of SARS-CoV-2 nucleocapsid protein comprises at least one substitution, deletion and/or insertion of any variant of SARS-CoV-2 nucleocapsid protein described herein.

TABLE 2
Annotation of additional example nucleocapsid amino acid sequences
provided in the sequence listing (SEQ ID NOs 3203-3799).

| Nucleocapsid aa ORF splits | start | end |
|---|---|---|
| Start Codon | 1 | 1 |
| Molecule/2048679 [dsDNA] (MBP \| MALE_ECOLI \| malE \| Escherichia coli \| | 2 | 367 |
| Maltose/maltodextrin-binding periplasmic protein \| d1-26 d393-396 \| E. coli recode 1) | | |
| Molecule/180976 [dsDNA] (Linker \| 4aa GGSG) (SEQ ID NO: 2600) | 368 | 371 |
| Molecule/1106295 [dsDNA] (TEV - encoded for Escherichia Coli. Constraints: Gen9, | 372 | 378 |
| Twist, Common Restriction Enzymes, Standard Ginkgo Encoding Constraints) | | |
| Nucleocapsid variant | 379 | 796 |
| Molecule/180976 [dsDNA] (Linker \| 4aa GGSG) (SEQ ID NO: 2600)) | 797 | 800 |
| Molecule/1106289 [dsDNA] (Tag \| 6xHis) (SEQ ID NO: 2599)) | 801 | 806 |

TABLE 3
Sequences of components of additional nucleocapsid
amino acid sequences provided in Table 2.

| Nucleocapsid | Functional | nt |
|---|---|---|
| aa ORF splits | description | |
| Start Codon | start codon | atg |
| Molecule/ | solubility | aaaatcgaagaaggtaaactggtaatctggattaacggcgataaaggctataacggtct |
| 2048679 | tag | cgctgaagtcggtaagaaattcgagaaagataccggaattaaagtcaccgttgagcatc |
| [dsDNA] (MBP\| | | cggataaactggaagagaaattcccacaggttgcggcaactggcgatggccctgacatt |
| MALE_ECOLI\| | | atcttctgggcacacgaccgctttggtggctacgctcaatctggcctgttggctgaaat |
| malE\| | | caccggcgctggataaagaactgaaagcgaaaggtaagagcgcgctgatgttcaacctg |
| Escherichia | | caagaaccgtacttcacctggccgctgattgctgctgacgggggttatgogttcaagta |
| coli\| | | tgaaaacggcaagtacgacattaaagacgtgggcgtggataacgctggcgcgaaagcgg |
| Maltose/malto | | gtctgaccttcctggttgacctgattaaaaacaaacacatgaatgcagacaccgattac |
| dextrin- | | tccatcgcagaagctgcctttaataaaggcgaaacagcgatgaccatcaacggcccgtg |
| binding | | ggcatggtccaacatcgacaccagcaaagtgaattatggtgtaacggtactgccgacct |
| periplasmic | | tcaagggtcaaccatccaaaccgttcgttggcgtgctgagogcaggtattaacgccgcc |
| protein\|d1-26 | | agtccgaacaaagagctggcgaaagagttcctcgaaaactatctgctgactgatgaagg |
| d393-396\|E. | | tctggaagcggttaataaagacaaaccgctgggtgccgtagcgctgaagtcttacgagg |
| coli recode 1) | | aagagttggcgaaagatccacgtattgccgccaccatggaaaacgcccagaaaggtgaa |
| | | atcatgccgaacatcccgcagatgtccgctttctggtatgccgtgcgtactgcggtgat |
| | | caacgccgccagcggtcgtcagactgtcgatgaagccctgaaagacgcgcagact |
| | | (SEQ ID NO: 2601) |
| Molecule/ | linker | ggtggttctggt (SEQ ID NO: 2602) |
| 180976 [dsDNA] | | |
| (Linker\|4aa | | |
| GGSG) (SEQ | | |
| ID NO: 2600)) | | |
| Molecule/ | protease cut | gagaacctgtactttcaaagc (SEQ ID NO: 2603) |
| 1106295 | site | |
| [dsDNA] (TEV - | | |
| encoded for | | |
| Escherichia | | |
| Coli | | |
| Constraints: | | |
| Gen9, Twist, | | |
| Common | | |
| Restriction | | |
| Enzymes, | | |
| Standard | | |
| Ginkgo | | |
| Encoding | | |
| Constraints) | | |
| Nucleocapsid | nucleocapsid | cagaatggtgagcgtagtggagccaggtctaagcagcgccgtccccagggtctgccgaa |
| variant | | caacacggcgagctggtttactccgcttgttcaatttggtaaagaagacttaaaattcc |
| | | cgccgggccagggcgtcccgatcaacactaactcttccccggatgatcagattggttat |
| | | taccgcagagcaactcgtcggattcgcggcggcgacggtaaaatgaaagatttgccacc |
| | | gaagtggtacttttactatctcggcaccggtccgcacgctgatctgccctacgggcaaa |
| | | agaaggacggggtagtgtgggtggctcgacctggtgccctgaatactccaaaagatcat |
| | | atcggcacccgtaaccctgctaataacgctgcgatcccactgcaactgcctccgggtac |
| | | aaaactgccaaaaggcttctatgcagaagggtcgcgtggtggcagccaggcgagtaggc |
| | | aggtaacggtggtgacgctgctctggcgctgctactgctggacaggctgaaccagctgg |
| | | agtcgaagatgagtggtaaaggccaacagcagcagggccagaccgttaccaaaaagagc |
| | | gcagctgaagcatctaaaaaaccgcgacagaacgatcaggaattaatcogccaggggac |
| | | cgactacaaacactggccgcaaattgcccagttcgccccatccgctagtgcttgatgac |
| | | aaggacccgaactttaaagaccaggttattctgcttaacaaacatattgatgcctataa |
| | | aactttcccgccgaccgaacccaaaaaggacaaaaagaaaaaagctgatgaaacccagg |
| | | ctctacogcagcgtcagaaaaaacagcaaactgtgactctgctgccagctgcggacctg |
| | | gatgatttttccaagcagctgcaacagtctatgagctctgcggactctacgcaggct |
| | | (SEQ ID NO: 2604) |
| Molecule/ | linker | ggtggttctggt (SEQ ID NO: 2602) |
| 180976 [dsDNA] | | |
| (Linker\|4aa | | |
| GGSG) (SEQ | | |
| ID NO: 2600)) | | |
| Molecule/ | purification | caccatcatcaccatcat (SEQ ID NO: 2605) |
| 1106289 | tag | |
| [dsDNA] | | |
| (Tag\|6xHis) | | |
| (SEQ ID NO: | | |
| 2599)) | | |

In some embodiments, the present disclosure pertains to: A non-naturally occurring variant of the SARS-CoV-2 nucleocapsid protein, wherein the amino acid sequence of the variant comprises amino acids 379-796 of any of SEQ ID NOs: 3202-3799.

In some embodiments, the present disclosure pertains to: A non-naturally occurring variant of the SARS-CoV-2 nucleocapsid protein, wherein the amino acid sequence of the variant comprises any of SEQ ID NOs: 3202-3799.

In some embodiments, the present disclosure pertains to: A non-naturally occurring variant of the SARS-CoV-2 nucleocapsid protein, wherein the amino acid sequence of the variant is amino acids 379-796 any of SEQ ID NOs: 3202-3799.

An isolated recombinant nucleic acid encoding a non-naturally occurring variant of the SARS-CoV-2 nucleocapsid protein, wherein the nucleic acid has a sequence comprising any of SEQ ID NOs: 2606-3202, or the complementary sequence thereof.

In some embodiments, the present disclosure pertains to: A host cell comprising an isolated recombinant nucleic acid encoding a non-naturally occurring variant of the SARS-CoV-2 nucleocapsid protein, wherein the nucleic acid has a sequence comprising any of SEQ ID NOs: 2606-3202, or the complementary sequence thereof.

### Amino Acid Substitutions of Variants of SARS-CoV-2 Nucleocapsid Protein

In some embodiments, the present disclosure pertains to: A non-naturally occurring variant of the SARS-CoV-2 nucleocapsid protein, wherein the amino acid sequence of the variant comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the following mutations A50L, A50V, A50Y, F53Y, T54Q, A55G, A55P, L56I, T57K, T57V, H59F, H59K, H59N, H59Q, E62Q, K65S, K65T, R68A, R68E, R68K, R68P, R68Q, V72A, V721, V72M, V72T, V72W, I74D, I74K, 174L, I74T, I74V, T76A, T76D, T76E, T76K, T76N, T76P, T76Q, T76S, S79K, S79N, S79Q, S79T, D81S, Y86A, Y86C, Y86D, Y86E, Y86G, Y86H, Y86K, Y86M, Y86N, Y86P, Y86Q, Y86R, Y86S, Y86T, Y86W, Y87W, A90C, A90D, A90E, A90F, A90H, A901, A90K, A90L, A90M, A90Q, A90R, A90S, A90T, A90V, A90Y, T91I, T91Q, T91V, I94F, I94K, I94M, I94V, I94W, I94Y, K100Q, M101K, M101R, S105A, S105D, S105K, S105L, S105N, S105P, R107A, R107E, R107F, R107K, R107L, R107N, R107S, R107V, R107Y, Y109A, Y109F, Y109H, Y109S, Y111N, L113H, L113I, L113S, L113T, G114R, T115H, T115S, E118H, E118Y, G120A, G120D, G120E, G120H, G120K, G120N, G120Q, G120S, G120T, Y123F, A125D, A125E, A125H, A125Q, A125S, A125T, N126A, N126D, N126E, N126I, N126K, N126Q, N126R, N126S, N126T, N126V, K127H, K127M, K127Q, D128A, D128E, D128N, D128P, D128Q, D128S, D128T, D128V, I130V, I131F, I131H, I131L, I131T, I131V, I131Y, A134H, T135A, T135E, T135G, T135H, T135I, T135K, T135M, T135Q, T135R, T135S, T135V, E136D, E136K, E136N, E136P, L139D, L139K, L139M, L139N, L139Q, L139R, N140T, T141K, D144G, D144S, D144T, T148D, T148K, T148N, T148S, T148V, N150D, A152D, A152E, A152N, N154F, N154H, N154K, N154Q, N154Y, A155D, A155E, A155P, A155Q, A155S, A155T, I157A, I157C, I157D, I157E, I157F, I157G, I157H, I157K, I157L, I157M, I157N, I157P, I157Q, I157R, I157S, I157T, I157V, I157W, I157Y, V158A, V158C, V158D, V158E, V158G, V158H, V158K, V158L, V158M, V158N, V158P, V158Q, V158R, V158S, V158T, V158W, V158Y, L159C, L159I, L159N, L159T, L159V, Q160R, L161F, P162A, Q163A, Q163D, Q163E, Q163K, Q163N, Q163P, Q163S, Q163T, T166E, T166G, T166K, T166Q, T166S, T166V, T166W, L167I, L167P, K169E, K169P, G170N, A173C, A173M, A173N, A173S, S188E, S201E, S202E, S206E, S250D, S250K, S250N, S250T, A252D, A254M, A254N, S255A, K257A, K257H, K257I, K257R, K257V, Q260C, Q260W, A264P, A267D, A267E, A267F, A267G, A267H, A267K, A267N, A267Q, A267S, A267T, A267V, Y268C, Y268F, A273C, F274Y, R276A, R276E, R276K, R276L, R276P, R276S, R276T, E280D, E280H, E280K, E280N, E280Q, E280R, Q281D, Q281K, Q281P, T282L, Q289A, Q289D, Q289E, Q289K, Q289L, Q289S, E290D, E290K, E290N, E290Q, L291F, L291M, 1292L, 1292N, 1292T, 1292V, R293E, R293K, Q294E, Q294K, Q294L, Q294N, T296S, T296V, D297E, Y298D, K299P, K299S, H300G, H300K, H300N, H300Q, H300R, H300Y, 1304L, A308I, A308L, A308V, A311T, A311V, S312A, S312Q, G316F, R319E, R319H, R319K, R319Q, G321D, G321E, G321K, G321S, G321T, M322A, M322L, M322S, M322T, M322V, E323K, E323R, E323T, E323V, V324A, V324C, V324D, V324E, V324G, V324H, V324I, V324K, V324L, V324M, V324N, V324P, V324Q, V324R, V324S, V324T, V324W, V324Y, T325A, T325N, T325Q, T325S, S327D, S327N, T329I, T329V, A336K, A336S, A336T, K338T, D341E, D341K, D341P, D341Q, D341R, D341S, K342D, K342N, K342S, K342T, D343N, K347D, K347E, K347L, K347N, K347Q, K347R, D348E, D348K, D348N, I351E, I351K, K355E, K355Q, and/or Y360W relative to the wild-type nucleocapsid protein amino acid sequence (SEQ ID NO: 2594).

### Variants of SARS-CoV-2 Spike Protein

In some embodiments, various novel variants of SARS-CoV-2 spike protein are described herein. In some embodiments, various novel variants of SARS-CoV-2 spike protein are described herein and annotated in Table 4.

TABLE 4
Annotation of example spike protein amino acid sequences
provided in the sequence listing (SEQ ID NOs: 1795-2394).

| Spike aa ORF | start | end |
|---|---|---|
| Molecule/17747 [dsDNA] (Start codon) | 1 | 1 |
| Molecule/4155324 [dsDNA] (secretion signal mu-phosphatase) | 2 | 32 |
| Spike variant | 33 | 1230 |
| Molecule/4155309 [dsDNA] (linker \| GSGR(SEQ ID NO: 3800) \| H. sapiens | 1231 | 1234 |
| recode 1) | | |
| Molecule/4155318 [dsDNA] (Epitope \| TEV protease \| H. sapiens recode 1) | 1235 | 1241 |
| Molecule/4155310 [dsDNA] (linker \| GGGSG (SEQ ID NO: 3801) \| H. sapiens | 1242 | 1246 |
| recode 1) | | |
| Molecule/4155311 [dsDNA] (T6 fibritin foldon trimerization domain \| H. | 1247 | 1273 |
| sapiens recode 1) | | |
| Molecule/4155312 [dsDNA] (Tag \| 8xHis (SEQ ID NO: 2598\| H. sapiens | 1274 | 1281 |
| recode 1) | | |

In some embodiments, a variant of SARS-CoV-2 spike protein comprises at least one substitution, deletion and/or insertion of any variant of SARS-CoV-2 spike protein described herein (e.g., SEQ ID NOs 1795-2394).

In some embodiments, the present disclosure pertains to: A non-naturally occurring variant of the SARS-CoV-2 spike protein, wherein the amino acid sequence of the variant comprises amino acids 33-1230 of any of SEQ ID NOs: 1795 to 2394.

In some embodiments, the present disclosure pertains to: A non-naturally occurring variant of the SARS-CoV-2 spike protein, wherein the amino acid sequence of the variant comprises any of SEQ ID NOs: 1795 to 2394.

In some embodiments, the present disclosure pertains to: A non-naturally occurring variant of the SARS-CoV-2 spike protein, wherein the amino acid sequence of the variant is amino acids 33-1230 of any of SEQ ID NOs: 1795 to 2394.

In some embodiments, the present disclosure pertains to: A non-naturally occurring variant of the SARS-CoV-2 spike protein, wherein the amino acid sequence of the variant is any of SEQ ID NOs: 1795 to 2394.

In some embodiments, the present disclosure pertains to: A non-naturally occurring variant of the SARS-CoV-2 spike protein, wherein the amino acid sequence of the variant comprises a span of at least 15 contiguous amino acids of amino acids 33-1230 of any of SEQ ID NOs: 1795 to 2394, wherein the span comprises at least one variation from the corresponding span of the wild-type SARS-CoV-2 spike protein.

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at one or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of amino acids positions at 1 to 13, and a deletion of amino acids at positions 1212-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at one or more of: amino acids at positions 32, 33, 41, 42, 46, 47, 50, 68, 71, 77, 78, 83, 84, 87, 90, 94, 95, 100, 101,108, 112, 113, 114, 129,132, 134, 135, 137, 138,140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of amino acids at positions 1- to 13, and a deletion of amino acids at positions 1212-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at two or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of amino acids at positions 1-13, and a deletion of amino acids 1212-1273, relative to the wild-type spike protein amino acid sequence (SEQ ID NO: 25%).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at two or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960,961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of amino acids at positions 1 to 13, and a deletion of amino acids at positions 1212-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at three or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of amino acids at positions 1-13, and a deletion of amino acids at positions 1212-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at three or more of: amino acids at positions 32, 33, 41, 42, 46, 47, 50, 68, 71, 77, 78, 83, 84, 87, 90, 94, 95, 100, 101, 108, 112, 113, 114, 129,132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of amino acids at positions 1 to 13, and a deletion of amino acids at positions 1212-1273, relative to the wild-type spike protein amino acid sequence (SEQ ID NO: 25%).

An isolated recombinant nucleic acid encoding a non-naturally occurring variant of the SARS-CoV-2 spike protein, wherein the nucleic acid has a sequence set forth in SEQ ID NOs. 1195-1794, or the complementary sequence thereof.

In some embodiments, the present disclosure pertains to: A host cell comprising an isolated recombinant nucleic acid encoding a non-naturally occurring variant of the SARS-CoV-2 spike protein, wherein the nucleic acid has a sequence set forth in SEQ ID NOs. 1195-1794, or the complementary sequence thereof.

### Additional Variants of SARS-CoV-2 Spike Protein

In some embodiments, various additional novel variants of SARS-CoV-2 spike protein are described herein. In some embodiments, various novel variants of SARS-CoV-2 spike protein are described herein in and annotated in Table 5.

TABLE 5
Annotation of spike protein amino acid sequences provided
in the sequence listing (SEQ ID NOs: 4299-4791).

| Nucleocapsid aa ORF splits | start | end |
|---|---|---|
| Molecule/17747 [dsDNA] (Start codon) | 1 | 1 |
| Molecule/4155324 [dsDNA] (secretion signal \| | 2 | 32 |
| mu-phosphatase) | | |
| Spike S1 region variant | 33 | 705 |
| Molecule/4256240 [dsDNA] (Tag \| 10xHis(SEQ ID NO: | 706 | 715 |
| 3802)) | | |

TABLE 6
Sequences of spike protein amino acid sequences provided in Table 5.

| Nucleocapsid | Functional | nt |
|---|---|---|
| aa ORF splits | description | |
| Molecule/ | start codon | atg |
| 17747 [dsDNA] | | |
| (Start codon) | | |
| Molecule/ | secretion | ggcatcctgccaagcccaggaatgcccgccctgctgtctctggtgagcctgctgtccgt |
| 4155324 | signal | gctgctgatgggatgcgtggcagagacaggcaca (SEQ ID NO: 3803) |
| [dsDNA] | | |
| (secretion | | |
| signal\|mu- | | |
| phosphatase) | | |
| Spike S1 | spike s1 | cagtgcgtgaacctgaccacaaggacccagctgccccctgcctataccaattctttcac |
| region variant | region | acggggcgtgtactatcccgacaaggtgtttagaagctccgtgctgcactccacacagg |
| 5324 | | atctgtttctgcctttcttttctaacgtgacctggttccacgccatccacgtgtccggc |
| | | accaatggcacaaagcggttcgacaatcccgtgctgccttttaacgatggcgtgtactt |
| | | cgccagcaccgagaagtccaacatcatcagaggctggatctttggcaccacactggact |
| | | ctaagacacagagcctgctgatcgtgaacaatgccaccaacgtggtgatcaaggtgtgc |
| | | gagttccagttttgtaatgatccattcctgggcgtgtactatcacaagaacaataagtc |
| | | ctggatggagtctgagtttcgcgtgtattctagcgccaacaattgcacatttgagtacg |
| | | tgagccagcccttcctgatggacctggagggcaagcagggcaatttcaagaacctgagg |
| | | gagttcgtgtttaagaatatcgatggctacttcaagatctacagcaagcacaccccaat |
| | | caacctggtgcgcgacctgccacagggattctccgccctggagccactggtggatctgc |
| | | ccatcggcatcaacatcacccggtttcagacactgctggccctgcacagatcctacctg |
| | | acaccaggcgactcctctagoggatggaccgcaggagctgccgcctactatgtgggcta |
| | | tctgcagccccggaccttcctgctgaagtacaacgagaatggcaccatcacagacgcag |
| | | tggattgcgccctggaccccctgtctgagacaaagtgtacactgaagagctttaccgtg |
| | | gagaagggcatctatcagacaagcaatttcagggtgcagcctaccgagtccatcgtgcg |
| | | ctttcccaatatcacaaacctgtgcccttttggcgaggtgttcaacgccaccagattcg |
| | | ccagcgtgtacgcctggaatcggaagagaatctccaactgcgtggccgactattctgtg |
| | | ctgtacaactccgcctctttcagcacctttaagtgctatggcgtgtctcccacaaagct |
| | | gaatgatctgtgctttaccaacgtgtacgccgatagcttcgtgatcaggggagacgaag |
| | | tgagacagatcgcaccaggacagacaggaaagatcgcagactacaattataagctgcct |
| | | gacgatttcaccggctgcgtgatcgcctggaactccaacaatctggattctaaagtggg |
| | | cggcaactacaattatctgtacaggctgtttcgcaagtccaatctgaagccattcgagc |
| | | gggacatctccacagagatctaccaggccggctctaccccctgcaatggcgtggagggc |
| | | tttaactgttatttccctctgcagtcttacggcttccagccaacaaacggcgtgggcta |
| | | tcagccctacagagtggtggtgctgtcctttgaggaactgcacgcaccagcaacagtgt |
| | | gcggcccaaagaagtctaccaatctggtgaagaacaagtgcgtgaacttcaacttcaac |
| | | ggactgaccggcacaggcgtgctgaccgagagcaataagaagttcctgccttttcagca |
| | | gttcggcagggacatcgcagataccacagacgccgtgcgcgaccctcagaccctggaga |
| | | tcctggacatcacaccatgcagcttcggcggcgtgtccgtgatcacaccaggcaccaat |
| | | acatccaaccaggtggccgtgctgtatcaggacgtgaattgtaccgaggtgcccgtggc |
| | | aatccacgcagatcagctgacccctacatggcgggtgtactctaccggcagcaacgtgt |
| | | tccagacaaggtgccagctaccagacccagacaaactccccatctggagcaggctcc |
| | | (SEQ ID NO: 3804) |
| Molecule/ | purification | caccatcaccatcaccateaccatcaccac (SEQ ID NO: 3805) |
| 4256240 | tag | |
| [dsDNA] | | |
| (Tag\|10xHis) | | |
| (SEQ ID NO: | | |
| 3802)) | | |

In some embodiments, a variant of SARS-CoV-2 spike protein comprises at least one substitution, deletion and/or insertion of any variant of SARS-CoV-2 spike protein described herein (e.g., SEQ ID NOs 42994791).

In some embodiments, the present disclosure pertains to: A non-naturally occurring variant of the SARS-CoV-2 spike protein, wherein the amino acid sequence of the variant comprises amino acids 33-705 of any of SEQ ID NOs: 4299-4791.

In some embodiments, the present disclosure pertains to: A non-naturally occurring variant of the SARS-CoV-2 spike protein, wherein the amino acid sequence of the variant comprises any of SEQ ID NOs: 4299-4791.

In some embodiments, the present disclosure pertains to: A non-naturally occurring variant of the SARS-CoV-2 spike protein, wherein the amino acid sequence of the variant is amino acids 33-705 of any of SEQ ID NOs: 4299-4791.

In some embodiments, the present disclosure pertains to: A non-naturally occurring variant of the SARS-CoV-2 spike protein, wherein the amino acid sequence of the variant is any of SEQ ID NOs: 4299-4791.

In some embodiments, the present disclosure pertains to: A non-naturally occurring variant of the SARS-CoV-2 spike protein, wherein the amino acid sequence of the variant comprises a span of at least 15 contiguous amino acids of amino acids 33-705 of any of SEQ ID NOs: 42994791, wherein the span comprises at least one variation from the corresponding span of the wild-type SARS-CoV-2 spike protein.

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at one or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at one or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at two or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at two or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at three or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at three or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

An isolated recombinant nucleic acid encoding a non-naturally occurring variant of the SARS-CoV-2 nucleocapsid protein, wherein the nucleic acid has a sequence comprising any of SEQ ID NOs: 3806-4298, or the complementary sequence thereof.

In some embodiments, the present disclosure pertains to: A host cell comprising an isolated recombinant nucleic acid encoding a non-naturally occurring variant of the SARS-CoV-2 nucleocapsid protein, wherein the nucleic acid has a sequence comprising any of SEQ ID NOs: 3806-4298, or the complementary sequence thereof.

### Additional Variants of SARS-CoV-2 Spike Protein

In some embodiments, various additional novel variants of SARS-CoV-2 spike protein are described herein. In some embodiments, various novel variants of SARS-CoV-2 spike protein are described herein (e.g., SEQ ID NOs: 48164838).

In some embodiments, the present disclosure pertains to: A non-naturally occurring variant of the SARS-CoV-2 spike protein, wherein the amino acid sequence comprises any of the amino acid sequences set forth in SEQ ID NOs: 4816-4838.

In some embodiments, the present disclosure pertains to: A non-naturally occurring variant of the SARS-CoV-2 spike protein, wherein the amino acid sequence of the variant is any of the amino acid sequences set forth in SEQ ID NOs: 4816-4838.

In some embodiments, the present disclosure pertains to: A non-naturally occurring variant of the SARS-CoV-2 spike protein, wherein the amino acid sequence of the variant comprises amino acids 33-705 of any of SEQ ID NOs: 4816-4838.

In some embodiments, the present disclosure pertains to: A non-naturally occurring variant of the SARS-CoV-2 spike protein, wherein the amino acid sequence of the variant is amino acids 33-705 of any of SEQ ID NOs: 4816-4838.

In some embodiments, the present disclosure pertains to: A non-naturally occurring variant of the SARS-CoV-2 spike protein, wherein the amino acid sequence of the variant comprises a span of at least 15 contiguous amino acids of amino acids 33-705 of any of SEQ ID NOs: 4816-4838, wherein the span comprises at least one variation from the corresponding span of the wild-type SARS-CoV-2 spike protein.

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at one or more of: amino acids at positions 115, 200, 370, 372, 375, 376 and/or 415 and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution at one or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at two or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at two or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at three or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution at three or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 25%).

An isolated recombinant nucleic acid encoding a non-naturally occurring variant of the SARS-CoV-2 spike protein, wherein the nucleic acid has a sequence set forth in SEQ ID NOs. 4793-4815, or the complementary sequence thereof.

In some embodiments, the present disclosure pertains to: A host cell comprising an isolated recombinant nucleic acid encoding a non-naturally occurring variant of the SARS-CoV-2 spike protein, wherein the nucleic acid has a sequence set forth in SEQ ID NOs. 4793-4815, or the complementary sequence thereof.

### Variants of SARS-CoV-2 Spike Ectodomain

In some embodiments, various novel variants of SARS-CoV-2 spike ectodomain protein are described herein. In some embodiments, various novel variants of SARS-CoV-2 spike ectodomain protein are described herein in (e.g., SEQ ID NOs: 5248-5656).

In some embodiments, the present disclosure pertains to: A non-naturally occurring variant of the SARS-CoV-2 spike ectodomain protein, wherein the amino acid sequence comprises any of the amino acid sequences set forth in SEQ ID NOs: 5248-5656.

In some embodiments, the present disclosure pertains to: A non-naturally occurring variant of the SARS-CoV-2 spike protein, wherein the amino acid sequence of the variant comprises amino acids 33-1230 of any of SEQ ID NOs: 5248-5656.

In some embodiments, the present disclosure pertains to: A non-naturally occurring variant of the SARS-CoV-2 spike protein, wherein the amino acid sequence of the variant is amino acids 33-1230 of any of SEQ ID NOs: 5248-5656.

In some embodiments, the present disclosure pertains to: A non-naturally occurring variant of the SARS-CoV-2 spike protein, wherein the amino acid sequence of the variant comprises a span of at least 15 contiguous amino acids of amino acids 33-1230 of any of SEQ ID NOs: 5248-5656, wherein the span comprises at least one variation from the corresponding span of the wild-type SARS-CoV-2 spike protein.

In some embodiments, the present disclosure pertains to: A non-naturally occurring variant of the SARS-CoV-2 spike ectodomain protein, wherein the amino acid sequence of the variant is any of the amino acid sequences set forth in SEQ ID NOs: 5248-5656.

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at one or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488,491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at one or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at two or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399,401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488, 491,496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at two or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399,401, 407, 408, 409, 410, 417, 418, 419, 422, 424, 438, 439, 440, 443, 445, 461, 462, 464, 465, 466, 467, 469, 470, 471, 472, 477, 478, 479,480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution, insertion or deletion (e.g., a variation from the wild-type SARS-CoV-2 spike protein) at three or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

In some embodiments, the present disclosure pertains to: A non-naturally occurring variant of the SARS-CoV-2 spike protein, wherein the amino acid sequence of the variant has a substitution (e.g., a variation from the wild-type SARS-CoV-2 spike protein) at three or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

An isolated recombinant nucleic acid encoding a non-naturally occurring variant of the SARS-CoV-2 spike protein, wherein the nucleic acid has a sequence set forth in SEQ ID NOs. 4839-5247, or the complementary sequence thereof.

In some embodiments, the present disclosure pertains to: A host cell comprising an isolated recombinant nucleic acid encoding a non-naturally occurring variant of the SARS-CoV-2 spike protein, wherein the nucleic acid has a sequence set forth in SEQ ID NOs. 4839-5247, or the complementary sequence thereof.

### Amino Acid Substitution in Variants of SARS-CoV-2 Spike Protein

In some embodiments, the present disclosure pertains to: A non-naturally occurring variant of the SARS-CoV-2 spike protein, wherein the amino acid sequence of the variant comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the following mutations V36I, S46E, S46K, S46N, S46Q, V47W, H49V, S501, S50L, T63K, T63L, W64Q, W64R, I68A, I68C, I68D, I68E, I68G, I68H, I68K, I68L, I68M, I68N, I68Q, I68R, I68S, I68W, I68Y, V70G, S71C, S71E, S71K, S71Q, S71R, N81F, N81V, N81Y, L84V, F92S, F92V, S94A, S94M, S94N, T951, T95V, K971, S98D, I101F, T108A, T108I, T108N, T108V, S112G, Q115D, Q115E, A123E, A123N, N125F, N125K, N125M, N125Q, N125Y, K129I, K129V, E132F, E132R, E132Y, C136F, C136H, C136M, C136S, C136T, C136V, C136Y, D138R, F140H, H146E, H146F, H146L, H146Q, M153P, S155E, S155K, V159K, S161D, T167K, V171H, V171Y, S172Q, S172V, S172W, M177G, M177K, M177S, M177T, D178Q, G184D, F186W, K187H, N188L, N188W, F192Y, K202Y, S205M, K206R, H207P, P209E, V213W, V213Y, S221L, A222P, P225C, L226A, L226E, L226H, L226K, L226Q, L226R, L226T, D228I, T236I, R237I, R237Y, Q239A, Q239E, Q239L, Q239S, T240F, T240P, T240Y, L241F, L241I, L241V, L241Y, H245G, H245S, L249K, L249N, L249Q, G252E, G252I, G252R, G252T, G252V, G257L, W258D, W258E, A260E, A260M, A262I, A264Q, T284F, T284W, A292N, A292S, A292T, A292Y, E298K, T299I, T302L, S316T, Q321E, E324C, E324F, E324I, E324T, E324V, S325V, F329Y, L335A, L335C, L335E, L335F, L335H, L335K, L335N, L335P, L335Q, L335R, L335S, L335T, L335Y, F338L, G339D, G339W, A344E, T345P, V350S, N354I, N354R, N354V, K356R, V367E, L368W, S375A, S375T, F377I, K378Q, C379W, T393V, S399D, S399E, S399I, S399L, S399Q, S399R, S399V, K417A, K417E, K417H, K417I, K417N, K417P, K417T, K417V, A419H, D420L, K424V, C432I, N439S, S443A, V445P, S459D, S459L, S459M, N460H, L461I, L461T, F464V, F464W, D467F, D467Y, T470E, T470I, T470K, T470R, T470V, I472A, I472C, I472D, I472E, I472F, I472H, I472K, I472M, I472N, I472Q, I472W, I472Y, A475S, S477G, S477K, S477L, S477N, C480A, C480S, C480T, C480Y, G482E, G482M, G482Q, E484P, N487L, N487P, C488A, C488Q, Y489D, Y489H, Y489K, Y489N, Y489Q, F490C, F490N, F490P, F490T, G496T, G496V, N501F, N501Y, V503A, V503D, V503E, V503K, V503P, V503R, V503T, G504D, G504E, G504N, G504Q, G504R, G504T, Y505D, Y505E, Y505H, Y505K, Y505N, Y505Q, Y505R, S514E, S514I, S514L, L517C, L517D, L517E, L517N, L517P, L517R, L517T, L518E, L518H, L518K, L518Q, L518T, H519D, H519F, H519L, A522H, G526P, K528R, S530T, V534R, V534T, K537E, L546E, V55I F, K557R, G566A, G566L, G566M, G566Q, G566S, G566T, R567L, D568K, D568S, D568T, D568W, 1569D, 1569P, D571G, T573L, D574R, L582P, L585I, T588I, T588V, F592A, F592C, F592D, F592E, F592H, F592K, F592M, F592N, F592Q, F592R, F592S, F592T, F592Y, V595K, S596F, S596L, S596Y, G601A, G601D, G601N, G601Q, G601S, N603R, V608F, P621Q, V622A, V622C, V622D, V622E, V622K, V622N, V622S, V622T, V622W, A623Q, I624Q, H625F, D627Q, D627R, T630R, V635A, V635D, V635E, V635K, V635M, V635N, V635Q, V635T, Y636E, S637A, S637N, V642Y, I651V, A653F, A653I, A653V, H655F, H655P, H655Y, 1666V, A668N, S673T, T678R, R682S, R683G, R685G, F32G, F32K, F32S, F32T, F32V, T33K, T33R, K41D, K41E, K41M, V421, S46D, S46N, V471, V47T, S50D, S50G, S50L, S50M, S50N, I68L, I68W, S71K, K77V, R78A, R78D, R78E, R78N, R78Q, V831, L84I, N87G, N87K, N87R, V90I, S94A, S94N, S94T, T951, I100V, I101V, T108S, S112G, S112N, K113T, T114S, K129R, E132N, E132Y, Q134N, F135L, N137A, N137D, N137E, N137K, N137Q, N137Y, D138N, F140M, I141F, G142A, G142S, G142T, G142V, M153R, S155A, S155D, S155E, S155F, S155I, S155K, S155L, S155N, S155Q, S155R, S155T, S155V, S155Y, F157W, V159W, V171A, V171K, V171N, V171S, V171T, S172E, S172H, S172K, S172P, S172Q, Q173D, Q173K, Q173R, P174A, P174D, P174S, F175L, F175N, L176A, L176D, L176E, L176H, L176K, L176M, L176N, L176P, L176Q, L176R, L176S, L176T, M177F, M177I, M177L, M177P, M177T, M177Y, L179T, L179V, E180A, E180D, E180G, E180N, E180S, G181E, G181P, K187H, N188D, N188H, N188Q, N188T, N188W, K202Y, K206G, K206T, H207F, H207W, H207Y, T208E, T208Q, A222P, A222V, E224K, E224P, E224Q, E224R, L226D, L226I, L226K, L226N, L226S, L226T, V227A, V227F, V227L, V227M, V227T, V227W, D228E, D228K, D228N, D228S, D228T, D228V, R237H, L241F, L241I, A243S, H245S, L249R, T259D, T259E, T259G, T259N, T259Q, T259R, T259S, G261E, G261F, G261S, A262S, Y266F, Y266W, Q271E, Q271K, R273T, L276M, Y279F, N280D, T284Y, A292D, A292G, A292N, A292S, A292T, L293F, L293H, L293Q, L293Y, S297A, T299A, T299L, T299V, T302K, T302R, T302S, T302Y, E324Y, S325E, L335A, L335C, L335E, G339D, T345P, A352N, N354E, N354R, K356H, K356Q, K356Y, K378Q, S399I, S399V, D405S, K417E, K417P, K417T, K444T, L461M, I472A, I472Q, S477C, S514K, K529D, S530P, K537E, K537Q, K537R, K537V, N540D, F541Y, L546R, T547K, E554D, E554P, E554S, N556S, 1569A, 1569L, I569T, 1569V, A570S, T572A, T572C, T572F, T572I, T572L, T572M, T572N, T572Q, T572R, T572S, T572V, T573L, A575S, Q580T, L582G, L582N, L582S, D586C, T588Q, T588R, T588S, T588V, 1598V, N606S, Q607A, Q607E, E619D, E619H, V622A, V622D, V622E, V622G, V622H, V622K, V622L, V622N, V622P, V622Q, V622R, V622S, V622T, V622Y, A623D, A623G, A623S, A623T, A626S, D627N, T630R, R646Q, H655Y, A668N, R682S, R683G, A684S, R685G, A688S, Q690D, Q690E, Q690K, Q690N, 1693V, S704P, S704T, A706P, S711E, S711N, A713S, T719A, T719K, T719L, T719M, T719N, T719Q, T719S, T719V, T719Y, T723Q, 1726F, 1726V, 1726Y, L727I, L727M, T732A, T732D, T732P, T732Q, T734V, T739A, T739E, T739S, T747E, T747P, T747S, E748R, S750L, L754K, L754S, T761D, T761S, N764E, T768A, T768E, T768H, T768N, T768Q, T768R, T768S, V772A, V772D, V772E, V772I, V772K, V772L, V772M, V772N, V772Q, V772R, V772S, Q774E, T778L, Q779L, E780N, 1788M, F817A, V826A, V826D, V826E, V826I, V826L, V826T, V826W, A831L, 1834V, G838K, D839E, D839K, L841M, L841T, 1844Q, 1844V, A845D, A845N, A845S, F855Y, L858I, T859M, Q872A, Q872M, S875A, A876S, L878I, L878V, A879S, 1882A, S884A, S884G, S884T, Q895V, T912Q, K921Q, L922I, L922K, L922M, L922Q, S929A, S929D, S929E, S929K, S929N, S929Q, S929R, S929T, G932K, G932S, K933A, K933H, K933N, K933Q, D936E, D936K, S937G, S939R, S939T, S940T, A942S, S943N, S943T, G946S, N953E, N953K, N953S, N955Q, N960A, N960D, N960E, N960H, N960I, N960K, N960M, N960Q, N960S, N960T, T961E, L977I, L981Y, S982N, K986P, K986Q, K986R, V987P, E990Q, V991A, 1993V, A1016T, N1023Q, T1027E, L1034K, K1038N, V1040Y, D1041G, K1045N, S1055A, V10601, A1070T, E1072F, A1080G, D1084E, H1088F, H1088W, H1088Y, H1101A, H101E, H1101K, H1101M, H1101Q, H1101R, HI 101S, H1101Y, F1103Y, V1104I, Q1113R, I1114L, I1114N, I1114P, I1114S, I1114T, I1114V, N1125S, I1130F, I1130Y V11331 Q115L, Q115V, Y200F, Y2001, Y200V, N370R, A372P, S375N, S375R, S375V, S375W, T376A, T376C, T376E, T376L, T376M, T376Q, T376S, T415H, T415P, T415S, T415W, T415Y K41N, Q15A, Q115E, Q115M, Q115P, Q115T, L226A, L226E, L226Q, L226W, N282T, N282Y, R319Q, P330T, N331A, N331D, N331P, N331R, N331V, 1332V, F3381, F338L, F338M, F338Y, V3411, V341Y, A348C, A348G, A348P, A348S, A348V, S349P, Y35I A, Y35I S, A352D, A352Q, A352S, W353F, W353P, W353Y, R357V, 1358V, S359T, N360G, N360K, V3621, A363F, D364N, Y365F, S366R, V367A, V367E, V367G, V3671, V367K, V367L, V367N, V367Q, V367R, V367S, V367T, Y369F, N370A, N370D, N370E, N370K, N370L, N370Q, N370S, N370T, S371T, A372C, A372D, A372E, A372G, A372K, A372P, A372Q, A372S, A372T, S373T, S375A, S375C, S375D, S375E, S375K, S375M, S375Q, S375T, S375V, T376S, T385S, N388D, N388T, D389H, L390Q, T393P, T393V, S399K, S399L, S399N, S399Q, S399R, S399T, V401L, V407I, V407L, R408K, Q409E, 1410V, K417A, K417C, K417D, K417H, K417I, K417N, K417Q, K417R, K417S, K417V, 1418V, A419H, A419R, N422T, N422V, K424L, K424N, K424Q, K424R, K424T, K424V, S438A, N439A, N439C, N439D, N439E, N439G, N439H, N439I, N439K, N439Q, N439R, N439S, N439V, N440D, N440E, N440G, N440K, N440L, N440Q, N440S, N440T, S443A, S443C, V445A, V445C, V445D, V445E, V445I, V445K, V445L, V445M, V445N, V445P, V445Q, V445R, L461F, L461R, K462A, K462C, K462D, K462E, K462G, K462N, K462Q, K462R, K462S, F464V, F464Y, E465A, E465G, E465K, E465R, E465S, R466D, R466F, R4661, R466K, R466L, R466N, R466Q, R466S, R466V, D467F, D467N, D467Y, S469A, S469C, S469D, S469E, S469G, S469K, S469L, S469N, S469Q, S469T, T470A, T470C, T470D, T470E, T470F, T470G, T470H, T470K, T470L, T470M, T470N, T470P, T470Q, T470R, T470S, T470V, T470W, T470Y, E471A, E471C, E471D, E471G, E4711, E471K, E471L, E471M, E471N, E471Q, E471R, E471S, E471T, E471V, I472C, I472D, I472E, I472F, I472G, I472H, I472K, I472M, I472N, I472R, I472S, I472T, I472V, I472W, I472Y, S477A, S477G, S477K, T478S, P479V, C480A, C480G, C480Q, C480S, C480T, C480Y, N481T, G482A, G482S, C488A, C488E, C488H, C488Q, C488R, C488S, C488W, C488Y, P491A, G496D, G496T, G496V, V5121, S514A, S514E, S514F, S514I, S514L, S514N, S514Q, S514T, S514V, L518A, L518D, L5181, L518K, L518N, L518Q, L518R, L518S, L518T, L518V, H519A, H519C, H519D, H519E, H519F, H519G, H519K, H519L, H519M, H519N, H519P, H519Q, H519R, H519S, H519W, H519Y, A520Y, A522C, A522H, K528R, L546E, L546H, L546V, K558Q, 1569M, T588E, T5881, T588K, S591P, M697A, M697C, M697E, M697Q, M697R, M697S, M697V, A701D, E702T, E702V, S704C, S704E, S7041, S704K, S704M, S704N, S704Q, S704R, S704V, V705I, S711A, S711H, Q787H, M869L, M869Q, A890F, A890R, A890Y, Q8951, Q895T, A899D, Y904W, V963I, K964A, S968Y, N969K, N969R, N969S, N969T, N978W, D979A, D979E, D979N, D979S, L984A, L984I, L984S, L984V, D985A, D985E, D985N, D985P, D985S, E988A, E988D, E988Q, E988S, S1030Q, V1040Q, D1041H, K1045E, K1045P, K1045Q, K1045R, K1045T, Y1047F, E1072A, E1072C, E1072D, E1072G, E1072H, E1072M, E1072N, E1072Q, E1072R, E1072S, E1072T, E1072W, E1072Y, E1144R and/or L1145R relative to the wild-type spike protein amino acid sequence (SEQ ID NO: 2596).

### Variants of SARS-CoV-2 Spike Protein RBD (Receptor Binding Domain)

In some embodiments, various novel variants of SARS-CoV-2 spike protein receptor binding domain (RBD) are described herein. In some embodiments, various novel variants of SARS-CoV-2 spike protein receptor binding domain (RBD) are described herein and annotated in Table 7. In some embodiments, a variant of SARS-CoV-2 spike protein receptor binding domain (RBD) comprises at least one substitution, deletion and/or insertion of any variant of SARS-CoV-2 spike protein receptor binding domain (RBD) described herein, e.g., in SEQ ID NOs: 2494-2592.

TABLE 7
Annotation of spike protein RBD amino acid sequences provided
in the sequence listing SEQ ID NOs: 2494-2592.

| RBD aa ORF | start | end |
|---|---|---|
| Molecule/17747 [dsDNA] (Start codon) | 1 | 1 |
| Molecule/4155324 [dsDNA] (secretion signal \| | 2 | 32 |
| mu-phosphatase) | | |
| RBD variant | 33 | 236 |
| Molecule/4155312 [dsDNA] (Tag \| 8xHis \| (SEQ | 237 | 244 |
| ID NO: 2598) H. sapiens recode 1) | | |

In some embodiments, the present disclosure pertains to: A non-naturally occurring variant of the SARS-CoV-2 spike protein RBD, wherein the amino acid sequence of the variant comprises amino acids 33-236 of any of SEQ ID NOs: 2494 to 2592.

In some embodiments, the present disclosure pertains to: A non-naturally occurring variant of the SARS-CoV-2 spike protein RBD, wherein the amino acid sequence of the variant comprises any of SEQ ID NOs: 2494 to 2592.

In some embodiments, the present disclosure pertains to: A non-naturally occurring variant of the SARS-CoV-2 spike protein RBD, wherein the amino acid sequence of the variant is amino acids 33-236 of any of SEQ ID NOs: 2494 to 2592.

In some embodiments, the present disclosure pertains to: A non-naturally occurring variant of the SARS-CoV-2 spike protein RBD, wherein the amino acid sequence of the variant is any of SEQ ID NOs: 2494 to 2592.

In some embodiments, the present disclosure pertains to: A non-naturally occurring variant of the SARS-CoV-2 spike protein RBD, wherein the amino acid sequence of the variant comprises amino acids 33-236 of any of SEQ ID NOs: 2494 to 2592, and wherein the span comprises at least one variation from the corresponding span of the wild-type SARS-CoV-2 spike protein RBD protein.

In some embodiments, the present disclosure pertains to: A non-naturally occurring variant of the SARS-CoV-2 spike protein RBD, wherein the amino acid sequence of the variant has a substitution, insertion or deletion (e.g., a variation from the wild-type SARS-CoV-2 spike protein RBD) at one or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acids at positions 1 to 327 and a deletion of amino acids at positions 532 to 1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution (e.g., a variation from the wild-type SARS-CoV-2 spike protein RBD) at one or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acids at positions 1 to 327 and a deletion of amino acids at positions 532 to 1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution, insertion or deletion (e.g., a variation from the wild-type SARS-CoV-2 spike protein RBD) at two or more of: amino acids at positions 335, 339, 340, 356, 357, 362, 366, 368, 372, 393, 394, 399, 402, 403, 404, 405, 406, 410, 414,417, 438, 439, 450, 477, 478, 483, 484, 486, 489, 490, 501, 502, or 504, and a deletion of amino acids at positions 1 to 327 and a deletion of amino acids at positions 532 to 1273, relative to the wild-type spike protein amino acid sequence (SEQ ID NO: 2596).

In some embodiments, the present disclosure pertains to: A non-naturally occurring variant of the SARS-CoV-2 spike protein RBD, wherein the amino acid sequence of the variant has a substitution (e.g., a variation from the wild-type SARS-CoV-2 spike protein RBD) at two or more of: amino acids at positions 335, 339, 340, 356, 357, 362, 366, 368, 372, 393, 394, 399, 402, 403, 404, 405, 406,410, 414, 417, 438, 439, 450, 477, 478, 483, 484, 486, 489, 490, 501, 502, or 504, and a deletion of amino acids at positions 1 to 327 and a deletion of amino acids at positions 532 to 1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution, insertion or deletion (e.g., a variation from the wild-type SARS-CoV-2 spike protein RBD) at three or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489,490, 501, 502, or 504, and a deletion of amino acids at positions 1 to 327 and a deletion of amino acids at positions 532 to 1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution (e.g., a variation from the wild-type SARS-CoV-2 spike protein RBD) at three or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of amino acids at positions 1 to 327 and a deletion of amino acids at positions 532 to 1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the variant is described in Table 7 herein.

An isolated recombinant nucleic acid encoding a non-naturally occurring variant of the SARS-CoV-2 spike protein RBD (receptor binding domain), wherein the nucleic acid has a sequence comprising any of SEQ ID NOs: 2395 to 2493, or the complementary sequence thereof.

In some embodiments, the present disclosure pertains to: A host cell comprising an isolated recombinant nucleic acid encoding a non-naturally occurring variant of the SARS-CoV-2 spike protein RBD (receptor binding domain), wherein the nucleic acid has a sequence comprising any of SEQ ID NOs: 2395 to 2493, or the complementary sequence thereof.

### Additional Variants of SARS-CoV-2 Spike Protein RBD (Receptor Binding Domain)

In some embodiments, various novel variants of SARS-CoV-2 spike protein receptor binding domain (RBD) are described herein. In some embodiments, various novel variants of SARS-CoV-2 spike protein receptor binding domain (RBD) are described herein and annotated in SEQ ID NOs: 5957-6256.

In some embodiments, the present disclosure pertains to: A non-naturally occurring variant of the SARS-CoV-2 spike protein RBD (receptor binding domain), wherein the variant comprises an amino acid sequence listed in SEQ ID NOs: 5957-6256.

In some embodiments, the present disclosure pertains to: A non-naturally occurring variant of the SARS-CoV-2 spike protein RBD (receptor binding domain), wherein the variant is set forth in SEQ ID NOs: 5957-6256.

In some embodiments, the present disclosure pertains to: A non-naturally occurring variant of the SARS-CoV-2 spike protein RBD, wherein the amino acid sequence of the variant has a substitution, insertion or deletion (e.g., a variation from the wild-type SARS-CoV-2 spike protein RBD) at one or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution (e.g., a variation from the wild-type SARS-CoV-2 spike protein RBD) at one or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407,408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution, insertion or deletion (e.g., a variation from the wild-type SARS-CoV-2 spike protein RBD) at two or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 25%).

In some embodiments, the present disclosure pertains to: A non-naturally occurring variant of the SARS-CoV-2 spike protein RBD, wherein the amino acid sequence of the variant has a substitution (e.g., a variation from the wild-type SARS-CoV-2 spike protein RBD) at two or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution, insertion or deletion (e.g., a variation from the wild-type SARS-CoV-2 spike protein RBD) at three or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

In some embodiments, the present disclosure pertains to: A non-naturally occurring variant of the SARS-CoV-2 spike protein RBD (receptor binding domain), wherein the amino acid sequence of the variant has a substitution (e.g., a variation from the wild-type SARS-CoV-2 spike protein RBD) at three or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407,408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

An isolated recombinant nucleic acid encoding a non-naturally occurring variant of the SARS-CoV-2 spike protein RBD (receptor binding domain), wherein the nucleic acid has a sequence comprising of any of SEQ ID NOs: 5657-5956, or the complementary sequence thereof.

In some embodiments, the present disclosure pertains to: A host cell comprising an isolated recombinant nucleic acid encoding a non-naturally occurring variant of the SARS-CoV-2 spike protein RBD (receptor binding domain), wherein the nucleic acid has a sequence comprising of any of SEQ ID NOs: 5657-5956, or the complementary sequence thereof.

### Amino Acid Substitutions in Variants of SARS-CoV-2 Spike Protein RBD (Receptor Binding Domain)

In some embodiments, the present disclosure pertains to: A non-naturally occurring variant of the SARS-CoV-2 spike protein, wherein the amino acid sequence of the variant comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the following mutations L335R, L335V, G339D, G339H, G339N, E340K, E340R, K356L, K356T, K356V, R357K, R357V, V3621, S366T, L368F, A372C, A372D, A372F, A372G, A372H, A372N, A372Q, A372S, A372T, A372V, T393S, N394S, S399Y, 1402V, R403P, G404F, D405S, E406D, 1410V, Q414E, K417A, K417E, K417F, K417L, K417T, K417V, S438A, N439A, N439S, N450Q, N450S, S477A, S477G, S477Q, T478A, T478D, T478E, T4781, T478K, T478N, T478Q, T478S, V483A, V483Q, V483R, E484A, E484K, E484P, E484Q, E484R, E484S, E484T, E484V, E484Y, F4861, F486L, F486P, F486T, F486V, F486Y, Y489R, F490A, F490D, F490E, F490G, F490H, F490K, F490L, F490M, F490N, F490P, F490R, F490S, F490T, F490V, F490W, F490Y, N501D, N501E, N501L, G502P, G504A, G504D, G504E, P330T, N331A, N331D, N331P, N331R, N331V, 1332V, F3381, F338L, F338M, F338Y, V3411, V341Y, A348C, A348G, A348P, A348S, A348V, S349P, Y351A, Y351S, A352D, A352N, A352Q, A352S, W353F, W353P, W353Y, 1358F, 1358L, 1358M, 1358V, 1358W, 1358Y, V362K, V362Q, V362S, V362T, V362W, V362Y, A363F, A363W, D364N, Y365F, Y3651, Y365L, Y365M, Y365V, Y365W, S366R, V367A, V367E, V367G, V3671, V367K, V367L, V367N, V367Q, V367R, V367S, V367T, N370A, N370D, N370E, N370K, N370L, N370Q, N370S, N370T, S371T, S373T, T376S, P384A, P384K, P384M, C391N, F392A, F392V, F392W, T393P, T393V, N394T, V395I, S399I, S399K, S399L, S399N, S399Q, S399R, S399T, S399V, V401L, E406H, E406Q, V4071, V407L, R408K, Q409E, 1418V, A419H, A419R, N422T, N422V, K424L, K424N, K424Q, K424R, K424T, K424V, I434F, N439C, N439D, N439E, N439G, N439H, N439I, N439K, N439Q, N439R, N439V, N440D, N440E, N440G, N440K, N440L, N440Q, N440S, N440T, S443A, S443C, V445A, V445C, V445D, V445E, V4451, V445K, V445L, V445M, V445N, V445P, V445Q, V445R, Y453R, F456H, L461F, L461R, K462A, K462C, K462D, K462E, K462G, K462N, K462Q, K462R, K462S, F464V, F464Y, E465A, E465G, E465K, E465R, E465S, R466D, R466F, R4661, R466K, R466L, R466N, R466Q, R466S, R466V, D467F, D467N, D467Y, S469A, S469C, S469D, S469E, S469G, S469K, S469L, S469N, S469Q, S469T, T470A, T470C, T470D, T470E, T470F, T470G, T470H, T470K, T470L, T470M, T470N, T470P, T470Q, T470R, T470S, T470V, T470W, T470Y, E471A, E471C, E471D, E471G, E4711, E471K, E471L, E471M, E471N, E471Q, E471R, E471S, E471T, E471V, I472A, I472C, I472D, I472E, I472F, I472G, I472H, I472K, I472M, I472N, I472Q, I472R, I472S, I472T, I472V, I472W, I472Y, P479V, C480A, C480G, C480Q, C480S, C480T, C480Y, N48 IT, G482A, G482S, C488A, C488E, C488H, C488Q, C488R, C488S, C488W, C488Y, P491A, G496A, G496D, G496N, G496S, G496T, G496V, G504K, G504M, G504N, Y508H, V5121, S514A, S514E, S514F, S514I, S514K, S514L, S514N, S514Q, S514T, S514V, L518A, L518D, L5181, L518K, L518N, L518Q, L518R, L518S, L518T, L518V, H519A, H519C, H519D, H519E, H519F, H519G, H519K, H519L, H519M, H519N, H519P, H519Q, H519R, H519S, H519W, H519Y, and/or A520Y relative to the wild-type spike protein amino acid sequence (SEQ ID NO: 2596).

### Vaccine Compositions

In certain aspects, provided herein are compositions (e.g., vaccine compositions) comprising one or more (e.g., one, two, three, etc.) of the variants provided herein and/or one or more of the nucleic acids provided herein. In some embodiments, the composition comprises a nucleocapsid protein variant. In some embodiments, the composition comprises a spike protein variant. In some embodiments, the composition comprises a spike protein RBD variant. In some embodiments, the composition comprises a nucleocapsid protein variant and a spike protein variant. In some embodiments, the composition comprises a nucleocapsid protein variant and a spike protein RBD variant. In certain embodiments, the composition comprises a spike protein variant and a spike protein RBD variant. In some embodiments, the composition comprises a nucleocapsid protein variant, a spike protein variant and a spike protein RBD variant.

In certain embodiments, the compositions (e.g., vaccine compositions disclosed herein) disclosed herein further comprise and/or are administered with an adjuvant. Adjuvants suitable for use in animals include, but are not limited to, Freund's complete or incomplete adjuvants, Sigma Adjuvant System (SAS), and Ribi adjuvants. Adjuvants suitable for use in humans include, but are not limited to, MF59 (an oil-in-water emulsion adjuvant); Montanide ISA 51 or 720 (a mineral oil-based or metabolizable oil-based adjuvant): aluminum hydroxide, -phosphate, or -oxide; HAVLOGEN® (an acrylic acid polymer-based adjuvant, Intervet Inc., Millsboro, Del.); polyacrylic acids; oil-in-water or water-in-oil emulsion based on, for example a mineral oil, such as BAYOL™ or MARCOL™ (Esso Imperial Oil Limited, Canada), or a vegetable oil such as vitamin E acetate; saponins; and Onchocerca volvulus activation-associated protein-1 (Ov ASP-1) (see US 20060039921, which is incorporated by reference herein for all it discloses regarding Ov ASP-1 adjuvants). However, components with adjuvant activity are widely known and, generally, any adjuvant may be utilized.

In some embodiments, the present disclosure pertains to a vaccine comprising a nucleic acid encoding a variant disclosed herein (e.g., an mRNA encoding a variant disclosed herein). The vaccine may further comprise any one or more of: solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotide, primary construct, hyaluronidase, polymer based-self assembly nanoparticles, inorganic nanoparticles, semi-conductive and metallic nanoparticles, gels, hydrogels, molded nanoparticles, molded microparticles, NanoJackets, NanoLiposomes, or nanoparticle mimics and/or combinations thereof.

The polynucleotide (e.g., mRNA) of the invention can be formulated using one or more liposomes, lipoplexes, or lipid nanoparticles. In one embodiment, pharmaceutical compositions of polynucleotide include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.). Additional mRNA vaccine formulations can be found in U.S. Pat. No. 9,675,668, hereby incorporated by reference in its entirety.

Vaccines and/or immunogenic compositions according to the various embodiments disclosed herein can be prepared and/or marketed in the form of a liquid, frozen suspension, or in a lyophilized form. Typically, vaccines and/or immunogenic compositions prepared according to the present disclosure contain a pharmaceutically acceptable carrier or diluent customarily used for such compositions. Carriers include, but are not limited to, stabilizers, preservatives, and buffers. Suitable stabilizers are, for example SPGA, Tween compositions (such as are available from A.G. Scientific, Inc., San Diego, Calif.), carbohydrates (such as sorbitol, mannitol, starch, sucrose, dextran, glutamate, or glucose), proteins (such as dried milk serum, albumin, or casein), or degradation products thereof. Examples of suitable buffers include alkali metal phosphates. Suitable preservatives include thimerosal, merthiolate, and gentamicin. Diluents include water, aqueous buffer (such as buffered saline), alcohols, and polyols (such as glycerol).

Formulations disclosed herein may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins. Baltimore, Md., 2006; incorporated herein by reference in its entirety).

Further, in various formulations of the proteins and/or compositions, suitable excipients, stabilizers, and the like may be added as are known by persons of ordinary skill in the art. Formulations of the compositions described herein may be prepared by any method known or hereafter developed in the art of pharmacology. Such preparatory methods may include the step of associating the active ingredient with an excipient and/or one or more other accessory ingredients.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient.

### Diagnostic Test Kits

In certain aspects, provided herein are diagnostic test kits comprising one or more of the variant proteins disclosed herein. In certain embodiments, the diagnostic test kits provided herein are sensitive, specific, and/or inexpensive high throughput diagnostic test kits for the diagnosis of SARS-CoV-2 exposure. In some embodiments, the present disclosure pertains to a diagnostic test kit useful for determining if a subject has been exposed and/or is infected with SARS-CoV-2. The present disclosure also pertains to: A system or kit for the serological detection of exposure to and/or infection by SARS-CoV-2.

In some embodiments, the present disclosure pertains to a diagnostic test kit comprising a variant of SARS-CoV-2 nucleocapsid protein, spike protein and/or spike protein RBD (receptor binding domain). In some embodiments, the variant of SARS-CoV-2 nucleocapsid protein, spike protein and/or spike protein receptor binding domain (RBD) is described herein.

The present disclosure also pertains to the use of a variant of SARS-CoV-2 nucleocapsid protein, spike protein and/or spike protein RBD (receptor binding domain), which are strongly reactive with, and sensitive to an antibody to SARS-CoV-2 in a patient sample. In some embodiments, a variant of SARS-CoV-2 nucleocapsid protein, spike protein and/or spike protein receptor binding domain (RBD) can be used in any type of serological assay or platform, now known or later developed, to screen for the presence of antibody to SARS-CoV-2 and to determine if a subject has had an infection by and/or exposure to SARS-CoV-2. In some embodiments, a variant of SARS-CoV-2 nucleocapsid protein, spike protein and/or spike protein receptor binding domain (RBD) can also be used to test for and monitor humoral responses to vaccines and immunomodulatory drugs, thus, being useful for the development of treatment and preventative agents for SARS-CoV-2.

The current disclosure provides compositions, methods, devices, and kits for detecting the exposure to, and infection by, SARS-CoV-2. Specifically, the current disclosure allows for the rapid differential serological detection of exposure to, and infection by SARS-CoV-2.

The compositions, methods, devices, and kits for detection of exposure to, and infection by SARS-CoV-2 comprise a variant of SARS-CoV-2 nucleocapsid protein, spike protein and/or spike protein RBD (receptor binding domain), isolated and non-isolated, which are strongly reactive with, and specific for SARS-CoV-2, i.e., reactive and specific epitopes of antibody to SARS-CoV-2.

In some embodiments, a diagnostic test kit is a serological assay.

In some embodiments, the present disclosure pertains to: A method for the serological detection of exposure to and/or infection by SARS-CoV-2, comprising the use of a variant of SARS-CoV-2 nucleocapsid protein, a variant of the spike protein and/or a variant of the spike protein receptor binding domain (RBD) which are reactive with, and specific for SARS-CoV-2 antibody.

In some embodiments, the present disclosure pertains to: A method for the serological detection of exposure to and/or infection by SARS-CoV-2 in a sample, comprises: contacting the sample with a variant of SARS-CoV-2 nucleocapsid protein, spike protein and/or spike protein receptor binding domain (RBD) or a variant of SARS-CoV-2 nucleocapsid protein, spike protein and/or spike protein receptor binding domain (RBD) which are reactive with, and specific for a SARS-CoV-2 antibody; and detecting the binding between the antibody in the sample and the variant.

In some embodiments, the present disclosure pertains to: A method for the serological detection of exposure to and/or infection by SARS-CoV-2 in a sample, comprises: contacting the sample with a microarray comprising a variant of SARS-CoV-2 nucleocapsid protein, spike protein and/or spike protein receptor binding domain (RBD) which are reactive with, and specific for SARS-CoV-2 antibody.

In certain embodiments, the detecting step comprises performing an ELISA assay. In other embodiments, the detecting step comprises performing a lateral flow immunoassay. In other embodiments, the detecting step comprises performing an agglutination assay. In other embodiments, the detecting step comprises spinning the sample in an analytical or centrifugal rotor. In other embodiments, the detecting step comprises analyzing the sample using a Western blot, a slot blot, or a dot blot. In still other embodiments, the detecting step comprises analyzing the sample with an electrochemical sensor, an optical sensor, or an opto electronic sensor. In certain embodiments, the detecting step comprises performing a wavelength shift assay.

In certain embodiments, the diagnostic test kit comprises a variant of SARS-CoV-2 nucleocapsid protein, spike protein and/or spike protein receptor binding domain (RBD) attached to or immobilized upon a solid support. In one embodiment, the variant is attached to the solid support through a metallic (e.g., gold) nanolayer. In certain embodiments, the solid support is a bead or plurality of beads (e.g., a colloidal particle, a metallic nanoparticle or nanoshell, or a latex bead), a flow path in a lateral flow immunoassay device (e.g., a porous membrane), a flow path in an analytical or centrifugal rotor, a blot (e.g., Western blot, a slot blot, or dot blot), or a tube or a well (e.g., in a plate suitable for an ELISA assay or microarray). In certain embodiments, the solid support comprises metal, glass, a cellulose-based material (e.g., nitrocellulose), or a polymer (e.g., polystyrene, polyethylene, polypropylene, polyester, nylon, or polysulfone).

The present disclosure also pertains to the use of a variant of SARS-CoV-2 nucleocapsid protein, spike protein and/or spike protein receptor binding domain (RBD) which are strongly reactive with, and sensitive to an antibody to SARS-CoV-2 in a patient sample. In some embodiments, a variant of SARS-CoV-2 nucleocapsid protein, spike protein and/or spike protein receptor binding domain (RBD) can be used in any type of serological assay or platform, now known or later developed, to screen for the presence of antibody to SARS-CoV-2 and to determine if a subject has had an infection by and/or exposure to SARS-CoV-2. In some embodiments, a variant of SARS-CoV-2 nucleocapsid protein, spike Protein and/or spike protein receptor binding domain (RBD) can also be used to test for and monitor humoral responses to vaccines and immunomodulatory drugs, thus, being useful for the development of treatment and preventative agents for SARS-CoV-2.

As one of ordinary skill in the art would know, a number of different conventional assays are useful for detecting formation of a complex comprising an antibody and a viral protein or variant thereof. For example, the detecting step can comprise performing an ELISA assay, performing an immunofluorescence assay, performing a lateral flow immunoassay, performing an agglutination assay, performing a wavelength shift assay, performing a Western blot, slot blot, or dot blot, analyzing the sample in an analytical or centrifugal rotor, or analyzing the sample with an electrochemical, optical, or opto-electronic sensor. These different assays are described herein and/or are well known to those skilled in the art.

Thus, the variant of SARS-CoV-2 nucleocapsid protein, spike protein and/or spike protein receptor binding domain (RBD) of the present disclosure can be used in any assay, format or platform for antibody detection including but not limited to ELISA, Luminex, Western blot assays, and spotted arrays, as well as those platforms that are later developed.

In certain embodiments, the assay comprises: immobilizing the antibody(s) in the sample; adding a variant of SARS-CoV-2 nucleocapsid protein, spike protein and/or spike protein RBD (receptor binding domain): and detecting the degree of antibody bound to the variant, e.g., by the variant being labeled or by adding a labeled substance, such as a labeled binding partner (e.g., streptavidin-HRP or streptavidin-colloidal gold complex) or a labeled antibody which specifically recognizes the variant of SARS-CoV-2 nucleocapsid protein, spike protein and/or spike protein RBD (receptor binding domain).

In other embodiments, the assay comprises: immobilizing a variant of SARS-CoV-2 nucleocapsid protein, spike protein and/or spike protein RBD (receptor binding domain); adding the sample containing antibody: and detecting the amount of antibody bound to the variant, e.g., by adding another variant conjugated, directly or indirectly, to a label (e.g., metallic nanoparticle or metallic nanoshell, fluorescent label, or enzyme (e.g., horseradish peroxidase or alkaline phosphatase)) or by adding a labeled substance, such as a binding partner or a labeled antibody which specifically recognizes the sample antibody (e.g., anti-human IgG antibody, or anti-human IgM antibody).

In other embodiments, the assay comprises: immobilizing a variant of SARS-CoV-2 nucleocapsid protein, spike protein and/or spike protein RBD (receptor binding domain); adding the sample containing antibody: and detecting the amount of antibody bound to the variant, e.g., by adding a first binding partner which specifically recognizes the sample antibody (e.g., anti-human IgG antibody, or anti-human IgM antibody), and further adding a second binding partner, wherein the second binding partner is labeled and recognizes said first binding partner.

In still other embodiments, the assay comprises: reacting the variant of SARS-CoV-2 nucleocapsid protein, spike protein and/or spike protein receptor binding domain (RBD) and the sample containing antibody without any of the reactants being immobilized, and then detecting the amount of complexes of an antibody and the variant, e.g., by the variant being labeled or by adding a labeled substance, such as a labeled binding partner (e.g., streptavidin-HRP or streptavidin colloidal gold complex) or a labeled antibody which specifically recognizes the variant.

Immobilization of a variant can be either covalent or non-covalent, and the non-covalent immobilization can be non-specific (e.g., non-specific binding to a polystyrene surface in a microtiter well). Specific or semi-specific binding to a solid or semi solid carrier, support or surface, can be achieved by the variant of SARS-CoV-2 nucleocapsid protein, spike protein and/or spike protein receptor binding domain (RBD) having, associated with it, a moiety which enables its covalent or non-covalent binding to the solid or semi-solid carrier, support or surface. For example, the moiety can have affinity to a component attached to the carrier, support or surface. In this case, the moiety may be, for example, a biotin or biotinyl group or an analogue thereof bound to an amino acid group of the variant of SARS-CoV-2 nucleocapsid protein, spike protein and/or spike protein RBD (receptor binding domain), and the component is then avidin, streptavidin, neutravidin, or an analogue thereof.

Suitable carriers, supports, and surfaces include, but are not limited to, metallic nanolayers, beads (e.g., magnetic beads, colloidal particles or metallic nanoparticles or nanoshells, such as colloidal gold, or particles or nanoparticles comprising silica, latex, polystyrene, polycarbonate, or PDVF), latex of co-polymers such as styrene-divinyl benzene, hydroxylated styrene-divinyl benzene, polystyrene, carboxylated polystyrene, beads of carbon black, non-activated or polystyrene or polyvinyl chloride activated glass, epoxy activated porous magnetic glass, gelatin or polysaccharide particles or other protein particles, red blood cells, mono- or polyclonal antibody or Fab fragments of such antibody.

The protocols for immunoassays using antigens for detection of specific antibody are well known in art. For example, a conventional sandwich assay can be used, or a conventional competitive assay format can be used.

Devices for performing specific binding assays, especially immunoassays, are known and can be readily adapted for use in the present methods. Solid-phase assay devices include microtiter plates, flow-through assay devices (e.g., lateral flow immunoassay devices), dipsticks, and immunocapillary or immunochromatographic immunoassay devices.

In embodiments, the solid or semi-solid surface or carrier is the floor or wall in a microtiter well, a filter surface or membrane (e.g., a nitrocellulose membrane or a PVDF (polyvinylidene fluoride) membrane), a hollow fiber, a beaded chromatographic medium (e.g., an agarose or polyacrylamide gel), a magnetic bead, a fibrous cellulose matrix, an HPLC matrix, an FPLC matrix, a substance having molecules of such a size that the molecules with the variant of SARS-CoV-2 nucleocapsid protein, spike protein and/or spike protein receptor binding domain (RBD) bound thereto, when dissolved or dispersed in a liquid phase, can be retained by means of a filter, a substance capable of forming micelles or participating in the formation of micelles allowing a liquid phase to be changed or exchanged without entraining the micelles, a water-soluble polymer, or any other suitable carrier, support or surface.

In some embodiments, the variant of SARS-CoV-2 nucleocapsid protein, spike protein and/or spike protein receptor binding domain (RBD) is provided with a suitable label which enables detection. Conventional labels may be used which are capable, alone or in concert with other compositions or compounds, of providing a detectable signal. Suitable labels include, but are not limited to, enzymes (e.g., HRP, beta-galactosidase, or alkaline phosphatase), fluorescent labels, radioactive labels, colored latex particles, and metal-conjugated labels (e.g., metallic nanolayers, metallic nanoparticle- or metallic nanoshell-conjugated labels). Suitable metallic nanoparticle or metallic nanoshell labels include, but are not limited to, gold particles, silver particles, copper particles, platinum particles, cadmium particles, composite particles, gold hollow spheres, gold-coated silica nanoshells, and silica-coated gold shells. Metallic nanolayers suitable for detectable layers include nanolayers comprised of cadmium, zinc, mercury, and noble metals, such as gold, silver, copper, and platinum.

Suitable detection methods include, but are not limited to, detection of an agent which is tagged, directly or indirectly, with a colorimetric assay (e.g., for detection of HRP or beta galactosidase activity), visual inspection using light microscopy, immunofluorescence microscopy, including confocal microscopy, or by flow cytometry (FACS), autoradiography (e.g., for detection of a radioactively labeled agent), electron microscopy, immunostaining, subcellular fractionation, or the like. In one embodiment, a radioactive element (e.g., a radioactive amino acid) is incorporated directly into the variant. In another embodiment, a fluorescent label is associated with the variant via biotin/avidin interaction, association with a fluorescein conjugated antibody, or the like. In one embodiment, a detectable specific binding partner for the antibody is added to the mixture. For example, the binding partner can be a detectable secondary antibody or other binding agent (e.g., protein A, protein G, protein L or combinations thereof) which binds to the first antibody. This secondary antibody or other binding agent can be labeled with, for example, a radioactive, enzymatic, fluorescent, luminescent, metallic nanoparticle or metallic nanoshell (e.g. colloidal gold), or other detectable label, such as an avidin/biotin system. In another embodiment, the binding partner is a variant of SARS-CoV-2 nucleocapsid protein, spike protein and/or spike protein RBD (receptor binding domain), which can be conjugated directly or indirectly to an enzyme, such as horseradish peroxidase or alkaline phosphatase or other signaling moiety. In such embodiments, the detectable signal is produced by adding a substrate of the enzyme that produces a detectable signal, such as a chromogenic, fluorogenic, or chemiluminescent substrate.

In some embodiments, the detection procedure comprises visibly inspecting a complex comprising an antibody and a variant of SARS-CoV-2 nucleocapsid protein, spike protein and/or spike protein receptor binding domain (RBD) complex for a color change, or inspecting the complex for a physical-chemical change. Physical-chemical changes may occur with oxidation reactions or other chemical reactions. They may be detected by eye, using a spectrophotometer, or the like.

One assay format is a lateral flow immunoassay format. Antibodies to human or animal immunoglobulins, can be labeled with a signal generator or reporter (e.g., colloidal gold) that is dried and placed on a glass fiber pad (sample application pad or conjugate pad). The diagnostic a variant of SARS-CoV-2 nucleocapsid protein, spike protein and/or spike protein receptor binding domain (RBD) is immobilized on membrane, such as nitrocellulose or a PVDF (polyvinylidene fluoride) membrane. When a sample is applied to the sample application pad (or flows through the conjugate pad), it dissolves the labeled reporter, which then binds to all antibody in the sample. The resulting complexes are then transported into the next membrane (PVDF or nitrocellulose containing the variant by capillary action. If antibody against the variant are present, they bind to the variant striped on the membrane, thereby generating a signal (e.g., a band that can be seen or visualized). An additional antibody specific to the labeled antibody or a second labeled antibody can be used to produce a control signal.

An alternative format for the lateral flow immunoassay comprises the variant of SARS-CoV-2 nucleocapsid protein, spike protein and/or spike protein receptor binding domain (RBD) or compositions being conjugated to a ligand (e.g., biotin) and complexed with labeled ligand receptor (e.g., streptavidin-colloidal gold). The labeled complex can be placed on the sample application pad or conjugate pad. Anti-human IgG/IgM or anti animal IgG/IgM antibody are immobilized on a membrane, such as nitrocellulose of PVDF, at a test site. When sample is added to the sample application pad, an antibody in the sample reacts with a labeled variant such that the antibody becomes indirectly labeled. The antibody in the sample are then transported into the next membrane (PVDF or nitrocellulose containing the variant) by capillary action and bind to the immobilized anti-human IgG/IgM or anti-animal IgG/lgM antibody. If any of the sample antibody are bound to the variant, the label associated with the variant can be seen or visualized at the test site.

Another assay for the screening of blood products or other physiological or biological fluids is an enzyme linked immunosorbent assay, i.e., an ELISA. In some embodiments, in an ELISA, a variant of SARS-CoV-2 nucleocapsid protein, spike protein and/or spike protein receptor binding domain (RBD) is adsorbed to the surface of a microtiter well directly or through a capture matrix (e.g., an antibody). Residual, non-specific protein-binding sites on the surface are then blocked with an appropriate agent, such as bovine serum albumin (BSA), heat-inactivated normal goat serum (NGS), or BLOTTO (a buffered solution of nonfat dry milk which also contains a preservative, salts, and an antifoaming agent). The well is then incubated with a biological sample suspected of containing specific antibody. The sample can be applied neat, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound protein and then incubated with an optimal concentration of an appropriate anti-immunoglobulin antibody that is conjugated to an enzyme or other label by standard procedures and is dissolved in blocking buffer. The label can be chosen from a variety of enzymes, including horseradish peroxidase (HRP), beta-galactosidase, alkaline phosphatase (ALP), and glucose oxidase. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length). Conditions for performing ELISA assays are well-known in the art.

In another embodiment of an ELISA, a variant of SARS-CoV-2 nucleocapsid protein, spike protein and/or spike protein receptor binding domain (RBD) is immobilized on a surface, such as a ninety-six-well ELISA plate A sample is then added and the assay proceeds as above.

In still other embodiments, a variant of SARS-CoV-2 nucleocapsid protein, spike protein and/or spike protein receptor binding domain (RBD) is electro- or dot-blotted onto nitrocellulose paper. Subsequently, a sample, such as a biological fluid (e.g., serum or plasma) is incubated with the blotted antigen, and antibody in the biological fluid is allowed to bind to the antigen(s). The bound antibody can then be detected, e.g., by standard immunoenzymatic methods or by visualization using metallic nanoparticles or nanoshells coupled to secondary antibody or other antibody binding agents or combinations thereof.

It should be understood by one of skill in the art that any number of conventional protein assay formats, particularly immunoassay formats, may be designed to utilize the variant of SARS-CoV-2 nucleocapsid protein, spike protein and/or spike protein receptor binding domain (RBD) for the detection of SARS-CoV-2 antibody in a subject. This disclosure is thus not limited by the selection of the particular assay format, and is believed to encompass assay formats that are known to those of skill in the art. To date most serology has been performed using singleplex ELISA, complement fixation or neutralization assays. More recently, Luminex-based systems have been employed that can address up to 100 antigenic targets simultaneously (i.e., 100 individual pathogens, 100 individual antigenic targets for one pathogen, or some variation thereof) (Anderson et al. J. Immunol. Methods 2011; 366:79-88). Additionally, arrays are established that comprise spotted recombinant proteins expressed in vitro in E. coli, S. cerevisiae, baculoviruses, or cell-free, coupled transcription-translation systems (Vigil et al. Future Microbiol. 2010:5:241-51).

One goal of the present disclosure is to automate the process of SARS-CoV-2 antibody detection and make it inexpensive, quick and accurate as well as detect exposure per se rather than to rigorously characterize humoral responses to specific pathogens.

One assay that meets these requirements is a programmable array. One method to create and validate a programmable array that can measure the humoral immune response to SARS-CoV-2, thus enabling detection of exposure to SARS-CoV-2 (or its gene products in vaccines), comprises the following steps: 1) select a variant of SARS-CoV-2 nucleocapsid protein, spike protein and/or spike protein receptor binding domain (RBD) using bioinformatic methods; 2) test the variant printed on arrays for sensitivity and specificity using sera from humans and other animals who have been exposed to antigens of SARS-CoV-2; 3) examine the performance of algorithms typically employed for epitope prediction; 4) use assay results to develop smaller and less comprehensive variant libraries that can be deployed in smaller and more facile platforms, 5) optimize and validate assay protocols; and 6) develop software to automate assay analysis.

The presence of an elevated amount of a complex comprising an antibody and a variant of SARS-CoV-2 nucleocapsid protein, spike protein and/or spike protein receptor binding domain (RBD) complex indicates that the subject was exposed to and infected by the SARS-CoV-2 virus. A variant of SARS-CoV-2 nucleocapsid protein, spike protein and/or spike protein RBD (receptor binding domain), including a modified form thereof, which “binds specifically” to an antibody against a SARS-CoV-2 antigen interacts with the antibody, or forms or undergoes a physical association with it, in an amount and for a sufficient time to allow detection of the antibody.

Conditions for reacting a variant protein and antibody so that they react specifically are well known to those of skill in the art. See, e.g., Current Protocols in Immunology (Coligan et al., editors, John Wiley & Sons, Inc).

Once the variant of SARS-CoV-2 nucleocapsid protein, spike protein and/or spike protein receptor binding domain (RBD) and sample antibody are permitted to react in a suitable medium, an assay is performed to determine the presence or absence of the reaction. Any of the assays discussed herein can be used.

The methods and systems of the present disclosure may be used to detect exposure to antigens of SARS-CoV-2 in research and clinical settings.

One sample for use in the methods is a biological sample. A biological sample may be obtained from a tissue of a subject or bodily fluid from a subject including but not limited to nasopharyngeal aspirate, blood, cerebrospinal fluid, saliva, serum, plasma, urine, sputum, bronchial lavage, pericardial fluid, or peritoneal fluid, or a solid such as feces. The preferred biological sample is serum, plasma and urine. The subject may be any animal, particularly a vertebrate and more particularly a mammal, including, without limitation, a cow, dog, human, monkey, mouse, pig, or rat. In one embodiment, the subject is a human.

A sample may also be a research, clinical, or environmental sample, such as cells, cell culture, cell culture medium, and compositions for use as, or the development of pharmaceutical and therapeutic agents.

Additional applications include, without limitation, detection of the screening of blood products (e.g., screening blood products for infectious agents), biodefense, food safety, environmental contamination, forensics, and genetic-comparability studies. The present disclosure also provides methods and systems for detecting viral antibody in cells, cell culture, cell culture medium and other compositions used for the development of pharmaceutical and therapeutic agents.

The subject may have been exposed to antigens of SARS-CoV-2, suspected of having exposure to antigens of SARS-CoV-2 or believed not to have had exposure to antigens of SARS-CoV-2. In one embodiment, the subject is a female, and in a further embodiment the female subject may be pregnant or attempting to become pregnant. In one embodiment, the subject may have been found to be seropositive by SARS-CoV-2 ELISA.

In one embodiment, the subject may be a test subject, which has been administered a SARS-CoV-2 vaccine or immunomodulatory agent.

The systems and methods described herein support the detection and measure of a humoral immune response to SARS-CoV-2.

The method may optionally include a step for washing any unbound sample.

Any method of detection discussed herein or known in the art can be used for visualizing and/or quantifying the bound antibody.

The disclosure also includes reagents and kits for practicing the methods. These reagents and kits may vary.

In some embodiments, the variant of SARS-CoV-2 nucleocapsid protein, spike protein and/or spike protein receptor binding domain (RBD) are attached to or immobilized on a solid support through a metallic nanolayer (e.g., cadmium, zinc, mercury, gold, silver, copper, or platinum nanolayer). In certain embodiments, the solid support is a bead (e.g., a colloidal particle or a metallic nanoparticle or nanoshell), a flow path in a lateral flow immunoassay device, a flow path in an analytical or centrifugal rotor, a tube or a well (e.g., in a plate), or a sensor (e.g., an electrochemical, optical, or opto-electronic sensor).

Reagents for particular types of assays can also be provided in kits. Thus, the kits can include a population of beads (e.g., suitable for an agglutination assay or a lateral flow assay), or a plate (e.g., a plate suitable for an ELISA assay). In other embodiments, the kits comprise a device, such as a lateral flow immunoassay device, an analytical or centrifugal rotor, a Western blot, a dot blot, a slot blot, or an electrochemical, optical, or opto-electronic sensor.

In one particular embodiment, the kit would comprise a variant of SARS-CoV-2 nucleocapsid protein, spike protein and/or spike protein receptor binding domain (RBD) microarrays comprising a variant of SARS-CoV-2 nucleocapsid protein, a variant of the spike protein and/or a variant of the spike protein receptor binding domain (RBD) or a collection or set of a variant of SARS-CoV-2 nucleocapsid protein, spike protein and/or spike protein RBD (receptor binding domain), which are reactive with, and specific for SARS-CoV-2 antibody, comprising: the variant of SARS-CoV-2 nucleocapsid protein, spike protein and/or spike protein receptor binding domain (RBD) comprising the amino acid sequence DITWEKDAEXTGNSPRLDVA, wherein X is V or I (SEQ ID NO: 4792); or a variant of SARS-CoV-2 nucleocapsid protein, spike protein and/or spike protein receptor binding domain (RBD) chosen from the group consisting of the variant of SARS-CoV-2 nucleocapsid protein, spike protein and/or spike protein receptor binding domain (RBD) listed in SEQ ID NOs: 2-13; or a collection or set of a variant of SARS-CoV-2 nucleocapsid protein, spike protein and/or spike protein receptor binding domain (RBD) comprising amino acid sequences shifted one residue across the variant of SARS-CoV-2 nucleocapsid protein, spike protein and/or spike protein receptor binding domain (RBD) comprising the amino acid sequence DITWEKDAEXTGNSPRLDVA, wherein X is V or I (SEQ ID NO: 4792) (i.e., SEQ ID NOs: 14-22); or a collection or set of a variant of SARS-CoV-2 nucleocapsid protein, spike protein and/or spike protein receptor binding domain (RBD) comprising amino acid sequences shifted one residue across at least one a variant of SARS-CoV-2 nucleocapsid protein, spike protein and/or spike protein receptor binding domain (RBD) chosen from the group consisting of a variant of SARS-CoV-2 nucleocapsid protein, spike protein and/or spike protein receptor binding domain (RBD) listed in SEQ ID NOs: 2-13; or combinations thereof.

In addition, the kits can include various diluents and buffers, labeled conjugates or other agents for the detection of specifically bound antigens or antibody (e.g. labeling reagents), and other signal-generating reagents, such as enzyme substrates, cofactors and chromogens. In some embodiments, the kit comprises an anti-human IgG/lgM antibody conjugated to a detectable label (e.g., a metallic nanoparticle, metallic nanoshell, metallic nanolayer, fluorophore, colored latex particle, or enzyme) as a labeling reagent. In other embodiments, the kit comprises protein A, protein G, protein A/G fusion proteins, protein L, or combinations thereof conjugated to a detectable label (e.g., a metallic nanoparticle, metallic nanoshell, metallic nanolayer, fluorophore, colored latex particle, or enzyme) as a labeling reagent. In still other embodiments, the labeling reagents of the kit are a second collection or set of a variant of SARS-CoV-2 nucleocapsid protein, spike protein and/or spike protein receptor binding domain (RBD) conjugated to a detectable label (e.g., a metallic nanoparticle, metallic nanoshell, metallic nanolayer, fluorophore, colored latex particle, or enzyme). The second collection or set of a variant of SARS-CoV-2 nucleocapsid protein, spike protein and/or spike protein receptor binding domain (RBD) can be the same as or different than the collection or set of a variant of SARS-CoV-2 nucleocapsid protein, spike protein and/or spike protein RBD (receptor binding domain), which may optionally be attached to or immobilized upon a solid support.

Other components of a kit can easily be determined by one of skill in the art. Such components may include coating reagents, polyclonal or monoclonal capture antibody specific for a variant of SARS-CoV-2 nucleocapsid protein, spike protein and/or spike protein RBD (receptor binding domain), or a cocktail of two or more of the antibody, purified or semi-purified extracts of these antigens as standards, monoclonal antibody detector antibody, an anti-mouse, anti-dog, anti-cat, anti-chicken, or anti-human antibody conjugated to a detectable label, indicator charts for colorimetric comparisons, disposable gloves, decontamination instructions, applicator sticks or containers, a sample preparatory cup and buffers or other reagents appropriate for constituting a reaction medium allowing the formation of a variant of SARS-CoV-2 nucleocapsid protein, spike protein and/or spike protein RBD (receptor binding domain)-antibody complex.

Such kits provide a convenient, efficient way for a clinical laboratory to diagnose infection by SARS-CoV-2 virus. Thus, in certain embodiments, the kits further comprise instructions.

### Methods of Treatment

Provided herein are methods of treating and/or preventing a SARS-CoV-2 infection comprising administering the composition disclosed herein to a subject (e.g., administering vaccine compositions comprised protein variants and/or nucleic acids disclosed herein). In some embodiments, the composition comprises multiple variants provided herein (e.g., in some embodiments, the composition comprises a nucleocapsid variant, a spike protein variant and/or a spike protein RBD variant). The methods may comprise administering one dose of the composition or multiple doses of the composition.

Also disclosed herein are methods for inducing an immune response to a SARS-CoV-2 using the disclosed variants. Generally, the vaccine and/or composition may be administered subcutaneously, intradermally, submucosally, intranasally, or intramuscularly in an effective amount to prevent infection from the SARS-CoV-2 and/or treat an infection from the SARS-CoV-2. An effective amount to prevent infection is an amount of variants disclosed herein that will induce immunity in the immunized animals against challenge by a virulent virus such that infection is prevented or the severity is reduced. Immunity is defined herein as the induction of a significant higher level of protection in a subject after immunization compared to an unimmunized group. An effective amount to treat an infection is an amount of immunizing protein that induces an appropriate immune response against SARS-CoV-2 such that severity of the infection is reduced.

Protective immune responses can include humoral immune responses and cellular immune responses. Protection against SARS-CoV-2 is may be conferred through serum antibodies (humoral immune response) directed to the surface proteins. Cellular immune responses are useful in protection against SARS-CoV-2 virus infection with CD4+ and CD8+ T cell responses being particularly important. CD8+ immunity is of particular importance in killing virally infected cells.

Additionally, the disclosed variants and/or compositions can be administered using immunization schemes known by persons of ordinary skill in the art to induce protective immune responses. These include a single immunization or multiple immunizations in a prime-boost strategy. A boosting immunization can be administered at a time after the initial, prime, immunization that is days, weeks, months, or even years after the prime immunization. In certain embodiments, a boost immunization is administered 2 weeks, 1 month, 2, months, 3 months, 4 months, 5 months, or 6 months or more after the initial prime immunization. Additional multiple boost immunizations can be administered such as weekly, every other week, monthly, every other month, every third month, or more. In other embodiments, the boost immunization is administered every 3 weeks, every 4 weeks, every 5 weeks, every 6 weeks, every 7 weeks, every 8 weeks, every 9 weeks, every 10 weeks, every 11 w weeks, or every 12 weeks. In certain embodiments, boosting immunizations can continue until a protective anti-SARS-CoV-2 antibody titer is seen in the subject's serum. In certain embodiments, a subject is given one boost immunization, two boost immunizations, three boost immunizations, or four or more boost immunizations, as needed to obtain a protective antibody titer. In other embodiments, the adjuvant in the initial prime immunization and the adjuvant in the boost immunizations are different.

### Methods of Manufacture

Provided herein are methods of making a protein variant disclosed herein. The methods may comprise transfecting a cell with a nucleic acid disclosed herein and expanding the cells under conditions that allow cell propagation. The methods may further comprise isolating the protein variants from the propagated cells.

The level of protein variant of any of the variants described herein can be increased in a cell, e.g., by introducing into the cell a nucleic acid encoding the variant protein operably linked to a transcriptional regulatory sequence directing the expression of the protein in the cell. Methods for expressing nucleic acids in cells and appropriate transcriptional regulatory elements for doing so are well known in the art. Alternatively, any of the variant proteins described herein can be introduced into a cell, usually in the presence of a vector facilitating the entry of the protein into the cells, e.g., liposomes. Proteins can also be linked to transcytosis peptides for that purpose.

Host Cells

The protein variants may be expressed in any suitable expression system, including any suitable host cell. Suitable host cells include, but are not limited to: yeast cells, bacterial cells, algal cells, plant cells, fungal cells, insect cells, and animal cells, including mammalian cells. In one illustrative embodiment, suitable host cells include E. coli (e.g., Shuflle™ competent E. coli available from New England BioLabs in Ipswich, Mass.).

Other suitable host cells of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments the preferred host cell of the present disclosure is C. glutamicum.

Suitable host cells of the genus Corynebacterium, in particular of the species Corynebacterium glutamicum, are in particular the known wild-type strains: Corynebacterium glutamicum ATCC 13032, Corynebacterium acetoglutamicum ATCC15806, Corynebacterium aceloacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricantum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccharomyces carlsbergensis, Saccharomyces diastaticus, Saccharomyces norbensis, Saccharomyccs kluyveri, Schizosaccharomyces pombe, Komagataella phaffii, formerly known as Pichia pastoris, Pichia finlandica, Pichia trhalophila, Pichia kodamac, Pichia membranaefaciens, Pichia opuntiae, Pichia thermotolerans. Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyriwbrio, Buchnera, Campestris, Camplynbacter, Closiridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacteriuim, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosvnechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

The genetically modified organisms provided herein include microorganisms and plants. In some embodiments, the genetically modified organisms are microorganisms. In some embodiments, the microorganisms are selected from yeast, bacteria, microalgae, insect cells, and plant cells. In some embodiments, the organisms are selected from yeast and bacteria.

### EXEMPLIFICATION

### Design and Screen of ECT Spike Variants

Coronaviruses (e.g., beta coronaviruses SARS-CoV, MERS-CoV, OC43, and HKU1; and alpha coronaviruses 229E and NL63) express a large repertoire of native antigens. Of prime focus is the spike protein, which interacts directly with the human ACE2 receptor and at present is the key antigen utilized in SARS-CoV 2 vaccines. One of the greatest challenges for spike-based vaccine development is stabilization of the pre-fusion conformation, which is the key structural conformation to maintain such that a robust and neutralizing antibody repertoire is elicited by the human body. The unstable immunogenic pre-fusion conformation of the SARS-CoV 2 spike protein was stabilized for cryo-electron microscopy (cryo-EM) by inclusion of two proline mutations in the S2 fusion domain. Despite these modifications, expression titers were low, suggesting room for further improvement in stability, which correlates with expression level. Applicant has created variants with additional mutations that can be introduced to improve stability and has identified unique, novel mutations to the spike protein which confer enhanced biophysical properties, chiefly stabilization of the pre-fusion conformation, yet retain their biological function of ACE2 and neutralizing antibody binding.

Applicant used “template” (Antigen ID 4155326), and added additional mutations to the ECT spike sequence. All variants described in this Example contain the mutations R682S, R683G, R685G, K986P, and V987P, in addition to the novel mutations designed and tested.

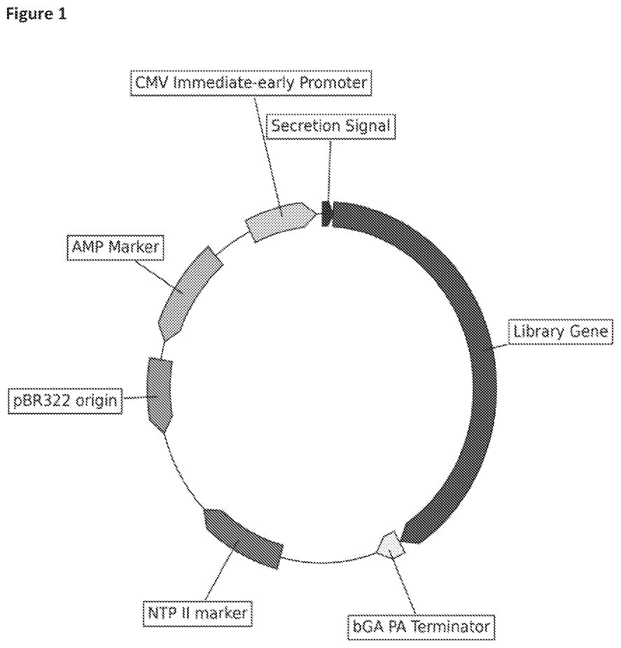

In an effort to engineer SARS-CoV 2 ECT spike variants for enhanced stability, producibility, and maintained or enhanced binding properties to known neutralizing antibodies and to the ACE2 human receptor, a library of approximately 600 unique variants of the ECT spike template protein were designed. This was done using a combination of structure and sequence based design approaches. The resultant designs were placed into a mammalian protein expression construct (FIG. 1) and ordered for DNA synthesis. Transfection grade DNA was then prepared from the synthesized plasmids, and from the initial set of 600 variant designs a total of 571 were successfully synthesized and had transfection-grade DNA prepared. The resultant set of plasmids were transiently transfected into HEK293 cells, and after culturing the supernatants containing secreted proteins were harvested by centrifugation and used directly and immediately in subsequent assays.

Supernatants containing variants of SARS-CoV 2 spike antigen were assessed for: (1) protein expression yield; (2) binding to recombinant human ACE2; (3) binding to pooled sera from humans isolated both pre-pandemic as well as from SARS-CoV 2 positive patients. Protein expression yields for variants ranged from 0% to 500% compared to the template, with approximately one sixth of the library (one hundred variants) demonstrating significantly improved expression yield. A collection of 17 hits from this collection, selected based on expression improvements and functional maintenance (sera and ACE2 binding), were advanced to further characterization in follow-up experiments.

In the follow up experiment, replicate 40 mL mammalian transient transfection expression cultures were prepared in culture flasks and the expressed proteins were purified from supernatants via Ni-NTA affinity chromatography using an FPLC. The eluted proteins were buffer exchanged, quantified, and aliquoted for storage at −80° C. Preparations were thawed on ice.

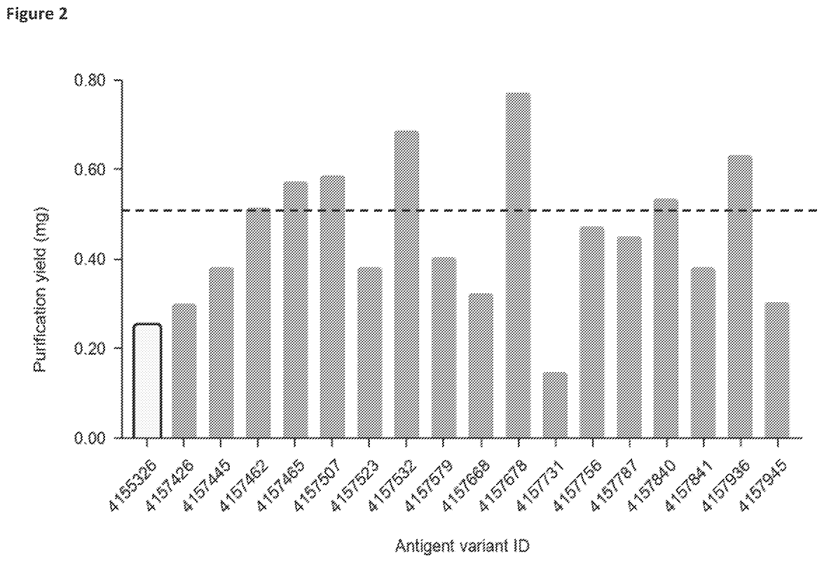

Quantification of the purified protein preps revealed that all but one (4157731) of the variants yielded more protein than the template (Table 8 and FIG. 2), indicating that these variants have enhanced biophysical properties which correlate with yield such as stability and solubility. Seven of the variants (4157678, 4157532, 4157936, 4157507, 4157465, 4157840, 4157462) demonstrated >2-fold improved protein yield compared to the template, and the best yielding variant, 4157678, demonstrated a 3.1-fold improvement in yield. This variant contained the mutation T768E relative to the template sequence. SEQ ID NOs for each variant tested can be found in Table 8.

TABLE 8

| | | Concentration | Purification yield |
|---|---|---|---|
| Variant ID | SEQ ID NO: | (mg/ml) | (mg) |
| 4155326 | Control | 0.066 | 0.25 |
| 4157426 | 1795 | 0.088 | 0.30 |
| 4157445 | 1962 | 0.091 | 0.38 |
| 4157462 | 1804 | 0.123 | 0.52 |
| 4157465 | 1805 | 0.084 | 0.57 |
| 4157507 | 2097 | 0.125 | 0.59 |
| 4157523 | 1824 | 0.083 | 0.38 |
| 4157532 | 2103 | 0.156 | 0.69 |
| 4157579 | 1996 | 0.112 | 0.40 |
| 4157668 | 2135 | 0.077 | 0.32 |
| 4157678 | 2343 | 0.168 | 0.77 |
| 4157731 | 2285 | 0.045 | 0.15 |
| 4157756 | 2156 | 0.115 | 0.47 |
| 4157787 | 2016 | 0.100 | 0.45 |
| 4157840 | 1812 | 0.127 | 0.53 |
| 4157841 | 1916 | 0.109 | 0.38 |
| 4157936 | 1955 | 0.154 | 0.63 |
| 4157945 | 2359 | 0.082 | 0.30 |

*Total mass (mg) and concentration (mg/ml) of the purified yield from 17 antigen variants. Variant ID 4155326 is the template for all the variants.

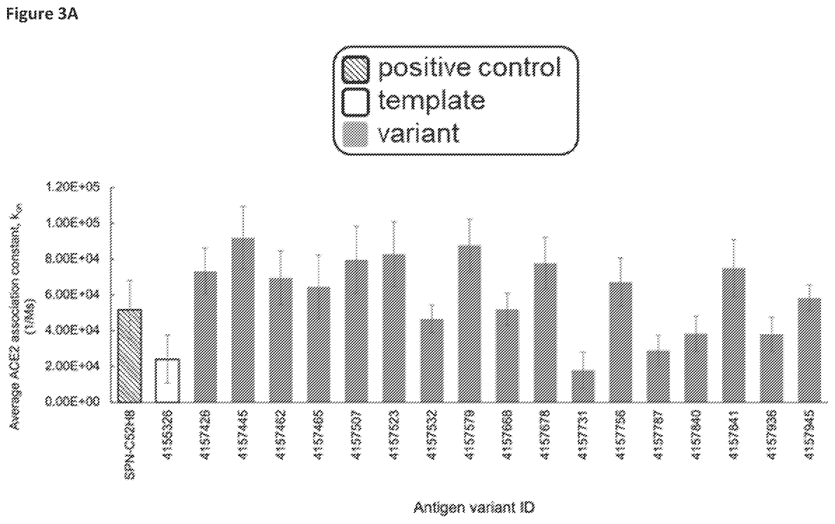

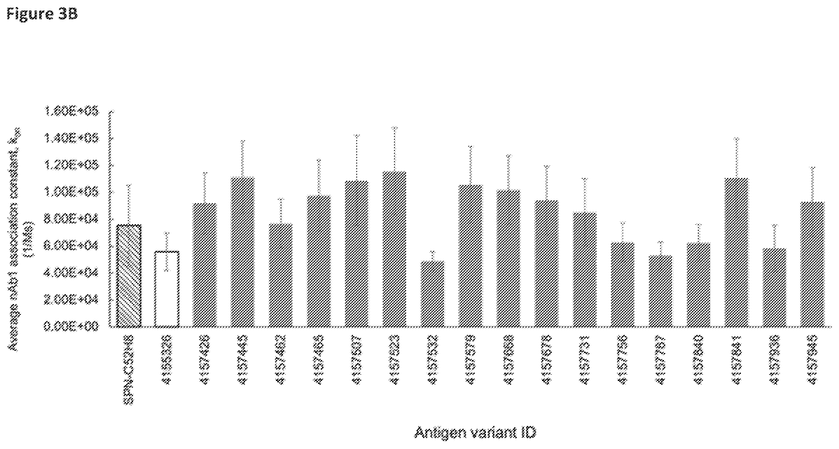

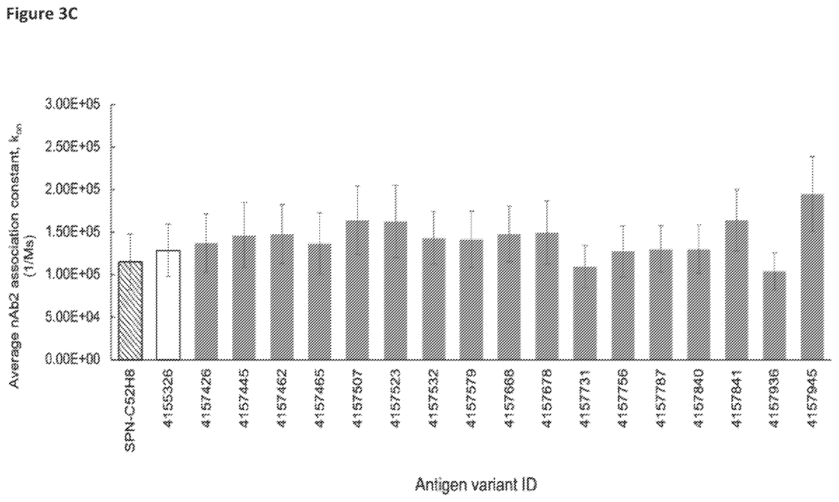

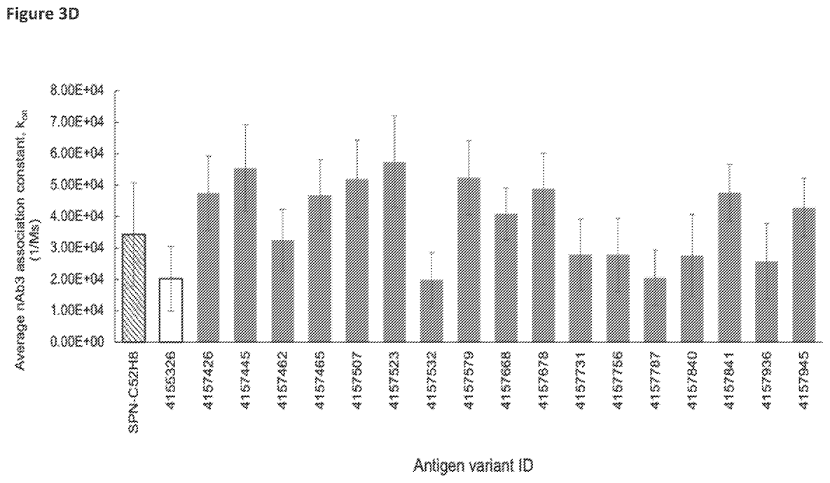

To assess the binding characteristics of the template and variants, Octet binding characterization experiments were performed using ACE2 and three different commercially available neutralizing antibodies (nAbs), deemed nAb1-3. Given the sensitivity limitations of the Octet instrument, the association constant, kₒₙ, was utilized as the primary metric for binding characterization, as it is generally the most reliable and robust metric derived from an Octet binding curve. A commercially available ECT spike protein (SPN-C52H8 from Acro Biosystems) was used as a positive control. All 17 variants demonstrated indistinguishable or improved kₒₙ as compared to the template antigen for all binding targets tested (Table 9 and FIG. 3). For FIG. 3A-D, Antigen ID 4155326, representing the template (starting) antigen sequence, was used as a positive control and to establish a baseline for comparison of binding constants. The data represent the average of four technical replicates±one standard deviation of the mean. Antigen IDs and their corresponding binding constants from these graphs are shown in Table 9.

TABLE 9

| | ACE2 | ACE2 | nAb1 | nAb1 | nAb2 | nAb2 | nAb3 | nAb3 |
|---|---|---|---|---|---|---|---|---|
| | k_on | k_on | k_on | k_on | k_on | k_on | k_on | k_on |
| Variant | Average | Standard | Average | Standard | Average | Standard | Average | Standard |
| ID | (1/Ms) | Deviation | (1/Ms) | Deviation | (1/Ms) | Deviation | (1/Ms) | Deviation |
| SPN- | 5.17E+04 | 1.63E+04 | 7.54E+04 | 3.02E+04 | 1.15E+05 | 3.28E+04 | 3.43E+04 | 1.64E+04 |
| C52H8 | | | | | | | | |
| 4155326 | 2.40E+04 | 1.35E+04 | 5.60E+04 | 1.39E+04 | 1.29E+05 | 3.08E+04 | 2.02E+04 | 1.03E+04 |
| 4157426 | 7.33E+04 | 1.30E+04 | 9.19E+04 | 2.25E+04 | 1.37E+05 | 3.45E+04 | 4.74E+04 | 1.18E+04 |
| 4157445 | 9.19E+04 | 1.75E+04 | 1.1IE+05 | 2.71E+04 | 1.46E+05 | 3.84E+04 | 5.54E+04 | 1.38E+04 |
| 4157462 | 6.96E+04 | 1.51E+04 | 7.68E+04 | 1.81E+04 | 1.48E+05 | 3.49E+04 | 3.25E+04 | 9.85E+03 |
| 4157465 | 6.44E+04 | 1.77E+04 | 9.78E+04 | 2.64E+04 | 1.37E+05 | 3.59E+04 | 4.68E+04 | 1.14E+04 |
| 4157507 | 7.97E+04 | 1.88E+04 | 1.09E+05 | 3.35E+04 | 1.64E+05 | 4.02E+04 | 5.19E+04 | 1.23E+04 |
| 4157523 | 8.27E+04 | 1.82E+04 | 1.15E+05 | 3.23E+04 | 1.63E+05 | 4.26E+04 | 5.75E+04 | 1.45E+04 |
| 4157532 | 4.66E+04 | 7.73E+03 | 4.87E+04 | 7.14E+03 | 1.43E+05 | 3.11E+04 | 1.98E+04 | 8.68E+03 |
| 4157579 | 8.76E+04 | 1.46E+04 | 1.06E+05 | 2.84E+04 | 1.41E+05 | 3.32E+04 | 5.23E+04 | 1.17E+04 |
| 4157668 | 5.18E+04 | 9.03E+03 | 1.02E+05 | 2.57E+04 | 1.48E+05 | 3.25E+04 | 4.08E+04 | 8.25E+03 |
| 4157678 | 7.78E+04 | 1.44E+04 | 9.39E+04 | 2.54E+04 | 1.50E+05 | 3.71E+04 | 4.88E+04 | 1.13E+04 |
| 4157731 | 1.79E+04 | 1.01E+04 | 8.50E+04 | 2.51E+04 | 1.10E+05 | 2.47E+04 | 2.79E+04 | 1.13E+04 |
| 4157756 | 6.72E+04 | 1.34E+04 | 6.29E+04 | 1.44E+04 | 1.27E+05 | 3.00E+04 | 2.78E+04 | 1.18E+04 |
| 4157787 | 2.89E+04 | 8.49E+03 | 5.30E+04 | 1.02E+04 | 1.30E+05 | 2.75E+04 | 2.06E+04 | 8.85E+03 |
| 4157840 | 3.84E+04 | 9.86E+03 | 6.23E+04 | 1.37E+04 | 1.30E+05 | 2.87E+04 | 2.77E+04 | 1.31E+04 |
| 4157841 | 7.48E+04 | 1.61E+04 | 1.11E+05 | 2.91E+04 | 1.64E+05 | 3.60E+04 | 4.75E+04 | 9.06E+03 |
| 4157936 | 3.80E+04 | 9.46E+03 | 5.84E+04 | 1.72E+04 | 1.04E+05 | 2.19E+04 | 2.59E+04 | 1.20E+04 |
| 4157945 | 5.83E+04 | 7.22E+03 | 9.29E+04 | 2.57E+04 | 1.95E+05 | 4.42E+04 | 4.29E+04 | 9.30E+03 |

* Binding association rate against ACE2 protein and three different neutralizing antibodies for the 17 variants, 4155326 is the template for all variants. SPN-C52H8 is a commercial reagent purchased from Acro Biosystems that was used as a positive control.

The best variant for ACE2 kₒₙ improvement was 4157445, demonstrating a 3.8-fold improvement. This variant contains the mutation S50M relative to the template. For nAb binding, most variants were indistinguishable from the template or moderately improved. Variant 4157445 had a 2.7-fold improvement in kₒₙ for nAb3. Variant 4157945 containing mutation V622D demonstrated a moderately improved kₒₙ for all three nAbs. Taken together, these results indicate that in addition to the improved variants having superior protein expression and purification yield compared with the template antigen, they also maintained or demonstrated improved binding characteristics to ACE2 and each of the nAbs tested.

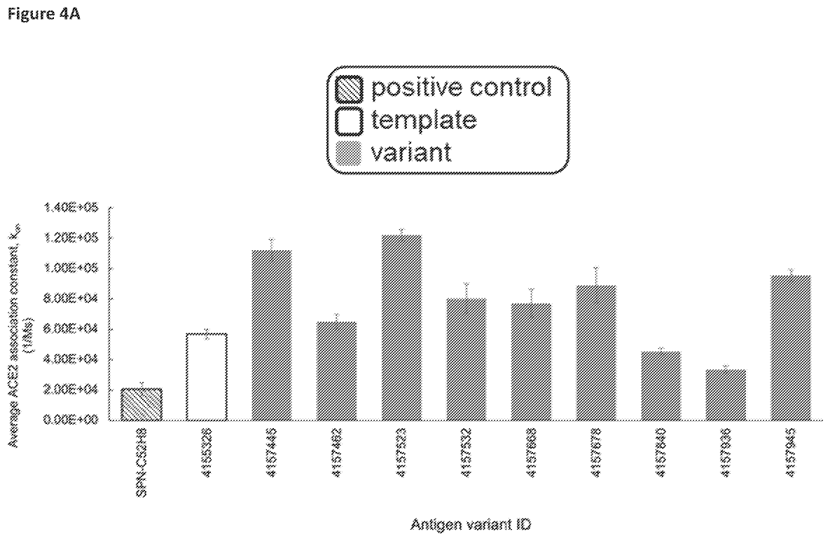

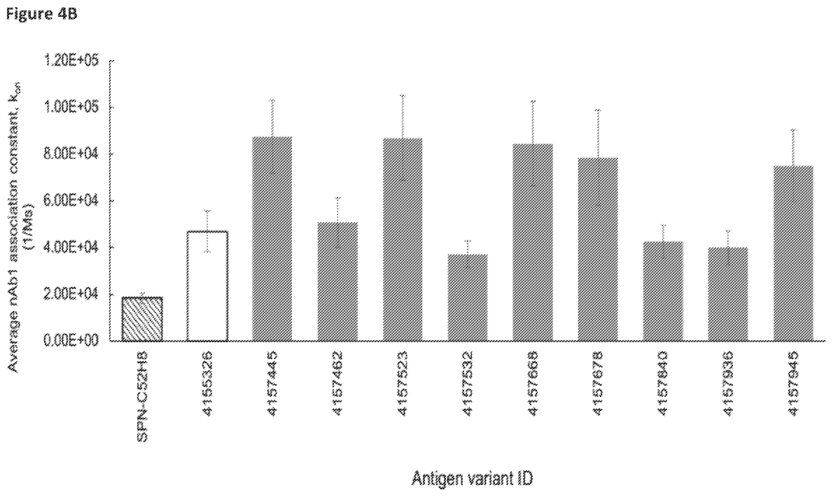

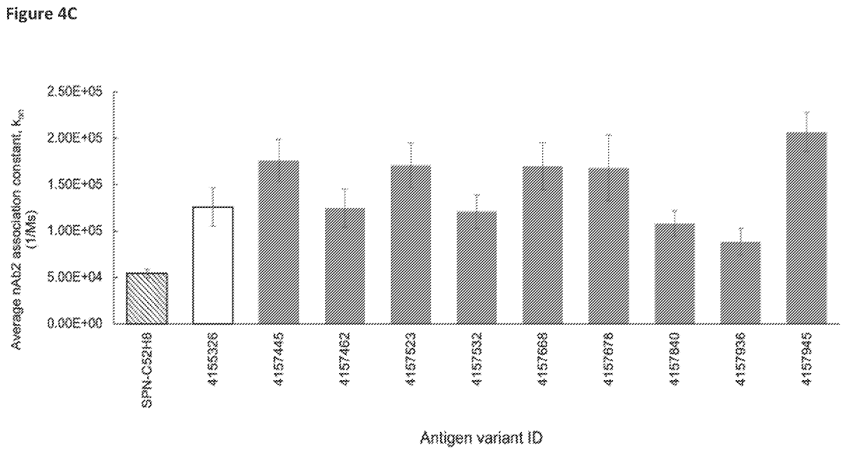

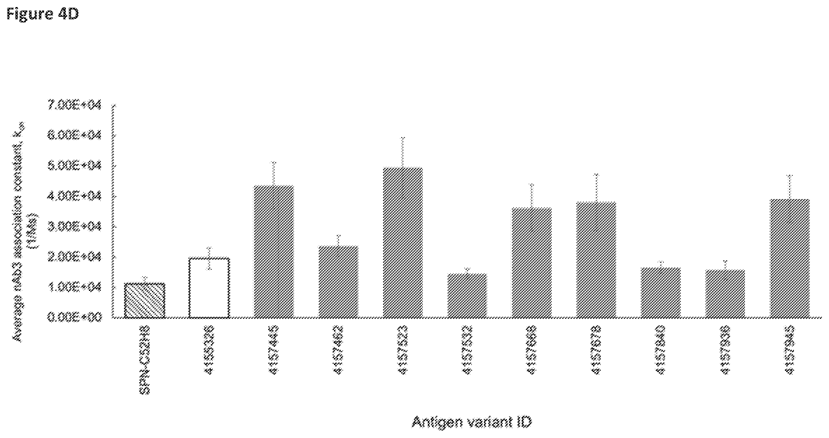

To confirm our findings, a repeat experiment of the binding characterization of the top 9 best variants was executed (Table 10 and FIG. 4). For this set of experiments, Applicant used a more sensitive Octet binding assay to enable higher resolution data collection. For FIG. 4A-4D, Antigen ID 4155326, representing the template (starting) antigen sequence, was used as a positive control and to establish a baseline for comparison of binding constants. The data represent the average of three technical replicates f one standard deviation of the mean. Antigen IDs and their corresponding binding constants from these graphs are shown in Table 10.

TABLE 10

| | ACE2 | ACE2 | nAb1 | nAb1 | nAb2 | nAb2 | nAb3 | nAb3 |
|---|---|---|---|---|---|---|---|---|
| | k_on | k_on | k_on | k_on | k_on | k_on | k_on | k_on |
| Variant | Average | Standard | Average | Standard | Average | Standard | Average | Standard |
| ID | (1/Ms) | Deviation | (1/Ms) | Deviation | (1/Ms) | Deviation | (1/Ms) | Deviation |
| SPN- | 2.05E+04 | 4.36E+03 | 1.84E+04 | 2.19E+03 | 5.43E+04 | 4.51E+03 | 1.13E+04 | 1.99E+03 |
| C52H8 | | | | | | | | |
| 4155326 | 5.68E+04 | 3.19E+03 | 4.69E+04 | 8.76E+03 | 1.26E+05 | 2.07E+04 | 1.96E+04 | 3.48E+03 |
| 4157445 | 1.12E+05 | 7.17E+03 | 8.74E+04 | 1.56E+04 | 1.76E+05 | 2.28E+04 | 4.36E+04 | 7.63E+03 |
| 4157462 | 6.50E+04 | 4.83E+03 | 5.07E+04 | 1.05E+04 | 1.25E+05 | 2.05E+04 | 2.37E+04 | 3.45E+03 |
| 4157523 | 1.22E+05 | 3.94E+03 | 8.69E+04 | 1.80E+04 | 1.71E+05 | 2.42E+04 | 4.95E+04 | 9.88E+03 |
| 4157532 | 8.02E+04 | 9.72E+03 | 3.72E+04 | 5.67E+03 | 1.21E+05 | 1.81E+04 | 1.46E+04 | 1.61E+03 |
| 4157668 | 7.72E+04 | 9.30E+03 | 8.45E+04 | 1.82B+04 | 1.70E+05 | 2.54E+04 | 3.63E+04 | 7.68E+03 |
| 4157678 | 8.90E+04 | 1.16E+04 | 7.85E+04 | 2.02E+04 | 1.68E+05 | 3.52E+04 | 3.81E+04 | 9.23E+03 |
| 4157840 | 4.54E+04 | 1.98E+03 | 4.26E+04 | 6.91E+03 | 1.08E+05 | 1.38E+04 | 1.66E+04 | 1.80E+03 |
| 4157936 | 3.36E+04 | 2.34E+03 | 3.99E+04 | 7.38E+03 | 8.87E+04 | 1.43E+04 | 1.58E+04 | 3.05E+03 |
| 4157945 | 9.54E+04 | 3.85E+03 | 7.50E+04 | 1.53E+04 | 2.07E+05 | 2.09E+04 | 3.92E+04 | 7.67E+03 |

* Binding association rate against ACE2 protein and three different neutralizing antibodies for the top nine variants from Table 9, 4155326 is the template for all variants. SPN-C52H8 is a commercial reagent purchased from Acro Biosystems that was used as a positive control.

The enhanced kₒₙ binding properties of U.S. Pat. Nos. 4,157,445 and 4,157,945 observed in the first characterization were replicated in this experiment. Additionally, variant 4157523, containing the mutation L981Y, demonstrated enhanced binding to ACE2 and each of the nAbs. This mutation is interesting as it is close in sequence space to the double proline mutations (K986P V987P) contained in the template sequence, potentially offering a positive epistatic or additive effect on the stabilization of this region of the protein. Variants 4157668 and 4157678 demonstrated enhanced kₒₙ for ACE2, nAb, and nAb3. Variant 4157678, containing mutation T768E, was also the highest yielding variant from protein expression and purification. This indicates that this mutation potentially has enhanced stabilization of the pre-fusion conformation, enabling both high-yield heterologous expression as well as moderately enhanced binding properties to cognate protein binding targets.

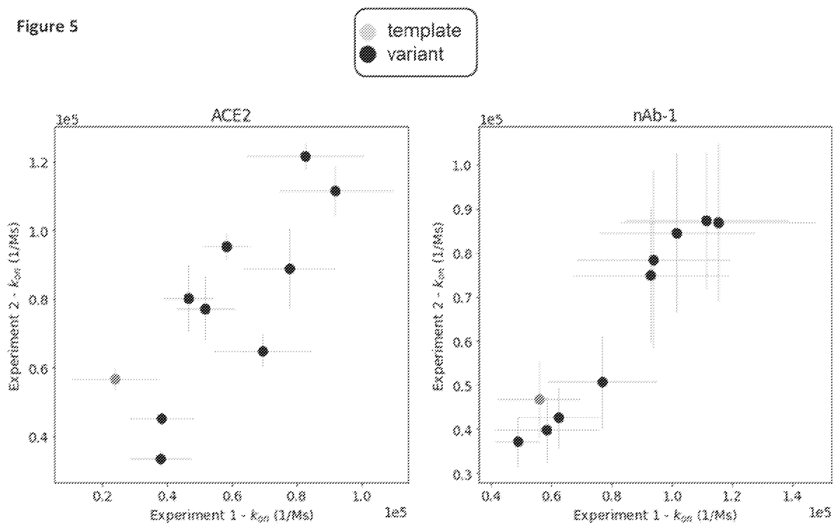

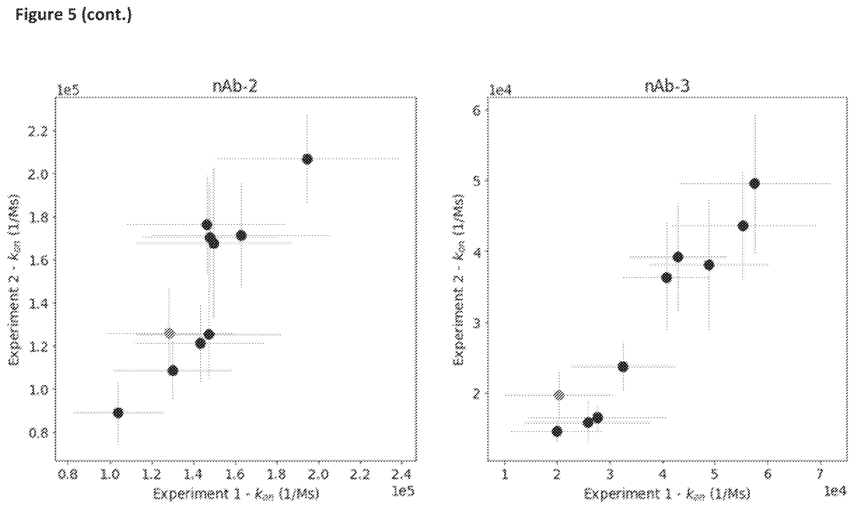

To ensure that the results of this repeat experiment agreed with the first experiment, a correlation analysis of the measured kₒₙ parameters for each of the 9 variants tested in the two experiments was performed (FIG. 5). It was found that the kₒₙ measurements for each of the four binding partners were in strong agreement between the two experiments. with Pearson's correlation coefficients equal to 0.653, 0.949, 0.769. and 0.929 for ACE2, nAb1, nAb2, and nAb3, respectively (FIG. 5).

### Materials and Methods

Mammalian Cell Growth. Transfection, and Harvest of supernatant

Expi293′M Expression Medium (Gibco) was used for all cultures, and 37° C., 8% CO₂, 120 rpm shaking was used for all incubations. On the day prior to transfection, Expi293F cell subcultures were prepared at 3×10⁶ live cells/mL. On the day of transfection, the pre-transfection cells were counted and diluted to 3×10⁶ live cells/mL with pre-warmed media, and the media-diluted cell mixture was aliquoted in 40 mL aliquots into 250 mL plain-bottom flasks and returned to the incubator. Maxi-scale transfection-grade DNA preparations for the 17 variants and the template sequence were ordered from and prepared by Genewiz. Transfection reaction and DNA were prepared in duplicate volumes in 15 mL Falcon tubes as follows. The appropriate volume of DNA to supply 80 μg of DNA was added to the tube and then the appropriate volume of Opti-MEM solution (Gibco) was added to bring the final volume to 4.8 mL and mixed by pipetting. ExpiFectamine 293 reagent (Gibco) was mixed by inversion 5 times and 256 μL of reagent was added to a second 15 mL Falcon tube then diluted with 4.8 mL Opti-MEM and incubated at room temperature for 5 minutes. 4.8 mL of the diluted ExpiFectamine solution was added to each diluted DNA preparation and swirled to mix. The mixture was incubated at room temperature for 15 minutes. 4.8 mL of the DNA transfection mixture was added to two replicate 40 mL cultures of the prepared Expi293F cells in 250 mL plain-bottom flasks. After 18-22 hours, 240 μL of Enhancer I and 2.42 mL of Enhancer II were added to each flask. Cultures were returned to the incubator and grown for three days. Cultures were transferred to 50 mL Falcon tubes and supernatants were harvested by centrifugation.

### Protein Purification and Quantification

Harvested supernatants were purified via affinity chromatography on an AKTA purification system as follows. Supernatants from the replicate 40 mL cultures for each variant were filtered prior to running on FPLC using a 0.2 μm 50 mL filter unit. Filtered supernatants were applied directly to 1 mL Ni-NTA columns (GE Healthcare HisTrap HP 1 mL), washed with 20 mM Tris pH 8, 300 mM NaCl, and eluted with 20 mM Tris pH 8, 300 mM NaCl, 500 mM imidazole using a linear gradient. Several elution fractions were collected, and SDS-PAGE gel analysis was performed to determine which elution fractions to pool. Pooled elution fractions were buffer exchanged using a PD 10 desalting column (GE Healthcare) into 20 mM Tris pH 8, 300 mM NaCl, 10% glycerol. Protein preparations were quantified in triplicate by measuring absorbance at 280 nm (A280) on a Nanodrop instrument. Aliquots of 100 uL each were prepared in 1.5 mL microcentrifuge tubes, flash frozen in liquid nitrogen, and stored at −80° C. Aliquots were thawed on ice for approximately 30 minutes prior to subsequent assays.

ACE2 and Neutralizing Antibody (nAb) Binding Characterization

ACE2 and nAb binding characterization of the 17 antigens and template via Octet measurement was performed as follows. Kinetic binding assays were performed on an Octet HTX instrument at 30° C. with shaking at 1000 rpm using the 96-channel read head. Before use, human IgG Fc-capture kinetic biosensors (AHC) were pre-hydrated in 1× assay buffer (Sartorius 18-1105) for 10 min. Human Fc-tagged ACE2 standard (Sino Biological 10108-H02H) and human anti-spike nAbs (nAb1=Active Motif 91349, nAb2=Active Motif 91361, nAb3=Absolute Antibody Ab01680-10.0) were prepared at 10 nM in 1× assay buffer. Each spike ECT variant was prepared at concentrations of 12.5, 25, 50, and 100 nM in 1× assay buffer in quadruplicate from purified samples. Each tilted bottom 384-well assay plate was prepared as follows, with 50 μL/well: 1× assay buffer in Q1, 1×assay buffer in Q2, ACE2 or nAb1-3 in Q3, and spike ECT samples in Q4. The assay steps were set as follows: Baseline 1 (120 sec) in Q1 (1× assay buffer). Loading (600 sec) in Q3 (ACE2 or nAbs), Baseline 2 (120 sec) in Q2 (1× assay buffer), Association (600 sec) in Q4 (spike ECT samples), and Dissociation (600 sec) in Q2 (1× assay buffer). AHC biosensors were regenerated with 5 cycles of 10 nM glycine (pH 1.5) and neutralization in 1× assay buffer between each assay plate, with up to 4 uses per biosensor tray.

The raw data was analyzed in the Octet Data Analysis Software version 12.0. Double reference subtraction was applied and sensorgrams were aligned to the average of the baseline steps with Savitzky-Golay filtering. Sensorgrams were then globally fit to a 1:1 Langmuir binding model. Fitted data was filtered to remove traces with Full R²<0.9 and replicate measurements from 25, 50, and 100 nM spike ECT samples were averaged.

Repeat ACE2 and nAb binding characterization of the top 10 antigens and template via Octet measurement was performed as follows. Kinetic binding assays were performed on an Octet HTX instrument at 30° C. with shaking at 1000 rpm using the 8-channel read head. Before use, human IgG Fc-capture kinetic biosensors (AHC) were pre-hydrated in 1× assay buffer (Sartorius 18-1105) for 10 min. Human Fc-tagged ACE2 standard (Sino Biological 10108-H02H) and human anti-spike nAbs (nAb1=Active Motif 91349, nAb2=Active Motif 91361, nAb3=Absolute Antibody Ab01680-10.0) were prepared at 10 nM in 1× assay buffer. Each spike ECT variant was prepared at concentrations of 50 and 100 nM in 1× assay buffer in triplicate from purified samples. Each tilted bottom 384-well assay plate was prepared as follows, with 50 μL/well: 1×assay buffer in Q1, 1× assay buffer in Q2, ACE2 or nAb1-3 in Q3, and spike ECT samples in Q4. The assay steps were set as follows: Baseline 1 (60 sec) in Q1 (1× assay buffer), Loading (600 sec) in Q3 (ACE2 or nAb1-3), Baseline 2 (60 sec) in Q2 (1× assay buffer), Association (900 sec) in Q4 (spike ECT samples), and Dissociation (1800 sec) in Q2 (1× assay buffer). AHC biosensors were regenerated with 5 cycles of 10 nM glycine (pH 1.5) and neutralization in 1× assay buffer between each assay plate, with up to 4 uses per biosensor tray.

The raw data was analyzed in the Octet Data Analysis Software version 12.0. Reference well subtraction was applied and sensorgrams were aligned to the average of the baseline steps. An inter-step correction with alignment to the dissociation step at 60 sec was applied with Savitzky-Golay filtering. Sensorgrams were then globally fit to a 1:1 Langmuir binding model, with the window of interest adjusted to exclude the first 60 sec of the dissociation step. For k_{d} values flagged as “<1.0E-07 s⁻¹” in the Data Analysis Software, the k_{d} value was adjusted to 1.0E-06 s⁻¹ for further analysis (K_{D} values were calculated based on adjusted k_{d} value). Replicate measurements from 50 and 100 nM spike ECT samples were averaged.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### REFERENCES

Amanat et al. (2020) medRxiv 10.1101/2020.03.17.20037713.
Ayouba et al. (2017) J Clin Microbiol 55:165.
Berry et al. (2010) mAbs 2:53.
Buderer (1996) Acad Emerg Med 3:895.
Compoetto et al. (2017) Proc Natl Acad Sci 114:998.
Guan et al., Clin Diagn Lab Immunol 11:1148 (2004a).
Guan et al. (2004b) Clin Diagn Lab Immunol 11:287.
Healgen: (https://bit.1y/399Bi7P), accessed Match 23, 2020.
Leung et al. (2004) J Infect Dis 190:379.
Li et al. (2020) J Med Virol 10.1002/jmv.25727.
Lipsitch & Axelrod (2020) “Coronavirus may infect up to 70% of world's population. virus expert warns.” CBS News.
Liu et al. (2020) medRxiv 10.1101/2020.03.16.20035014.
Lyashchenko et al. (2000) J Immunol Methods 242:91.
Native Antigen Company: (https://bit.1y/2xV1ndU), accessed Mar. 21, 2020.
Niedrig et al. (2005) J Clin Virol 34:22.
Nishiura et al. (2020) Int J Infect Dis 10.1016/j.ijid.2020.03.020.
Qiu et al. (2005) Microbes Infect 7:882.
Raybiotech: (https://bit.1y/2QCn71e), accessed Mar. 21, 2020.
Sheridan (2020) Nature 10.1038/d41587-020-00010-2.
Walls et al. (2020) Cell. 180:1.
Wang et al. (2016) Emerg Microbes Infect 5:1.
Wrapp et al. (2020) Science 367:1260.
Wrenbeck et al. (2017) ACS Synth Biol 8:474.
Xiang et al. (2020) medRxiv 1 0.1101/2020.02.27.20028787.

What is claimed is:

1. A SARS-CoV-2 nucleocapsid protein variant, wherein the amino acid sequence of the variant comprises amino acids 254-671 of any of SEQ ID NOs: 598 to 1194 or amino acids 379-796 of any of SEQ ID NOs: 3203 to 3799.

2. A SARS-CoV-2 spike protein variant, wherein the amino acid sequence of the variant comprises amino acids 33-1230 of any of SEQ ID NOs: 1795 to 2394 or amino acids 33-705 of any of SEQ ID NOs: 4299 to 4791.

3. A SARS-CoV-2 spike protein receptor binding domain (RBD) variant, wherein the amino acid sequence of the variant comprises amino acids 33-236 of any of SEQ ID NOs: 2494 to 2592.

4. A SARS-CoV-2 nucleocapsid protein variant, wherein the amino acid sequence of the variant comprises any of SEQ ID NOs: 598 to 1194 or SEQ ID NOs: 3203 to 3799.

5. A SARS-CoV-2 spike protein variant, wherein the amino acid sequence of the variant comprises any of SEQ ID NOs: 1795 to 2394 or SEQ ID NOs: 4299 to 4791.

6. A SARS-CoV-2 spike protein receptor binding domain (RBD) variant, wherein the amino acid sequence of the variant comprises any of SEQ ID NOs: 2494 to 2592.

7. A SARS-CoV-2 nucleocapsid protein variant, wherein the amino acid sequence of the variant is amino acids 254-671 of any of SEQ ID NOs: 598 to 1194.

8. A SARS-CoV-2 nucleocapsid protein variant, wherein the amino acid sequence of the variant is amino acids 379-796 of any of SEQ ID NOs: 3203 to 3799.

9. A SARS-CoV-2 spike protein variant, wherein the amino acid sequence of the variant is amino acids 33-1230 of any of SEQ ID NOs: 1795 to 2394.

10. A SARS-CoV-2 spike protein variant, wherein the amino acid sequence of the variant is amino acids 33-705 of any of SEQ ID NOs: 4299 to 4791.

11. A SARS-CoV-2 spike protein receptor binding domain (RBD) variant, wherein the amino acid sequence of the variant is amino acids 33-236 of any of SEQ ID NOs: 2494 to 2592.

12. A SARS-CoV-2 nucleocapsid protein variant, wherein the amino acid sequence of the variant is any of SEQ ID NOs: 598 to 1194.

13. A SARS-CoV-2 nucleocapsid protein variant, wherein the amino acid sequence of the variant is any of SEQ ID NOs: 3202-3799.

14. A SARS-CoV-2 spike protein variant, wherein the amino acid sequence of the variant is any of SEQ ID NOs: 1795 to 2394.

15. A SARS-CoV-2 spike protein variant, wherein the amino acid sequence of the variant is any of SEQ ID NOs: 4299 to 4791.

16. A SARS-CoV-2 spike protein receptor binding domain (RBD) variant, wherein the amino acid sequence of the variant is any of SEQ ID NOs: 2494 to 2592.

17. A SARS-CoV-2 nucleocapsid protein variant, wherein the amino acid sequence of the variant comprises a span of at least 15 contiguous amino acids of amino acids 254-671 of any of SEQ ID NOs: 598 to 1194 or amino acids 379-796 of any of SEQ ID NOs: 3203 to 3799, and wherein the span comprises at least one variation from the corresponding span of a wild-type SARS-CoV-2 nucleocapsid protein.

18. A SARS-CoV-2 spike protein variant, wherein the amino acid sequence of the variant comprises a span of at least 15 contiguous amino acids of amino acids 33-1230 of any of SEQ ID NOs: 1795 to 2394 or amino acids 33-705 of any of SEQ ID NOs: 4299 to 4791, wherein the span comprises at least one variation from the corresponding span of a wild-type SARS-CoV-2 spike protein.

19. A SARS-CoV-2 spike protein receptor binding domain (RBD) variant, wherein the amino acid sequence of the variant comprises amino acids 33-236 of any of SEQ ID NOs: 2494 to 2592, and wherein the span comprises at least one variation from the corresponding span of a wild-type SARS-CoV-2 spike protein RBD domain.

20. A SARS-CoV-2 nucleocapsid protein variant, wherein the amino acid sequence of the variant has a substitution, insertion or deletion at one or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114,115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of the amino acid at position 1, relative to the wild-type SARS-CoV-2 nucleocapsid protein amino acid sequence (SEQ ID NO: 2594).

21. A SARS-CoV-2 nucleocapsid protein variant, wherein the amino acid sequence of the variant has a substitution at one or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of the amino acid at position 1, relative to the wild-type SARS-CoV-2 nucleocapsid protein amino acid sequence (SEQ ID NO: 2594).

22. A SARS-CoV-2 nucleocapsid protein variant, wherein the amino acid sequence of the variant has a substitution, insertion or deletion at two or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of the amino acid at position 1, relative to the wild-type SARS-CoV-2 nucleocapsid protein amino acid sequence (SEQ ID NO: 2594).

23. A SARS-CoV-2 nucleocapsid protein variant, wherein the amino acid sequence of the variant has a substitution at two or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141,144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of the amino acid at position 1, relative to the wild-type SARS-CoV-2 nucleocapsid protein amino acid sequence (SEQ ID NO: 2594).

24. A SARS-CoV-2 nucleocapsid protein variant, wherein the amino acid sequence of the variant has a substitution, insertion or deletion at three or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of the amino acid at position 1, relative to the wild-type SARS-CoV-2 nucleocapsid protein amino acid sequence (SEQ ID NO: 2594).

25. A SARS-CoV-2 nucleocapsid protein variant, wherein the amino acid sequence of the variant has a substitution at three or more of: amino acids at positions 50, 53, 54, 55, 56, 57, 59, 62, 65, 68, 72, 74, 76, 79, 81, 86, 87, 90, 91, 94, 100, 101, 105, 107, 109, 111, 113, 114, 115, 118, 120, 123, 125, 126, 127, 128, 130, 131, 134, 135, 136, 139, 140, 141, 144, 148, 150, 152, 154, 155, 157, 158, 159, 160, 161, 162, 163, 166, 167, 169, 170, 173, 188, 201, 202, 206, 250, 252, 254, 255, 257, 260, 264, 267, 268, 273, 274, 276, 280, 281, 282, 289, 290, 291, 292, 293, 294, 296, 297, 298, 299, 300, 304, 308, 311, 312, 316, 319, 321, 322, 323, 324, 325, 327, 329, 336, 338, 341, 342, 343, 347, 348, 351, 355, or 360, and a deletion of the amino acid at position 1, relative to the wild-type SARS-CoV-2 nucleocapsid protein amino acid sequence (SEQ ID NO: 2594).

26. A SARS-CoV-2 spike protein variant, wherein the amino acid sequence of the variant has a substitution, insertion or deletion at one or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion the of amino acids at positions 1 to 13, and a deletion of amino acids at positions 1212-1273, relative to the wild-type SARS-CoV-2 spike protein amino acid sequence (SEQ ID NO: 2596).

27. A SARS-CoV-2 spike protein variant, wherein the amino acid sequence of the variant has a substitution at one or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444,461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of the amino acids at positions 1 to 13, and a deletion of the amino acids at positions 1212-1273, relative to the wild-type SARS-CoV-2 spike protein amino acid sequence (SEQ ID NO: 2596).

28. A SARS-CoV-2 spike protein variant, wherein the amino acid sequence of the variant has a substitution, insertion or deletion at two or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960,961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of the amino acids at positions 1 to 13, and a deletion of the amino acids at positions 1212-1273, relative to the wild-type SARS-CoV-2 spike protein amino acid sequence (SEQ ID NO: 2596).

29. A SARS-CoV-2 spike protein variant, wherein the amino acid sequence of the variant has a substitution at two or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of the amino acids at positions 1 to 13, and a deletion of the amino acids at positions 1212-1273, relative to the wild-type SARS-CoV-2 spike protein amino acid sequence (SEQ ID NO: 2596).

30. A SARS-CoV-2 spike protein variant, wherein the amino acid sequence of the variant has a substitution, insertion or deletion at three or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444, 461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of the amino acids at positions 1 to 13, and a deletion of the amino acids at positions 1212-1273, relative to the wild-type SARS-CoV-2 spike protein amino acid sequence (SEQ ID NO: 2596).

31. A SARS-CoV-2 spike protein variant, wherein the amino acid sequence of the variant has a substitution at three or more of: amino acids at positions 32, 33, 41, 42, 46, 47, 50, 68, 71, 77, 78, 83, 84, 87, 90, 94, 95, 100, 101, 108, 112, 113, 114, 129, 132, 134, 135, 137, 138, 140, 141, 142, 153, 155, 157, 159, 171, 172, 173, 174, 175, 176, 177, 179, 180, 181, 187, 188, 202, 206, 207, 208, 222, 224, 226, 227, 228, 237, 241, 243, 245, 249, 259, 261, 262, 266, 271, 273, 276, 279, 280, 284, 292, 293, 297, 299, 302, 324, 325, 335, 339, 345, 352, 354, 356, 378, 399, 405, 417, 444,461, 472, 477, 514, 529, 530, 537, 540, 541, 546, 547, 554, 556, 569, 570, 572, 573, 575, 580, 582, 586, 588, 598, 606, 607, 619, 622, 623, 626, 627, 630, 646, 655, 668, 682, 683, 684, 685, 688, 690, 693, 704, 706, 711, 713, 719, 723, 726, 727, 732, 734, 739, 747, 748, 750, 754, 761, 764, 768, 772, 774, 778, 779, 780, 788, 817, 826, 831, 834, 838, 839, 841, 844, 845, 855, 858, 859, 872, 875, 876, 878, 879, 882, 884, 895, 912, 921, 922, 929, 932, 933, 936, 937, 939, 940, 942, 943, 946, 953, 955, 960, 961, 977, 981, 982, 986, 987, 990, 991, 993, 1016, 1023, 1027, 1034, 1038, 1040, 1041, 1045, 1055, 1060, 1070, 1072, 1080, 1084, 1088, 1101, 1103, 1104, 1113, 1114, 1125, 1130, or 1133, and a deletion of the amino acids at positions 1 to 13, and a deletion of the amino acids at positions 1212-1273, relative to the wild-type SARS-CoV-2 spike protein amino acid sequence (SEQ ID NO: 2596).

32. A SARS-CoV-2 spike protein variant, wherein the amino acid sequence of the variant has a substitution, insertion or deletion at one or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

33. A SARS-CoV-2 spike protein variant, wherein the amino acid sequence of the variant has a substitution at one or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

34. A SARS-CoV-2 spike protein variant, wherein the amino acid sequence of the variant has a substitution, insertion or deletion at two or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264,284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 2596).

35. A SARS-CoV-2 spike protein variant, wherein the amino acid sequence of the variant has a substitution at two or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419,420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 25%).

36. A SARS-CoV-2 spike protein variant, wherein the amino acid sequence of the variant has a substitution, insertion or deletion at three or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399, 417, 419, 420, 424, 432, 439, 443, 445, 459, 460, 461, 464, 467, 470, 472, 475, 477, 480, 482, 484, 487, 488, 489, 490, 496, 501, 503, 504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 25%).

37. A SARS-CoV-2 spike protein variant, wherein the amino acid sequence of the variant has a substitution at three or more of: amino acids at positions 36, 46, 47, 49, 50, 63, 64, 68, 70, 71, 81, 84, 92, 94, 95, 97, 98, 101, 108, 112, 115, 123, 125, 129, 132, 136, 138, 140, 146, 153, 155, 159, 161, 167, 171, 172, 177, 178, 184, 186, 187, 188, 192, 202, 205, 206, 207, 209, 213, 221, 222, 225, 226, 228, 236, 237, 239, 240, 241, 245, 249, 252, 257, 258, 260, 262, 264, 284, 292, 298, 299, 302, 316, 321, 324, 325, 329, 335, 338, 339, 344, 345, 350, 354, 356, 367, 368, 375, 377, 378, 379, 393, 399,417, 419, 420, 424, 432, 439, 443, 445, 459, 460, 461, 464, 467, 470, 472, 475, 477, 480, 482, 484, 487, 488, 489, 490, 496, 501, 503,504, 505, 514, 517, 518, 519, 522, 526, 528, 530, 534, 537, 546, 551, 557, 566, 567, 568, 569, 571, 573, 574, 582, 585, 588, 592, 595, 596, 601, 603, 608, 621, 622, 623, 624, 625, 627, 630, 635, 636, 637, 642, 651, 653, 655, 666, 668, 673, 678, 682, 683, and/or 685, and a deletion of amino acids positions at 1 to 13, and a deletion of amino acids at positions 687-1273, relative to the wild-type spike protein amino acid sequence (SEQ ID NO: 25%).

38. A SARS-CoV-2 spike protein variant, wherein the amino acid sequence of the variant has a substitution, insertion or deletion at one or more of: amino acids at positions 115, 200, 370, 372, 375, 376 and/or 415 and a deletion of amino acids positions at 1 to 13, and a deletion of amino acids at positions 687-1273, relative to the wild-type spike protein amino acid sequence (SEQ ID NO: 2596).

39. A SARS-CoV-2 spike protein variant, wherein the amino acid sequence of the variant has a substitution at one or more of amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

40. A SARS-CoV-2 spike protein variant, wherein the amino acid sequence of the variant has a substitution, insertion or deletion at two or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

41. A SARS-CoV-2 spike protein variant, wherein the amino acid sequence of the variant has a substitution at two or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

42. A SARS-CoV-2 spike protein variant, wherein the amino acid sequence of the variant has a substitution, insertion or deletion at three or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

43. A SARS-CoV-2 spike protein variant, wherein the amino acid sequence of the variant has a substitution at three or more of: amino acids at positions 115, 200, 370, 372, 375, 376 and/or 415, and a deletion of amino acids positions at 1 to 13, and a deletion of amino acids at positions 687-1273, relative to the wild-type spike protein amino acid sequence (SEQ ID NO: 2596).

44. A SARS-CoV-2 spike protein variant, wherein the amino acid sequence of the variant has a substitution, insertion or deletion at one or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466,467, 469, 470, 471, 472, 477, 478, 479, 480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

45. A SARS-CoV-2 spike protein variant, wherein the amino acid sequence of the variant has a substitution at one or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

46. A SARS-CoV-2 spike protein variant, wherein the amino acid sequence of the variant has a substitution, insertion or deletion at two or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466,467, 469, 470, 471, 472, 477, 478, 479, 480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

47. A SARS-CoV-2 spike protein variant, wherein the amino acid sequence of the variant has a substitution at two or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399,401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488, 491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

48. A SARS-CoV-2 spike protein variant, wherein the amino acid sequence of the variant has a substitution, insertion or deletion at three or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488, 491, 496, 512, 514, 518, 519, 520, 522, 528, 546, 558, 569, 588, 591, 697, 701, 702, 704,705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

49. A SARS-CoV-2 spike protein variant, wherein the amino acid sequence of the variant has a substitution at three or more of: amino acids at positions 41, 115, 226, 282, 319, 330, 331, 332, 338, 341, 348, 349, 351, 352, 353, 357, 358, 359, 360, 362, 363, 364, 365, 366, 367, 369, 370, 371, 372, 373, 375, 376, 385, 388, 389, 390, 393, 399, 401, 407, 408, 409, 410, 417, 418, 419, 422, 424, 438, 439, 440, 443, 445, 461, 462, 464, 465, 466, 467, 469, 470, 471, 472, 477, 478, 479, 480, 481, 482, 488,491, 496, 512, 514, 518, 519, 520, 522, 528, 546, 558, 569, 588, 591, 697, 701, 702, 704, 705, 711, 787, 869, 890, 895, 899, 904, 963, 964, 968, 969, 978, 979, 984, 985, 988, 1030, 1040, 1041, 1045, 1047, 1072, 1144, and/or 1145 and a deletion of amino acids positions at 1 to 13, and a deletion of amino acids at positions 1212 to 1273, relative to the wild-type spike protein amino acid sequence (SEQ ID NO: 2596).

50. A SARS-CoV-2 spike protein receptor binding domain (RBD) variant, wherein the amino acid sequence of the variant has a substitution, insertion or deletion at one or more of: amino acids at positions 335, 339, 340, 356, 357, 362, 366, 368, 372, 393, 394, 399, 402, 403, 404,405, 406, 410, 414, 417, 438, 439, 450, 477, 478, 483, 484, 486, 489, 490, 501, 502, or 504, and a deletion of the amino acids at positions 1 to 327 and a deletion of the amino acids at positions 532 to 1273, relative to the wild-type SARS-CoV-2 spike protein amino acid sequence (SEQ ID NO: 2596).

51. A SARS-CoV-2 spike protein receptor binding domain (RBD) variant, wherein the amino acid sequence of the variant has a substitution at one or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of the amino acids at positions 1 to 327 and a deletion of the amino acids at positions 532 to 1273, relative to the wild-type SARS-CoV-2 spike protein amino acid sequence (SEQ ID NO: 2596).

52. A SARS-CoV-2 spike protein receptor binding domain (RBD) variant, wherein the amino acid sequence of the variant has a substitution, insertion or deletion at two or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of the amino acid at positions 1 to 327 and a deletion of the amino acids at positions 532 to 1273, relative to the wild-type SARS-CoV-2 spike protein amino acid sequence (SEQ ID NO: 2596).

53. A SARS-CoV-2 spike protein receptor binding domain (RBD) variant, wherein the amino acid sequence of the variant has a substitution at two or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of the amino acids at positions 1 to 327 and a deletion of the amino acids at positions 532 to 1273, relative to the wild-type SARS-CoV-2 spike protein amino acid sequence (SEQ ID NO: 2596).

54. A SARS-CoV-2 spike protein receptor binding domain (RBD) variant, wherein the amino acid sequence of the variant has a substitution, insertion or deletion at three or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of the amino acids at positions 1 to 327 and a deletion of the amino acids at positions 532 to 1273, relative to the wild-type SARS-CoV-2 spike protein amino acid sequence (SEQ ID NO: 2596).

55. A SARS-CoV-2 spike protein receptor binding domain (RBD) variant, wherein the amino acid sequence of the variant has a substitution at three or more of: amino acids at positions 335, 339, 340, 356, 357, 362, 366, 368, 372, 393, 394, 399, 402, 403, 404, 405, 406, 410, 414, 417, 438, 439, 450, 477, 478, 483, 484, 486, 489, 490, 501, 502, or 504, and a deletion of the amino acids at positions 1 to 327 and a deletion of the amino acids at positions 532 to 1273, relative to the wild-type SARS-CoV-2 spike protein amino acid sequence (SEQ ID NO: 2596).

56. A SARS-CoV-2 spike protein receptor binding domain (RBD) variant, wherein the amino acid sequence of the variant has a substitution, insertion or deletion at one or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469,470, 471, 472, 479, 480, 481, 482, 488, 491,496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

57. A SARS-CoV-2 spike protein receptor binding domain (RBD) variant, wherein the amino acid sequence of the variant has a substitution at one or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

58. A SARS-CoV-2 spike protein receptor binding domain (RBD) variant, wherein the amino acid sequence of the variant has a substitution, insertion or deletion at two or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

59. A SARS-CoV-2 spike protein receptor binding domain (RBD) variant, wherein the amino acid sequence of the variant has a substitution at two or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406,407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

60. A SARS-CoV-2 spike protein receptor binding domain (RBD) variant, wherein the amino acid sequence of the variant has a substitution, insertion or deletion at three or more of: amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

61. A SARS-CoV-2 spike protein receptor binding domain (RBD) variant, wherein the amino acid sequence of the variant has a substitution at three or more of amino acids at positions 330, 331, 332, 338, 341, 348, 349, 351, 352, 353, 358, 362, 363, 364, 365, 366, 367, 370, 371, 373, 376, 384, 391, 392, 393, 394, 395, 399, 401, 406, 407, 408, 409, 418, 419, 422, 424, 434, 439, 440, 443, 445, 453, 456, 461, 462, 464, 465, 466, 467, 469, 470, 471, 472, 479, 480, 481, 482, 488, 491, 496, 504, 508, 512, 514, 518, 519 and/or 520 and a deletion of amino acids at positions 1 to 327 and a deletion of amino acids at positions 532-1273, relative to the wild-type spike protein amino acid sequence (SEQ ID NO: 2596).

62. A SARS-CoV-2 nucleocapsid protein variant comprising amino acids 254-671 of an amino acid sequence set forth in SEQ ID NOs: 598 to 1194 or 3203 to 3799.

63. A SARS-CoV-2 spike protein variant comprising amino acids 33-1230 of an amino acid sequence set forth in SEQ ID NOs: 1795 to 2394 or 4299 to 4791.

64. A SARS-CoV-2 spike protein receptor binding domain (RBD) variant comprising amino acids 33-236 of an amino acid sequence set forth in 2494 to 2592.

65. A composition comprising the variant of any of the preceding claims.

66. The composition of claim 65, wherein the variant is immobilized on a solid support.

67. The composition of claim 65, wherein the composition is a vaccine composition.

68. The composition of claim 67, further comprising a pharmaceutically acceptable carrier.

69. The composition of claim 67 or 68, further comprising an adjuvant.

70. A nucleic acid encoding the variant of any one of claims 1 to 64.

71. The nucleic acid of claim 70, wherein the nucleic acid has a sequence comprising any of SEQ ID NOs: 1 to 597 or SEQ ID NOs. 2606 to 3202, or the complementary sequence thereof.

72. The nucleic acid of claim 70, wherein the nucleic acid has a sequence comprising any of SEQ ID NOs: 1195 to 1794 or SEQ ID NOs. 3806 to 4298, or the complementary sequence thereof.

73. The nucleic acid of claim 70, wherein the nucleic acid has a sequence comprising any of SEQ ID NOs: 2395 to 2493, or the complementary sequence thereof.

74. The nucleic acid of any one of claims 70 to 73, wherein the nucleic acid comprises DNA.

75. The nucleic acid of any one of claims 70 to 73, wherein the nucleic acid comprises RNA.

76. The nucleic acid of claim 75, wherein the nucleic acid is an mRNA molecule.

77. A composition comprising the nucleic acid of any one of claims 70 to 73.

78. The composition of claim 77, wherein the composition is a vaccine composition.

79. The composition of claim 78, further comprising a pharmaceutically acceptable carrier.

80. The composition of claim 78 or 79, further comprising an adjuvant.

81. A vector comprising the nucleic acid of any one of claims 70 to 73.

82. The vector of claim 81, wherein the vector is an expression vector.

83. A composition comprising the vector of claim 81 or claim 82.

84. The composition of claim 83, wherein the composition is a vaccine composition.

85. The composition of claim 83, further comprising a pharmaceutically acceptable carrier.

86. The composition of claim 83 or 84, further comprising an adjuvant.

87. A cell comprising the nucleic acid of any one of claims 70 to 76.

88. The cell of claim 87, wherein the cell expresses the variant encoded by the nucleic acid.

89. The cell of claim 87 or 88, wherein the cell is a prokaryotic cell.

90. The cell of claim 87, wherein the cell is a bacterial cell.

91. The cell of claim 90, wherein the cell is an E. coli cell.

92. The cell of claim 87 or 88, wherein the cell is a eukaryotic cell.

93. The cell of claim 92, wherein the cell is a yeast cell.

94. The cell of claim 93, wherein the cell is a Saccharomyces cerevisiae cell.

95. The cell of claim 87, wherein the cell is a mammalian cell.

96. The cell of claim 95, wherein the cell is a human cell.

97. A diagnostic test kit comprising a variant of any one of claims 1 to 64, wherein the variant is a SARS-CoV-2 nucleocapsid protein variant, and wherein the kit further comprises a reagent capable of detecting an immunological complex which comprises an antibody bound to the variant.

98. A diagnostic test kit comprising a variant of any one of claims 1 to 64, wherein the variant is a SARS-CoV-2 spike protein variant, and wherein the kit further comprises a reagent capable of detecting an immunological complex which comprises an antibody bound to the variant.

99. A diagnostic test kit comprising a variant of any one of claims 1 to 64, wherein the variant is a SARS-CoV-2 spike protein receptor binding domain (RBD) variant, and wherein the kit further comprises a reagent capable of detecting an immunological complex which comprises an antibody bound to the variant.

100. A diagnostic test kit comprising at least two of the variants of any one of claim 1 to 64, wherein the two variants are selected from a SARS-CoV-2 nucleocapsid protein variant; a SARS-CoV-2 spike protein variant; and a SARS-CoV-2 spike protein receptor binding domain (RBD) variant.

101. The diagnostic test kit of claim 100 wherein the kit further comprises a reagent capable of detecting an immunological complex which comprises an antibody bound to the variant.

102. The diagnostic test kit of any one of claims 97 to 101, wherein the kit comprises one or more control standards and/or a specimen diluent and/or washing buffer.

103. The diagnostic test kit of any one of claims 97 to 102, wherein the variant is immobilized on a solid support.

104. The diagnostic test kit of any one of claims 97 to 103, wherein the variant is immobilized on a solid support, wherein the solid support is a microplate well.

105. The diagnostic test kit of any one of claims 97 to 104, wherein the immunological complex is detected by ELISA.

106. The diagnostic test kit of any one of claims 97 to 104, wherein the immunological complex is detected by lateral flow.

107. A method of determining whether a subject is infected with SARS-CoV-2, the method comprising:
a) contacting a sample from the subject with any one of the protein variants of any one of claims 1 to 64, and
b) detecting binding of the protein variant with a SARS-CoV-2 antibody present in the sample.

108. The method of claim 107, wherein the biological sample is a serum sample.

109. The method of claim 107 or 108, wherein the protein variant is immobilized on a solid support.

110. The method of claim 109, wherein the solid support is a microplate well and/or a flow cell.

111. The method of any of claims 107 to 110, wherein the immunological complex is detected by ELISA.

112. The method of any of claims 107 to 110, wherein the immunological complex is detected by lateral flow.

113. A method of treating and/or preventing a SARS-CoV-2 infection in a subject comprising administering to the subject a composition according to any one of claims 83 to 86.

114. The method of claim 113, wherein the composition is administered intravenously.

115. The method of claim 113 or 114, comprising administering one dose of the composition.

116. The method of claim 113 or 114, comprising administering multiple doses of the composition.
